SEQUENCE LISTING (114 sequences)

SEQ ID NO: 1 - DNA, 993 bp, Escherichia coli
  source 1..993: mol_type = genomic DNA
atgagtcagaatacgctgaaagttcatgatttaaatgaagatgcggaatttgatgagaacggagttgaggtttttgacgaaaaggccttagtagaactggaacccagtgataacgatttggccgaagaggaactgttatcgcagggagccacacagcgtgtgttggacgcgactcagctttaccttggtgagattggttattcaccactgttaacggccgaagaagaagtttattttgcgcgtcgcgcactgcgtggagatgtcgcctctcgccgccggatgatcgagagtaacttgcgtctggtggtaaaaattgcccgccgttatggcaatcgtggtctggcgttgctggaccttatcgaagagggcaacctggggctgatccgcgcggtagagaagtttgacccggaacgtggtttccgcttctcaacatacgcaacctggtggattcgccagacgattgaacgggcgattatgaaccaaacccgtactattcgtttgccgattcacatcgtaaaggagctgaacgtttacctgcgaaccgcacgtgagttgtcccataagctggaccatgaaccaagtgcggaagagatcgcagagcaactggataagccagttgatgacgtcagccgtatgcttcgtcttaacgagcgcattacctcggtagacaccccgctgggtggtgattccgaaaaagcgttgctggacatcctggccgatgaaaaagagaacggtccggaagataccacgcaagatgacgatatgaagcagagcatcgtcaaatggctgttcgagctgaacgccaaacagcgtgaagtgctggcacgtcgattcggtttgctggggtacgaagcggcaacactggaagatgtaggtcgtgaaattggcctcacccgtgaacgtgttcgccagattcaggttgaaggcctgcgccgtttgcgcgaaatcctgcaaacgcaggggctgaatatcgaagcgctgttccgcgagtaa

SEQ ID NO: 2 - DNA, 1842 bp, Escherichia coli
  source 1..1842: mol_type = genomic DNA
ttaatcgtccaggaagctacgcagcacttcagaacggctcgggtgacgcagtttgcgcagcgccttcgcttcgatctgacggatacgttcgcgggtaacgtcgaactgtttacccacttcttccagcgtgtagtcggtgttcatatcgataccgaaacgcatacgcagaacttttgcttcacgcgcggtcaggccagccagcacgtcgtgcgttgccgcacgcaggctttcggtggtcgcagaatccagcggcagctcgagggtggtatcctcgatgaaatcccccagatgcgaatcttcatcatcaccgatcggcgtttccatggagattggctctttggcgatcttcagcactttgcggatcttgtcttccggcatcagcatacgttcagccagttcttccggcgtcggttcacggcccatctcttgcagcatctggcgagaaatacggttgagcttgttgatggtctcaatcatatgcaccggaatacggatggtgcgcgcctgatccgcgatagagcgggtgatcgcctgacggatccaccaggttgcgtaggtggagaacttgtaaccacggcggtattcgaatttatcaaccgctttcatcagaccgatgttgccttcctgaatcaggtcaaggaactgcaagccacggttggtgtatttcttagcgatagaaataaccagacgtaagttcgcttcaaccatctctttcttcgcacggcgggctttcgcttcaccgatggacatacgacggttgatatctttaacctgctcgatggtcaggccggtttcttcttcaatctgctgcagtttttgcagggcgcgatgcacttcttcagagacatcgtgcagtttttccgaccacggcttgttcatcgcaattgccgcgttgaaccaggtatcgctggtttcgttgccggtaaacagggtaatgaagtttttcttcggcattttgcactgctcaacgcagagcttcatgatcagacgttcttgcgtacgaacgcggtccatcatgacgcgcatgctgttgaccaggtagtcaaactgcttcggcaccaggcggaactgtttgaatacttcagacagtttcaggatctcttcctgagcggtagcgtgactgcgacctttcgctttgatggtgtcacgcgttacaacgtactgagcgcgtagttccgcaaatttttcgcgagccagttccgggtcgatgctgttgtcatcatcggcgctgtcgtcatcgccatcttcttcgtcttcatcttcgtcatcgtccagatcttcctgggaaagctcagaaccgacgtgagtggcggtaggtgccagatcttcttctgcgttcgggtcaacaaagccggtgatcagatcggacagacgcgcttcttctgcttcaacacgatcgtactgttccagcagataggtgatcgcttccggatattcagcaacggagcattgaacctggttgatcccgtcttcaatacgcttagcgatgtcaatttcgccttcgcgggtcaacagttcaacggtgcccatttcacgcatgtacatgcgtaccgggtcagtcgtgcgcccgatttcagattccacgctggaaagcacctgcgcggcggcttcggcagcatcttcgtccgcggtgttttcagccagcatcagatcatcggcatccggtgcttcttccatcacctgaatgcccatgtcgttgatcatttggatgatgtcttcgatctgatctgaatcgacgatatcttccggcagatggtcattgacctcggcataggtcagatagccttgctccttaccacgggtgacaagaagtttcagctgtgactgcgggttttgctccat

SEQ ID NO: 3 - DNA, 855 bp, Escherichia coli
  source 1..855: mol_type = genomic DNA
atgactgacaaaatgcaaagtttagctttagccccagttggcaacctggattcctacatccgggcagctaacgcgtggccgatgttgtcggctgacgaggagcgggcgctggctgaaaagctgcattaccatggcgatctggaagcagctaaaacgctgatcctgtctcacctgcggtttgttgttcatattgctcgtaattatgcgggctatggcctgccacaggcggatttgattcaggaaggtaacatcggcctgatgaaagcagtgcgccgtttcaacccggaagtgggtgtgcgcctggtctccttcgccgttcactggatcaaagcagagatccacgaatacgttctgcgtaactggcgtatcgtcaaagttgcgaccaccaaagcgcagcgcaaactgttcttcaacctgcgtaaaaccaagcagcgtctgggctggtttaaccaggatgaagtcgaaatggtggcccgtgaactgggcgtaaccagcaaagacgtacgtgagatggaatcacgtatggcggcacaggacatgacctttgacctgtcttccgacgacgattccgacagccagccgatggctccggtgctctatctgcaggataaatcatctaactttgccgacggcattgaagatgataactgggaagagcaggcggcaaaccgtctgaccgacgcgatgcagggtctggacgaacgcagccaggacatcatccgtgcgcgctggctggacgaagacaacaagtccacgttgcaggaactggctgaccgttacggcgtttccgctgagcgtgtacgccagctggaaaagaacgcgatgaaaaaattgcgtgctgccattgaagcgtaa

SEQ ID NO: 4 - DNA, 576 bp, Escherichia coli
  source 1..576: mol_type = genomic DNA
atgagcgagcagttaacggaccaggtcctggttgaacgggtccagaagggagatcagaaagcctttaacttactggtagtgcgctatcagcataaagtggcgagtctggtttcccgctatgtgccgtcgggtgatgttcccgatgtggtacaagaagcttttattaaagcctatcgtgcgctggattcgttccggggagatagcgctttttatacatggctgtatcggattgctgtaaatacagcgaaaaattacctggttgctcaggggcgtcgtccaccttccagtgatgtggatgccattgaagctgaaaacttcgaaagtggcggcgcgttgaaagaaatttcgaaccctgagaacttaatgttgtcagaagaactgagacagatagttttccgaactattgagtccctcccggaagatttacgcatggcaataaccttgcgggagctggatggcctgagctatgaagagatagccgctatcatggattgtccggtaggtacggtgcgttcacgtatcttccgagcgagggaagctattgataacaaagttcaaccgcttatcaggcgttga

SEQ ID NO: 5 - DNA, 1434 bp, Escherichia coli
  source 1..1434: mol_type = genomic DNA
tcaaacgagttgtttacgctggtttgacggcggaatggataaagactctcggtacttcgcaacagtgcggcgtgccaccatgataccttgttccgacagcaaagaggttaacttgctgtcgctcaacggtttcgctgggttttccgccgcgattaatttcttcaccagcgcacgaatcgccgtggaggaagcttcgccgccgccctcggtattgacgtgactggaaaagaaatacttcagttcaaaaatgcctcgtggactatgcaggtatttttgcgtggtcacgcgagatatcgtcgattcatgcatttcgacagcctgggcgatatcggccagtaccatcggtttcatatattcttcaccttgctcaaagaaggcttgctgctgttcaacgatacagcgactcacgcgcagtagcgtatcgttacggctttccagactcttgatcaaccatttggcatcctgcagattgctgcggataaactggctgtcaccatcgttgcgcgcgttattgcacatcgaggcgtagtgctggttgatttgcagacgcggaatgctgtcactgttgagttctaccgtccagtgaccgttatgcttacgcaccagcacatctggaatgacatactcaggttcgccagtctggatcgactgcccggggcgcggatcgagcgactggatcagattgacggcttctttcagcacatcttctttcagacgcgtgacgcgcattaaagtgcggaagtcgtgattggctaacagatcgagatgatcgctaatgatcagtctggcctcttccagccacggcgtggtcttatcgaattgggagagttggatcagcagacagtcacgcagatcttttgccgccacaccgaccggatcaaaccgttggatccgcttaaggacggcttcaacttcgtcgatgtcgatctcttcatcgcctatactttcgagaatatcttccagcgggacagtcagataaccggtttcatcaacggcatcgacgatagaggtagcaatagcgcggtcagtgtcggaaaacggtgtcagctcaacctgccacatcaggtaatcctgcaaggtctgcgtcgtttcgccctggtagaccggcagctcgtcgtcaatgtagtcaccgctggtgccggatggtgtaccagcggtgtaaatggtgtcccaactggcatcgagcggcagctcttccggcatctctttttgttcgagcgcgtcggcggtgtccagcgtttcactgtcttgcgtttcgcgggtgtcgatttcttcatgagtgtcgatttgctcaagcagcggattactctccagcgcctgctgtagctcctgctgaagttccagcgtcgacaactgcaacagacgaattgcctgttggagctgtggcgtcatcgccagttgttggctaagcctgagttgcaaaccttgcttcat

SEQ ID NO: 6 - DNA, 720 bp, Escherichia coli
  source 1..720: mol_type = genomic DNA
gtgaattcactctataccgctgaaggtgtaatggataaacactcgctgtggcagcgttatgtcccgctggtgcgtcacgaagcattgcgcctgcaggttcgactgcccgcgagcgtggaacttgacgatctgctacaggcgggcggcattgggttacttaatgccgtcgaacgctatgacgccctacaaggaacggcatttacaacttacgcagtgcagcgtatccgtggcgctatgctggatgaacttcgcagccgtgactgggtgccgcgcagcgtgcgacgcaacgcgcgtgaagtggcacaggcaatagggcaactggagcaggaacttggccgcaacgccacggaaactgaggtagcggaacgtttagggatcgatattgccgattatcgccaaatgttgctcgacaccaataacagccagctcttctcctacgatgagtggcgcgaagagcacggcgatagcatcgaactggttactgatgatcatcagcgagaaaacccgctacaacaactactggacagtaatctgcgccagcgggtgatggaagccatcgaaacgttgccggagcgcgaaaaactggtattaaccctctattaccaggaagagctgaatctcaaagagattggcgcggtgctggaggtcggggaatcgcgggtcagtcagttacacagccaggctattaaacggttacgcactaaactgggtaagttataa

SEQ ID NO: 7 - DNA, 276 bp, Escherichia coli
  source 1..276: mol_type = genomic DNA
ttaacgacgaccttcagcaatagcggtaacggcttgtaattcagcggcttcctgctcttgctgttcctggcgttcgcgaacgtcgaggatctggttgttgatcagaccttcttcgatttcgcgcagcgcgattacagtggttttatcgttttcttccggtaccagcggatcctttccgcctacctgcatctgacgagcgcgacgcgcggcgaccagtaccaggtcaaaacggttaccaattttctctacagcgtcctgaacagttacgcgtgccat

SEQ ID NO: 8 - DNA, 402 bp, Escherichia coli
  source 1..402: mol_type = genomic DNA
tcacgccgttaacttcaccggctcgtcacgaaaatcatccgccggttccagcttcagattcagcgtcgtcagcagttcacgcagcttctcgtgaaactcacgcagggtgtgctccagtcgttcaaccacttcagtgtcttttaccggaacactcttccagtcgcctgctttatcgaacagaccaaactggtaactgtaggtaaaacgggattcctgcgcttccagctccatccaccagccccagaattcacgcacttccggtgccggtttcacgttgacgcatacagccagacaatcgaaaaagaatcgattatctttgcacttaccttcacgaatatacgggcctagtgcggtaaattttttgatcaatctgctcttcgggtgtccactcggtaacgtcat

SEQ ID NO: 9 - DNA, 121 bp, Escherichia coli
  source 1..121: mol_type = genomic DNA
aaaaaatgaccccggctagaccggggtgcgcgaatactgcgccaacaccagggaaatcttggctgcgccgtaaattattatgatgagttacaagggcacagcactgtttttcaggccgcac

SEQ ID NO: 10 - DNA, 456 bp, Escherichia coli
  source 1..456: mol_type = genomic DNA
atgcaagaagggcaaaaccgtaaaacatcgtccctgagtattctcgccatcgctggggtggaaccatatcaggagaagccgggcgaagagtatatgaatgaagcccagctggcgcacttccgtcgtattctggaagcatggcgtaatcaactcagggatgaagtcgatcgcaccgttacacatatgcaggatgaagcagccaacttcccggacccggtagaccgtgcagcccaggaagaagagttcagcctcgaactgcgtaaccgcgatcgcgagcgtaagctgatcaaaaagatcgagaagacgctgaaaaaagtggaagacgaagatttcggctactgcgaatcctgcggtgttgaaattggtattcgccgtctggaagcgcgcccgacagccgatctgtgcatcgactgcaaaacgctggctgaaattcgcgaaaaacagatggctggctaa

SEQ ID NO: 11 - DNA, 825 bp, Escherichia coli
  source 1..825: mol_type = genomic DNA
atgcaatcactacatgggaattgtctaattgcgtatgcaagacataaatatattctcaccatggttaatggtgaatatcgctattttaatggcggtgacctggtttttgcggatgcaagccaaattcgagtagataagtgtgttgaaaattttgtattcgtgtcaagggacacgctttcattatttctcccgatgctcaaggaggaggcattaaatcttcatgcacataaaaaagtttcttcattactcgttcatcactgtagtagagatattcctgtttttcaggaagttgcgcaactatcgcagaataagaatcttcgctatgcagaaatgctacgtaaaagagcattaatctttgcgttgttatctgtttttcttgaggatgagcactttataccgctgcttctgaacgttttacaaccgaacatgcgaacacgagtttgtacggttatcaataataatatcgcccatgagtggacactagcccgaatcgccagcgagctgttgatgagtccaagtctgttaaagaaaaaattgcgcgaagaagagacatcatattcacagttgcttactgagtgtagaatgcaacgtgctttgcaacttattgttatacatggtttttcaattaagcgagttgcagtatcctgtggatatcacagcgtgtcgtatttcatttacgtctttcgaaattattatgggatgacgcccacagagtatcaggagcgatcggcgcagagattgtcgaaccgtgactcggcggcaagtattgttgcgcaagggaatttttacggcactgaccgttctgcggaaggaataagattatag

SEQ ID NO: 12 - DNA, 91 bp, Escherichia coli
  source 1..91: mol_type = genomic DNA
aacacatcagatttcctggtgtaacgaattttttaagtgcttcttgcttaagcaagtttcatcccgaccccctcagggtcgggattttttt

SEQ ID NO: 13 - DNA, 1890 bp, Escherichia coli
  source 1..1890: mol_type = genomic DNA
atggctgaattcgaaaccacttttgcagatctgggcctgaaggctcctatccttgaagcccttaacgatctgggttacgaaaaaccatctccaattcaggcagagtgtattccacatctgctgaatggccgcgacgttctgggtatggcccagacggggagcggaaaaactgcagcattctctttacctctgttgcagaatcttgatcctgagctgaaagcaccacagattctggtgctggcaccgacccgcgaactggcggtacaggttgctgaagcaatgacggatttctctaaacacatgcgcggcgtaaatgtggttgctctgtacggcggccagcgttatgacgtgcaattacgcgccctgcgtcaggggccgcagatcgttgtcggtactccgggccgtctgctggaccacctgaaacgtggcactctggacctctctaaactgagcggtctggttctggatgaagctgacgaaatgctgcgcatgggcttcatcgaagacgttgaaaccattatggcgcagatcccggaaggtcatcagaccgctctgttctctgcaaccatgccggaagcgattcgtcgcattacccgccgctttatgaaagagccgcaggaagtgcgcattcagtccagcgtgactacccgtcctgacatcagccagagctactggactgtctggggtatgcgcaaaaacgaagcactggtacgtttcctggaagcggaagattttgatgcggcgattatcttcgttcgtaccaaaaacgcgactctggaagtggctgaagctcttgagcgtaacggctacaacagcgccgcgctgaacggtgacatgaaccaggcgctgcgtgaacagacactggaacgcctgaaagatggtcgtctggacatcctgattgcgaccgacgttgcagcccgtggcctggacgttgagcgtatcagcctggtagttaactacgatatcccgatggattctgagtcttacgttcaccgtatcggtcgtaccggtcgtgcgggtcgtgctggccgcgcgctgctgttcgttgagaaccgcgagcgtcgtctgctgcgcaacattgaacgtactatgaagctgactattccggaagtagaactgccgaacgcagaactgctaggcaaacgccgtctggaaaaattcgccgctaaagtacagcagcagctggaaagcagcgatctggatcaataccgcgcactgctgagcaaaattcagccgactgctgaaggtgaagagctggatctcgaaactctggctgcggcactgctgaaaatggcacagggtgaacgtactctgatcgtaccgccagatgcgccgatgcgtccgaaacgtgaattccgtgaccgtgatgaccgtggtccgcgcgatcgtaacgaccgtggcccgcgtggtgaccgtgaagatcgtccgcgtcgtgaacgtcgtgatgttggcgatatgcagctgtaccgcattgaagtgggccgcgatgatggtgttgaagttcgtcatatcgttggtgcgattgctaacgaaggcgacatcagcagccgttacattggtaacatcaagctgtttgcttctcactccaccatcgaactgccgaaaggtatgccgggtgaagtgctgcaacactttacgcgcactcgcattctcaacaagccgatgaacatgcagttactgggcgatgcacagccgcatactggcggtgagcgtcgtggcggtggtcgtggtttcggtggcgaacgtcgtgaaggcggtcgtaacttcagcggtgaacgccgtgaaggtggccgtggtgatggtcgtcgttttagcggcgaacgtcgtgaaggccgcgctccgcgtcgtgatgattctaccggtcgtcgtcgtttcggtggtgatgcgtaa

SEQ ID NO: 14 - DNA, 106 bp, Escherichia coli
  source 1..106: mol_type = genomic DNA
aaaaaaaagcccatcgtgggagatgggcaaagactacacacagcaattcgttgtttcactcaggggatttccatgcttataaatcaatatgttgatttataaccgt

SEQ ID NO: 15 - DNA, 1014 bp, Escherichia coli
  source 1..1014: mol_type = genomic DNA
tcattctgcagacaacatcaagcgcagtcgaccaccggttccccatatttggcattgccaggcatcgcatcgctggctcaattgattcaaataagcgttacctaaagtgcctaacggaacaccattactgatttgcacctggtgttcgccggtatttaacgtcgcattcagacccgcagaaaccagaatgagatttttcagttcgcgatgataatagccaactaatagcggaaactgccccggcagattggcctgacgaagtaaatggtttacctgcttcaataacgcgcccaactctggcaaccgttgattttggtgtgcaagctgttcctgtaataatccgttaaacaatgcgcgtaataacaaggcagcaagtacgccattatgtccagctcgggtgacatcaaggcaataaaatgccagatcgttttccgaaagtgcggcaatatcaagcaccaggccgggtttgtccgcggcaaccaattgacgataattaacccggcaatgggaaatcacctgctgaaccggcggttgtagttcctgtaataatttcgccgctgcggcagggttatcaaccattgcatcctagtcgcgaaaaagcctttcctcttcctcaacgcgcgaattaaacatgctgggatagagacaggcaaaaaccatctcgcgcaagcgattcagatctttaactggtttcagcaaaacatcttcaacgcccagacgtaacgctttggcaatatctgccatattttcagtggcagatatcaccagaactggggtctggtcgcctctgttacgtatatgctccagcagtttaagcccgttcattcgtggcatcgcgatatcacatatcatcaggtctggagtgaaacctcccagcaactcaagggcatccaccccatcagccgccagtaccgttgtcgctcccaatgaggaaaaccatgaatccagaagcgagcgaaatacctgctcatcttcaacaatgagaatctgttttccgaccaatggctgcgtcat

SEQ ID NO: 16 - DNA, 414 bp, Escherichia coli
  source 1..414: mol_type = genomic DNA
atgagcgaagcacttaaaattctgaacaacatccgtactcttcgtgcgcaggcaagagaatgtacacttgaaacgctggaagaaatgctggaaaaattagaagttgttgttaacgaacgtcgcgaagaagaaagcgcggctgctgctgaagttgaagagcgcactcgtaaactgcagcaatatcgcgaaatgctgatcgctgacggtattgacccgaacgaactgctgaatagccttgctgccgttaaatctggcaccaaagctaaacgtgctcagcgtccggcaaaatatagctacgttgacgaaaacggcgaaactaaaacctggactggccaaggccgtactccagctgtaatcaaaaaagcaatggatgagcaaggtaaatccctcgacgatttcctgatcaagcaataa

SEQ ID NO: 17 - DNA, 717 bp, Escherichia coli
  source 1..717: mol_type = genomic DNA
atgcagaccccgcacattcttatcgttgaagacgagttggtaacacgcaacacgttgaaaagtattttcgaagcggaaggctatgatgttttcgaagcgacagatggcgcggaaatgcatcagatcctctctgaatatgacatcaacctggtgatcatggatatcaatctgccgggtaagaacggtcttctgttagcgcgtgaactgcgcgagcaggcgaatgttgcgttgatgttcctgactggccgtgacaacgaagtcgataaaattctcggcctcgaaatcggtgcagatgactacatcaccaaaccgttcaacccgcgtgaactgacgattcgtgcacgcaacctactgtcccgtaccatgaatctgggtactgtcagcgaagaacgtcgtagcgttgaaagctacaagttcaatggttgggaactggacatcaacagccgttcgttgatcggccctgatggcgagcagtacaagctgccgcgcagcgagttccgcgccatgcttcacttctgtgaaaacccaggcaaaattcagtcccgtgctgaactgctgaagaaaatgaccggccgtgagctgaaaccgcacgaccgtactgtagacgtgacgatccgccgtattcgtaaacatttcgaatctacgccggatacgccggaaatcatcgccaccattcacggtgaaggttatcgcttctgcggtgatctggaagattaa

SEQ ID NO: 18 - DNA, 633 bp, Escherichia coli
  source 1..633: mol_type = genomic DNA
ttaacgagtgccgtaaacgacgatggttttaccgtgtgcggagatcaggttctgatcttccagcatcttcagaatgcgtcccacggtttcacgagaacagccgacaatctgaccaatttcctgacgggtaattttgatttgcataccgtccgggtgagtcatagcgtctggttgttttgccagattcagcagagtctgtgcaatgcggcccgtcacgtcgaggaacgccaggttgcccactttctctgaagtgacttgcagacgacgcgccatctgtgcagacaaacgcatcagaatgtccgggtttacctgaatcaattggcgaaattttttgtacgaaatttcagccacttcacaggcggttttcgcacgtacccatgcgctacgttcctggccctcttcaaacaggcccagttcgccaataaaatcaccctgattcagataggagaggatcatttctttaccctcttcgtctttgatcagcactgccacagagcctttaacgatgtagtacagcgtttccgctttttcaccctggtgaataagcgtgctcttggatgggtacttatgaatgtggcaatgagacaagaaccattcgagagtcgggtctgtttgcggtttgccaagcaccat

SEQ ID NO: 19 - DNA, 447 bp, Escherichia coli
  source 1..447: mol_type = genomic DNA
atgactgataacaataccgccctaaagaaagctggcctgaaagtaacgcttcctcgtttaaaaatcctggaagttcttcaggagccggacaaccatcacgtcagtgcggaagatttatacaaacgtctgatcgatatgggtgaagaaattggtctggctacggtatatcgcgtactgaaccagtttgacgacgctggtatcgtcacccgccacaattttgaaggcggtaaatccgtatttgaactgacacagcaacatcaccacgatcacctgatctgcctcgactgcggcaaggttatcgaatttagtgatgattccatcgaagcgcgtcagcgtgaaattgccgcaaaacatggcattcgcctgactaaccacagtctctatctttacggtcactgtgccgaaggcgattgccgcgaagatgaacatgcgcacgaaggcaaataa

SEQ ID NO: 20 - DNA, 396 bp, Escherichia coli
  source 1..396: mol_type = genomic DNA
atgaaatgtccggtttgccaccagggagaaatggtttctggcattaaagatattccatacaccttccgtggacgaaaaacagtattgaaaggtatccacggtttatattgtgtccattgcgaagagagcatcatgaataaagaagagtcagatgctttcatggcgcaagtaaaggcatttcgggcttcggtgaatgccgaaacagtggcacctgaatttatagtgaaggttcgaaaaaagctctctcttacccaaaaagaggcaagcgaaatttttgggggaggtgtaaatgcgttttcgcgttacgaaaaaggcaatgcccaacctcatccttccacaatcaaacttttacgtgttctggataagcatccagaactattgaatgaaatccgttaa

SEQ ID NO: 21 - DNA, 117 bp, Escherichia coli
  source 1..117: mol_type = genomic DNA
gaaacggagcggcacctcttttaacccttgaagtcactgcccgtttcgagagtttctcaactcgaataactaaagccaacgtgaacttttgcggatctccaggatccgctttttttt

SEQ ID NO: 22 - DNA, 87 bp, Escherichia coli
  source 1..87: mol_type = genomic DNA
aaaaaataagcccgtgtaagggagattacacaggctaaggaggtggttcctggtacagctagcattttatgggttatgtttttcagc

SEQ ID NO: 23 - DNA, 203 bp, synthetic construct
  misc_feature 1..203: note = Recombinant promoter sequence PmglB_70UTR_SD8
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 24 - DNA, 203 bp, synthetic construct
  misc_feature 1..203: note = Recombinant promoter element PmglB_70UTR_SD10
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 25 - DNA, 203 bp, synthetic construct
  misc_feature 1..203: note = Recombinant promoter element PmglB_54UTR
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 26 - DNA, 141 bp, synthetic construct
  misc_feature 1..141: note = Recombinant promoter element Plac_70UTR
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 27 - DNA, 203 bp, synthetic construct
  misc_feature 1..203: note = Recombinant promoter element PmglB_70UTR_SD9
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 28 - DNA, 203 bp, synthetic construct
  misc_feature 1..203: note = Recombinant promoter element PmglB_70UTR_SD4
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 29 - DNA, 203 bp, synthetic construct
  misc_feature 1..203: note = Recombinant promoter element PmglB_70UTR_SD5
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 30 - DNA, 310 bp, synthetic construct
  misc_feature 1..310: note = Recombinant promoter element PglpF_SD4
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 31 - DNA, 203 bp, synthetic construct
  misc_feature 1..203: note = Recombinant promoter element PmglB_70UTR_SD7
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 32 - DNA, 203 bp, synthetic construct
  misc_feature 1..203: note = Recombinant promoter element PmglB_70UTR
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 33 - DNA, 189 bp, synthetic construct
  misc_feature 1..189: note = Recombinant promoter element PglpA_70UTR
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 34 - DNA, 239 bp, synthetic construct
  misc_feature 1..239: note = Recombinant promoter element PglpT_70UTR
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 35 - DNA, 300 bp, Escherichia coli
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 36 - DNA, 310 bp, synthetic construct
  misc_feature 1..310: note = Recombinant promoter element PglpF_SD10
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 37 - DNA, 310 bp, synthetic construct
  misc_feature 1..310: note = Recombinant promoter element PglpF_SD5
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 38 - DNA, 310 bp, synthetic construct
  misc_feature 1..310: note = Recombinant promoter element PglpF_SD8
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 39 - DNA, 350 bp, synthetic construct
  misc_feature 1..350: note = Recombinant promoter element PmglB_16UTR
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 40 - DNA, 310 bp, synthetic construct
  misc_feature 1..310: note = Recombinant promoter element PglpF_SD9
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 41 - DNA, 300 bp, synthetic construct
  misc_feature 1..300: note = Recombinant promoter element PglpF_SD7
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 42 - DNA, 310 bp, synthetic construct
  misc_feature 1..310: note = Recombinant promoter element PglpF_SD6
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 43 - DNA, 189 bp, synthetic construct; identical to SEQ ID NO: 33
  misc_feature 1..189: note = Recombinant promoter element PglpA_16UTR
  source 1..189: mol_type = other DNA

SEQ ID NO: 44 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 45 - DNA, 300 bp, synthetic construct
  misc_feature 1..300: note = Recombinant promoter element PglpF_SD3
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 46 - DNA, 300 bp, synthetic construct
  misc_feature 1..300: note = Recombinant promoter element PglpF_SD1
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 47 - protein, 302 aa, Helicobacter pylori
MAFKVVQICGGLGNQMFQYAFAKSLQKHSNTPVLLDITSFDWSDRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYQCKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLIENPEKIIIGPKHWLFGHENILCKEWVKIESHFEVKSQKYNALG

SEQ ID NO: 48 - protein, 425 aa, Helicobacter pylori
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 49 - protein, 342 aa, Neisseria meningitidis
MERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 50 - protein, 483 aa, Photobacterium damselae
MCNSDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 51 - protein, 394 aa, Rosenbergiella nectarea
MQSFTPPAPKGGNPVFMMFMLVTFFVSIAGALQAPTLSLYLSQELAAKPFMVGLFFTINAVTGIIISFILAKRSDRKGDRRRLLMFCCAMAIANALMFAFVRQYVVLITLGLILSALTSVVMPQLFALAREYADRTGREVVMFSSVMRTQMSLAWVIGPPISFALALNYGFITLYLVAAALFLLSLILIKTTLPSVPRLYPAEDLAKSAASGWKRTDVRFLFAASVLMWVCNLMYIIDMPLYISKSLGMPESFAGVLMGTAAGLEIPVMLLAGYLAKRVGKRPLVIVAAVCGLAFYPAMLVFHQQTGLLIIQLLNAVFIGIVAGLVMLWFQDLMPGKAGAATTLFTNSVSTGMIFAGLCQGLLSDLLGHQAIYVLATVLMVIALLLLLRVKEQA

SEQ ID NO: 52 - protein, 396 aa, Serratia marcescens
MQRLSRLSLRINPIFAAFLLIAFLSGIAGALLTPTLSLFLTTEVKVRPLWVGLFYTANAVAGIVVSFLLAKRSDTRGDRRRLILLCCLMAVGNCLLFAFNRDYLTLITAGVLMSAVANTAMPQIFALAREYADSEAREVVMFSSVMRAQLSLAWVIGPPLSFALALNYGFTVMFLIAAVTFAVCVLLVGFMLPSVPRAAENEGLQGGVSAPIAPASAWRNRDVRLLFIASMLMWTCNTLYIIDMPLYITADLGLPEGLAGVLMGTAAGLEIPAMLLAGYYVKRFGKRNMMLLAVVAGVLFYLGLTVLESKPALIALQLLNAVFIGIVAGIGMLYFQDLMPGRPGAATTLFTNSISTGVILAGVLQGALVENLGHGSVYWMAALLALAALGMSAKVR

SEQ ID NO: 53 - protein, 505 aa, Klebsiella pneumoniae
MYKKRKLAILIALLTGTAAAHGQTDLNSIEARLAALEKRLQDAETRASTAESRAASAEQKVQQLTQQQQQTQATTQQVARRTTQLEEKAERPGGFEFHGYARSGVIMNDSAASTKSGAYMTPAGETGGAIGRLGNQADTYVEMNLEHKQTLDNGATTRFKVMVADGQTTYNDWTASSSDLNVRQAFVELGNLPTFEGPFKGSTLWAGKRFDRDNFDIHWIDSDVVFLAGTGGGIYDVKWNDSLRSNFSLYGRNFGDIADSSNSVQNYIVSMNNFAGPVQMMVSGMRAKDNDDRQDANGNLVKGDAANTGVHALLGLHNESFYGLRDGTSKTALLYGHGLGAEVKGIGSDGALRPGANTWRFASYGTTPLSDRWFIAPAVLAQSSKDRYVDGDSYQWATLNLRLIQEVTQNFALAWEGSYQYMDLQPEGYNDRHAVNGSFYKLTFAPTFKVGSIGDFFSRPEIRFYTSWMDWSKKLDNYANDDALGSNGFKSGGEWSFGMQMETWF

SEQ ID NO: 54 - protein, 456 aa, Klebsiella pneumoniae
MDFEQISRSLLPLLGGKENIASAAHCATRLRLVLVDDALADQQAIGKIDGVKGCFRNAGQMQIIFGTGVVNKVYAAFIQAAGISESSKSEAADLAAKKLNPFQRIARLLSNIFVPIIPAIVASGLLMGLLGMVKTYGWVDPSNALYIMLDMCSSAAFIILPILIGFTAAREFGGNPYLGATLGGILTHPALTNAWGVAAGFHTMNFFGIEVAMIGYQGTVFPVLLAVWFMSMVEKRLRRVIPDALDLILTPFLTVIISGFIALLLIGPAGRALGDGISFILSTLISHAGWLAGLLFGGLYSVIVITGIHHSFHAIEAGLLGNPSIGVNFLLPIWAMANVAQGGACFAVWFKTKDAKIKAITLPSAFSAMLGITEAAIFGINLRFVKPFIAALVGGAAGGAWVVSMHVYMTAVGLTAIPGMAIVQASSLLNYIIGMAIAFAVAFALSLTLKYKTDAE

SEQ ID NO: 55 - protein, 466 aa, Salmonella enterica
MSLPSRLPAILQAVMQGQPRALADSHYPRWHHAPVTGLMNDPNGFIEFAGRYHLFYQWNPLACDHTFKCWAHWSSIDLLHWQHEPIALMPDEEYDRNGCYSGSAVDNNGTLTLCYTGNVKFAEGGRTAWQCLATENADGTFRKIGPVLPLPEGYTGHVRDPKVWRHEDLWYMVLGAQDRQKRGKVLLFSSADLHQWTSMGEIAGHGINGLDDVGYMWECPDLFPLGDQHILICCPQGIAREEECYLNTYPAVWMAGEFDYAAGAFRHGELHELDAGFEFYAPQTMLTSDGRRLLVGWMGVPEGEEMLQPTLNNGWIHQMTCLRELEFINGQLYQRPLRELSALRGEANGWSGNALPLAPMEIDLQTRGGDMLSLDFGGVLTLECDASGLRLARRSLASDEMHYRYWRGNVRSLRVFIDQSSVEIFINGGEGVMSSRYFPACSGQLTFSGITPDAFCYWPLRTCMVE

SEQ ID NO: 56 - protein, 334 aa, Salmonella enterica
MKTKRVTIKDIAEQAGVSKATASLVLNGRGKELRVAQETRERVLSIARKHHYQPSIHARSLRNNRSHTIGLVVPEITNHGFAVFAHELEMLCREAGVQLLISCTDENPGQESVVVNNMIARQVDGMIVASCMHNDADYLKLSQQLPVVLFDRCPNESALPLVMTDSITPTAELISRIAPQHSDEFWFLGGQARLSPSRDRLTGFTQGLAQAGIALRPEWVINGNYHPSSGYEMFAALCARLGRPPKALFTAACGLLEGVLRYMSQHHLLDSDIHLTSFDDHYLYDSLSLRIDTVQQDNRQLAWHCYDLISQLIEGDTPETLQRYLPATLQFRHQ

SEQ ID NO: 57 - DNA, 6706 bp, Escherichia coli
  source 1..6706: mol_type = other DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 58 - DNA, 2818 bp, Campylobacter jejuni
  source 1..2818: mol_type = other DNA
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

SEQ ID NO: 59 - protein, 346 aa, Campylobacter jejuni
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 60 - protein, 372 aa, Campylobacter jejuni
VKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 61 - protein, 221 aa, Campylobacter jejuni
MSLAIIPARGGSKGIKNKNLVLLNNKPLIYYTIKAALNAKSISKVVVSSDSDEILNYAKSQNVDILKRPISLAQDDTTSDKVLLHALKFYKDYEDVVFLQPTSPLRTNIHINEAFNLYKNSNANALISVSECDNKILKAFVCNDCGDLAGICNDEYPFMPRQKLPKTYMSNGAIYILKIKEFLNNPSFLQSKTKHFLMDESSSLDIDCLEDLKKVEQIWKK

SEQ ID NO: 62 - DNA, 20 bp, synthetic construct
  misc_feature 1..20: note = RT-qPCR forward primer for lacY
  source 1..20: mol_type = other DNA
gggagcctacttcccgtttt

SEQ ID NO: 63 - DNA, 20 bp, synthetic construct
  misc_feature 1..20: note = RT-qPCR reverse primer for lacY
  source 1..20: mol_type = other DNA
gcccgagtttgtcagaaagc

SEQ ID NO: 64 - DNA, 20 bp, synthetic construct
  misc_feature 1..20: note = RT-qPCR forward primer for futC
  source 1..20: mol_type = other DNA
cggttgtcagctgggtattg

SEQ ID NO: 65 - DNA, 21 bp, synthetic construct
  misc_feature 1..21: note = RT-qPCR reverse primer for futC
  source 1..21: mol_type = other DNA
cgcgtggtcatatccatgaac

SEQ ID NO: 66 - DNA, 20 bp, synthetic construct
  misc_feature 1..20: note = RT-qPCR forward primer for manA
  source 1..20: mol_type = other DNA
gccattttgttctgcgtcga

SEQ ID NO: 67 - DNA, 19 bp, synthetic construct
  misc_feature 1..19: note = RT-qPCR reverse primer for manA
  source 1..19: mol_type = other DNA
gcctttgacagtcaccggt

SEQ ID NO: 68 - DNA, 20 bp, synthetic construct
  misc_feature 1..20: note = RT-qPCR forward primer for manB
  source 1..20: mol_type = other DNA
gcccaccattacttccgtga

SEQ ID NO: 69 - DNA, 20 bp, synthetic construct
  misc_feature 1..20: note = RT-qPCR reverse primer for manB
  source 1..20: mol_type = other DNA
ctgttgatctcaccgcttgc

SEQ ID NO: 70 - DNA, 20 bp, synthetic construct
  misc_feature 1..20: note = RT-qPCR forward primer for gmd
  source 1..20: mol_type = other DNA
cgtccggctgaagttgaaac

SEQ ID NO: 71 - DNA, 20 bp, synthetic construct
  misc_feature 1..20: note = RT-qPCR reverse primer for gmd
  source 1..20: mol_type = other DNA
cgcttcgaggtcattagcca

SEQ ID NO: 72 - DNA, 20 bp, synthetic construct
  misc_feature 1..20: note = RT-qPCR forward primer for nec
  source 1..20: mol_type = other DNA
gggtctggttatgctgtggt

SEQ ID NO: 73 - DNA, 20 bp, synthetic construct
  misc_feature 1..20: note = RT-qPCR reverse primer for nec
  source 1..20: mol_type = other DNA
cgtaaatcgcttggtgaccc

SEQ ID NO: 74 - DNA, 20 bp, synthetic construct
  misc_feature 1..20: note = RT-qPCR forward primer for scrY
  source 1..20: mol_type = other DNA
ccctcaacctgcgtctgatt

SEQ ID NO: 75 - DNA, 20 bp, synthetic construct
  misc_feature 1..20: note = RT-qPCR reverse primer for scrY
  source 1..20: mol_type = other DNA
tagaagctgccattgaccgc

SEQ ID NO: 76 - DNA, 20 bp, synthetic construct
  misc_feature 1..20: note = RT-qPCR forward primer for crp
  source 1..20: mol_type = other DNA
cagacgctatgactcacccg

SEQ ID NO: 77 - DNA, 20 bp, synthetic construct
  misc_feature 1..20: note = RT-qPCR reverse primer for crp
  source 1..20: mol_type = other DNA
tgtgcggagatcaggttctg

SEQ ID NO: 78 - DNA, 20 bp, synthetic construct
  misc_feature 1..20: note = RT-qPCR forward primer for cra
  source 1..20: mol_type = other DNA
aaggtttccgtactgcctgg

SEQ ID NO: 79 - DNA, 20 bp, synthetic construct
  misc_feature 1..20: note = RT-qPCR reverse primer for cra
  source 1..20: mol_type = other DNA
atcactccttgcaacaacgc

SEQ ID NO: 80 - DNA, 20 bp, synthetic construct
  misc_feature 1..20: note = RT-qPCR forward primer for cysG
  source 1..20: mol_type = other DNA
gtattccactcacgcatcgc

SEQ ID NO: 81 - DNA, 20 bp, synthetic construct
  misc_feature 1..20: note = RT-qPCR reverse primer for cysG
  source 1..20: mol_type = other DNA
atagtcgcggcctgattcaa

SEQ ID NO: 82 - DNA, 250 bp, Escherichia coli
  source 1..250: mol_type = other DNA
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgaaatcgaaaacaacaaggaggaaacagct

SEQ ID NO: 83 - DNA, 182 bp, Escherichia coli
  source 1..182: mol_type = other DNA
gaaaacattcataaattaaatgtgaattgccgcacacattattaaataagatttacaaaatgttcaaaatgacgcatgaaatcacgtttcactttcgaattatgagcgaatatgcgcgaaatcaaacaattcatgtttttactatggctaaatggtaaaaaacgaacaaggaggaaacagct

SEQ ID NO: 84 - DNA, 350 bp, Escherichia coli; identical to SEQ ID NO: 39
  source 1..350: mol_type = other DNA

SEQ ID NO: 85 - protein, 417 aa, Escherichia coli [sequence omitted]

SEQ ID NO: 86 - protein, 387 aa, Rouxiella badensis
MSSRRLSIIFATFLLVSFLTGIAGALQAPTLSLFLTNEVKVRPLWVGLFYTVNALGGIVISFLLANYSDKKGDRRKLLFFCTLMAIGNSLIFAYSRDYLVLISVGVLLAAIGNASMPQLFALAREYADRSAHEVVMFSSMMRATLSLAWVLGPPISFTLALNYGFTLMYLCAAGVFIFSALMVWFFLPSVGRIEQPVDKVVVHVSAWKNRDVRLLFFASLLMWTCNIMYIIDMPLYITSDLGLPEGLAGLLMGAAAGLEIPVMLIAGYLVKRTGKRRLMLCAAVFGILFYLGLVLFQFKAALMILQLFNAIFIGIIAGIGMLYFQDLMPGRAGSATTLFTNSISTGAILAGVIQGTIVQNFGHYQVYWMALALAVGALVLMTRVKNV

SEQ ID NO: 87 - protein, 393 aa, Yersinia bercovieri
MKSALTFSRRINPVFLAFFVVAFLSGIAGALQAPTLSLFLSTEVKVRPLWVGLFYTVNAIAGITVSFILAKRSDSRGDRRKLIMVCYLMAVGNCLLFAFNRDYLTLITAGVLLASVANTAMPQIFALAREYADSSAREVVMFSSIMRAQLSLAWVIGPPLSFMLALNYGFTLMFSIAAGIFVLSALVVWFILPSVPRAEPVVDAPVVVQGSLFADKNVLLLFIASMLMWTCNTMYIIDMPLYITASLGLPERLAGLLMGTAAGLEIPIMLLAGYSVRYFGKRKIMLFAVLAGVLFYTGLVLFKFKTALMLLQIFNAIFIGIVAGIGMLYFQDLMPGRAGAATTLFTNSISTGVILAGVLQGGLTETWGHDSVYVMAMVLSILALIICARVREA

SEQ ID NO: 88 - protein, 393 aa, Yersinia frederiksenii
MKSALTFSRRINPVFLAFFVVAFLSGIAGALQAPTLSLFLSTEVKVRPLWVGLFYTVNAIAGITVSFVLAKRSDLRGDRRKLILVCYLMAVGNCLLFAFNRDYLTLITAGVLLAAVANTAMPQIFALAREYADNSAREVVMFSSIMRAQLSLAWVIGPPLSFMLALNYGFTLMFCIAAGIFVLSALVVWFILPSVQRAEPVMDAPTVAQGSLFADKDVLLLFIASMLMWTCNTMYIIDMPLYITASLGLPERLAGLLMGTAAGLEIPIMLLAGYSVRRFGKRKIMLFAVLAGVLFYTGLVLFKFKSALMLLQIFNAIFIGIVAGIGMLYFQDLMPGRAGAATTLFTNSISTGVILAGVLQGVLTETWGHNSVYVMAMILAILSLIICARVREA

SEQ ID NO: 89 - protein, 392 aa, Pantoea vagans
MKSLLTRKRRINPVFLAFMAASFMIGVAGALQAPTLSLFLTREVQARPLWVGLFFTVNAIAGIVVSMLVAKRSDSRGDRRTLILFCCAMAFCNALLFAFTRHYLTLITLGVLLSALASVSMPQIFALAREYADQSAREAVMFSSVMRAQLSLAWVIGPPLSFALALNFGFVTLFLVAAALFLVCILLIKFTLPSVPRAEPLMRSGGMPLSGWRDRDVRLLFIASVTMWTCNTMYIIDMPLYISVTLGLPEKLAGLLMGTAAGLEIPVMLLAGHYAKRVGKRNLMLIAVAAGVLFYAGLAMFASQTALMALQLFNAVFIGIIAGIGMLWFQDLMPGRPGAATTMFTNSISTGMILAGVIQGTLSERFGHIAVYWLALGLAVAAFAMSARVKNV

SEQ ID NO: 90 - protein, 330 aa, Escherichia coli
MSQNTLKVHDLNEDAEFDENGVEVFDEKALVELEPSDNDLAEEELLSQGATQRVLDATQLYLGEIGYSPLLTAEEEVYFARRALRGDVASRRRMIESNLRLVVKIARRYGNRGLALLDLIEEGNLGLIRAVEKFDPERGFRFSTYATWWIRQTIERAIMNQTRTIRLPIHIVKELNVYLRTARELSHKLDHEPSAEEIAEQLDKPVDDVSRMLRLNERITSVDTPLGGDSEKALLDILADEKENGPEDTTQDDDMKQSIVKWLFELNAKQREVLARRFGLLGYEAATLEDVGREIGLTRERVRQIQVEGLRRLREILQTQGLNIEALFRE

SEQ ID NO: 91 - protein, 613 aa, Escherichia coli
MEQNPQSQLKLLVTRGKEQGYLTYAEVNDHLPEDIVDSDQIEDIIQMINDMGIQVMEEAPDADDLMLAENTADEDAAEAAAQVLSSVESEIGRTTDPVRMYMREMGTVELLTREGEIDIAKRIEDGINQVQCSVAEYPEAITYLLEQYDRVEAEEARLSDLITGFVDPNAEEDLAPTATHVGSELSQEDLDDDEDEDEEDGDDDSADDDNSIDPELAREKFAELRAQYVVTRDTIKAKGRSHATAQEEILKLSEVFKQFRLVPKQFDYLVNSMRVMMDRVRTQERLIMKLCVEQCKMPKKNFITLFTGNETSDTWFNAAIAMNKPWSEKLHDVSEEVHRALQKLQQIEEETGLTIEQVKDINRRMSIGEAKARRAKKEMVEANLRLVISIAKKYTNRGLQFLDLIQEGNIGLMKAVDKFEYRRGYKFSTYATWWIRQAITRSIADQARTIRIPVHMIETINKLNRISRQMLQEMGREPTPEELAERMLMPEDKIRKVLKIAKEPISMETPIGDDEDSHLGDFIEDTTLELPLDSATTESLRAATHDVLAGLTAREAKVLRMRFGIDMNTDYTLEEVGKQFDVTRERIRQIEAKALRKLRHPSRSEVLRSFLDD

SEQ ID NO: 92 - protein, 133 aa, Escherichia coli
MTLPSGHPKSRLIKKFTALGPYIREGKCKDNRFFFDCLAVCVNVKPAPEVREFWGWWMELEAQESRFTYSYQFGLFDKAGDWKSVPVKDTEVVERLEHTLREFHEKLRELLTTLNLKLEPADDFRDEPVKLTA

SEQ ID NO: 93 - DNA, 789 bp, Bacillus subtilis
  source 1..789: mol_type = genomic DNA
atgacacaaccatcaaaaactacgaaactaactaaagatgaagtcgatcggctcataagcgattaccaaacaaagcaagatgaacaagcgcaggaaacgcttgtgcgggtgtatacaaatctggttgacatgcttgcgaaaaaatactcaaaaggcaaaagcttccacgaggatctccgccaggtcggcatgatcgggctgctaggcgcgattaagcgatacgatcctgttgtcggcaaatcgtttgaagcttttgcaatcccgacaatcatcggtgaaattaaacgtttcctcagagataaaacatggagcgttcatgtgccgagacgaattaaagaactcggtccaagaatcaaaatggcggttgatcagctgaccactgaaacacaaagatcgccgaaagtcgaagagattgccgaattcctcgatgtttctgaagaagaggttcttgaaacgatggaaatgggcaaaagctatcaagccttatccgttgaccacagcattgaagcggattcggacggaagcactgtcacgattcttgatatcgtcggatcacaggaggacggatatgagcgggtcaaccagcaattgatgctgcaaagcgtgcttcatgtcctttcagaccgtgagaaacaaatcatagaccttacgtatattcaaaacaaaagccaaaaagaaactggggacattctcggtatatctcaaatgcacgtctcgcgcttgcaacgcaaagctgtgaagaagctcagagaggccttgattgaagatccctcgatggagttaatgtaa

SEQ ID NO: 94 - protein, 262 aa, Bacillus subtilis
MTQPSKTTKLTKDEVDRLISDYQTKQDEQAQETLVRVYTNLVDMLAKKYSKGKSFHEDLRQVGMIGLLGAIKRYDPVVGKSFEAFAIPTIIGEIKRFLRDKTWSVHVPRRIKELGPRIKMAVDQLTTETQRSPKVEEIAEFLDVSEEEVLETMEMGKSYQALSVDHSIEADSDGSTVTILDIVGSQEDGYERVNQQLMLQSVLHVLSDREKQIIDLTYIQNKSQKETGDILGISQMHVSRLQRKAVKKLREALIEDPSMELM

SEQ ID NO: 95 - DNA, 1119 bp, Bacillus subtilis
  source 1..1119: mol_type = genomic DNA
atggcagagaaaccgttacgcccattggcagaaggtgaactttccatcgatcaagtaaaggaacaattggtggagttagggaaaaagcgtggcgttctcacttatgctgagatcacggaaaagctagctccatacgatcaagactcagaccaaatggacgagttttttgagtatctaggtgaacagggggtcgagattttaaacgataatgaagaggttccaagcttgcaacaagtagagaaggaagaagaggagtttgacttaaacgacttgagtgtacctccaggtgttaagattaacgatcctgttcgcatgtatttgaaggaaattggtcgtgtcccgctattaacggccgaagaagaaattgaactagctacgcgaattgagcagggagatgaagaagcgaagcgtcgcttagctgaagcgaacttacgtctcgttgttagtatcgccaaacgttacgtcggtcgtggcatgctgtttcttgaccttattcaagaaggtaacatgggactaattaaagcggtagaaaaatttgattataacaagggctttaagttcagtacgtacgctacatggtggatcagacaagcaattacacgcgcaattgccgaccaagcacggacgatccgtatcccggtccatatggtggaaacgatcaataagttaatacgtgttcagcgtcagcttctccaggatgtaggtcgtgagccatcacctgaggaagttgcggaagagatggatttaacacctgagaaagttcgagagattcttaaaatcgctcaggagcctgtttcgctcgagactccgataggcgaagaggatgattctcacctaggagattttattgaagatcaagatgcgttagctccttcagatgcggctgcttatgagttattaaaagagcagctagaagatgttcttgacacgttgactgatcgtgaagaaaatgttctccgccttcgctttgggcttgatgatgggcgcacacgtacgttagaagaagtagggaaggtctttggcgtcactcgagagcgcatccgccaaattgaggcgaaagcgttacgtaaacttagacacccaagccgtagtaaacggttgaaggacttcttagaataa

SEQ ID NO: 96 - protein, 372 aa, Bacillus subtilis
MAEKPLRPLAEGELSIDQVKEQLVELGKKRGVLTYAEITEKLAPYDQDSDQMDEFFEYLGEQGVEILNDNEEVPSLQQVEKEEEEFDLNDLSVPPGVKINDPVRMYLKEIGRVPLLTAEEEIELATRIEQGDEEAKRRLAEANLRLVVSIAKRYVGRGMLFLDLIQEGNMGLIKAVEKFDYNKGFKFSTYATWWIRQAITRAIADQARTIRIPVHMVETINKLIRVQRQLLQDVGREPSPEEVAEEMDLTPEKVREILKIAQEPVSLETPIGEEDDSHLGDFIEDQDALAPSDAAAYELLKEQLEDVLDTLTDREENVLRLRFGLDDGRTRTLEEVGKVFGVTRERIRQIEAKALRKLRHPSRSKRLKDFLE

SEQ ID NO: 97 - protein, 151 aa, Escherichia coli
MQEGQNRKTSSLSILAIAGVEPYQEKPGEEYMNEAQLAHFRRILEAWRNQLRDEVDRTVTHMQDEAANFPDPVDRAAQEEEFSLELRNRDRERKLIKKIEKTLKKVEDEDFGYCESCGVEIGIRRLEARPTADLCIDCKTLAEIREKQMAG

SEQ ID NO: 98 - protein, 274 aa, Escherichia coli
MQSLHGNCLIAYARHKYILTMVNGEYRYFNGGDLVFADASQIRVDKCVENFVFVSRDTLSLFLPMLKEEALNLHAHKKVSSLLVHHCSRDIPVFQEVAQLSQNKNLRYAEMLRKRALIFALLSVFLEDEHFIPLLLNVLQPNMRTRVCTVINNNIAHEWTLARIASELLMSPSLLKKKLREEETSYSQLLTECRMQRALQLIVIHGFSIKRVAVSCGYHSVSYFIYVFRNYYGMTPTEYQERSAQRLSNRDSAASIVAQGNFYGTDRSAEGIRL

SEQ ID NO: 99 - protein, 629 aa, Escherichia coli
MAEFETTFADLGLKAPILEALNDLGYEKPSPIQAECIPHLLNGRDVLGMAQTGSGKTAAFSLPLLQNLDPELKAPQILVLAPTRELAVQVAEAMTDFSKHMRGVNVVALYGGQRYDVQLRALRQGPQIVVGTPGRLLDHLKRGTLDLSKLSGLVLDEADEMLRMGFIEDVETIMAQIPEGHQTALFSATMPEAIRRITRRFMKEPQEVRIQSSVTTRPDISQSYWTVWGMRKNEALVRFLEAEDFDAAIIFVRTKNATLEVAEALERNGYNSAALNGDMNQALREQTLERLKDGRLDILIATDVAARGLDVERISLVVNYDIPMDSESYVHRIGRTGRAGRAGRALLFVENRERRLLRNIERTMKLTIPEVELPNAELLGKRRLEKFAAKVQQQLESSDLDQYRALLSKIQPTAEGEELDLETLAAALLKMAQGERTLIVPPDAPMRPKREFRDRDDRGPRDRNDRGPRGDREDRPRRERRDVGDMQLYRIEVGRDDGVEVRHIVGAIANEGDISSRYIGNIKLFASHSTIELPKGMPGEVLQHFTRTRILNKPMNMQLLGDAQPHTGGERRGGGRGFGGERREGGRNFSGERREGGRGDGRRFSGERREGRAPRRDDSTGRRRFGGDA

SEQ ID NO: 100 - protein, 337 aa, Escherichia coli
MTQPLVGKQILIVEDEQVFRSLLDSWFSSLGATTVLAADGVDALELLGGFTPDLMICDIAMPRMNGLKLLEHIRNRGDQTPVLVISATENMADIAKALRLGVEDVLLKPVKDLNRLREMVFACLYPSMFNSRVEEEERLFRDWDAMVDNPAAAAKLLQELQPPVQQVISHCRVNYRQLVAADKPGLVLDIAALSENDLAFYCLDVTRAGHNGVLAALLLRALFNGLLQEQLAHQNQRLPELGALLKQVNHLLRQANLPGQFPLLVGYYHRELKNLILVSAGLNATLNTGEHQVQISNGVPLGTLGNAYLNQLSQRCDAWQCQIWGTGGRLRLMLSAE

SEQ ID NO: 101 - protein, 137 aa, Escherichia coli
MSEALKILNNIRTLRAQARECTLETLEEMLEKLEVVVNERREEESAAAAEVEERTRKLQQYREMLIADGIDPNELLNSLAAVKSGTKAKRAQRPAKYSYVDENGETKTWTGQGRTPAVIKKAMDEQGKSLDDFLIKQ

SEQ ID NO: 102 - protein, 238 aa, Escherichia coli
MQTPHILIVEDELVTRNTLKSIFEAEGYDVFEATDGAEMHQILSEYDINLVIMDINLPGKNGLLLARELREQANVALMFLTGRDNEVDKILGLEIGADDYITKPFNPRELTIRARNLLSRTMNLGTVSEERRSVESYKFNGWELDINSRSLIGPDGEQYKLPRSEFRAMLHFCENPGKIQSRAELLKKMTGRELKPHDRTVDVTIRRIRKHFESTPDTPEIIATIHGEGYRFCGDLED

SEQ ID NO: 103 - protein, 210 aa, Escherichia coli
MVLGKPQTDPTLEWFLSHCHIHKYPSKSTLIHQGEKAETLYYIVKGSVAVLIKDEEGKEMILSYLNQGDFIGELGLFEEGQERSAWVRAKTACEVAEISYKKFRQLIQVNPDILMRLSAQMARRLQVTSEKVGNLAFLDVTGRIAQTLLNLAKQPDAMTHPDGMQIKITRQEIGQIVGCSRETVGRILKMLEDQNLISAHGKTIVVYGTR

SEQ ID NO: 104 - protein, 148 aa, Escherichia coli
MTDNNTALKKAGLKVTLPRLKILEVLQEPDNHHVSAEDLYKRLIDMGEEIGLATVYRVLNQFDDAGIVTRHNFEGGKSVFELTQQHHHDHLICLDCGKVIEFSDDSIEARQREIAAKHGIRLTNHSLYLYGHCAEGDCREDEHAHEGK

SEQ ID NO: 105 - protein, 131 aa, Escherichia coli
MKCPVCHQGEMVSGIKDIPYTFRGRKTVLKGIHGLYCVHCEESIMNKEESDAFMAQVKAFRASVNAETVAPEFIVKVRKKLSLTQKEASEIFGGGVNAFSRYEKGNAQPHPSTIKLLRVLDKHPELLNEIR

SEQ ID NO: 106 - DNA, 681 bp, Escherichia coli
  source 1..681: mol_type = genomic DNA
atgaaccccatcgtaattaatcggcttcaacggaagctgggctacacttttaatcatcaggaactgttgcagcaggcattaactcatcgtagtgccagcagtaaacataacgagcgtttagaatttttaggcgactctattctgagctacgttatcgccaatgcgctttatcaccgtttccctcgtgtggatgaaggcgatatgagccggatgcgcgccacgctggtccgtggcaatacgctggcggaactggcgcgcgaatttgagttaggcgagtgcttacgtttagggccaggtgaacttaaaagcggtggatttcgtcgtgagtcaattctcgccgacaccgtcgaagcattaattggtggcgtattcctcgacagtgatattcaaaccgtcgagaaattaatcctcaactggtatcaaactcgtttggacgaaattagcccaggcgataaacaaaaagatccgaaaacgcgcttgcaagaatatttgcagggtcgccatctgccgctgccgacttatctggtagtccaggtacgtggcgaagcgcacgatcaggaatttactatccactgccaggtcagcggcctgagtgaaccggtggttggcacaggttcaagccgtcgtaaggctgagcaggctgccgccgaacaggcgttgaaaaaactggagctggaatga

SEQ ID NO: 107 - protein, 226 aa, Escherichia coli
MNPIVINRLQRKLGYTFNHQELLQQALTHRSASSKHNERLEFLGDSILSYVIANALYHRFPRVDEGDMSRMRATLVRGNTLAELAREFELGECLRLGPGELKSGGFRRESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 108 - DNA, 291 bp, synthetic construct
  misc_feature 1..291: note = Synthetic promoter element PgatY_70UTR
  source 1..291: mol_type = other DNA
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgtgcctacaagcatcgtggaggtccgtgactttcacgcatacaacaaacattaaccaaggaggaaacagct

SEQ ID NO: 109 - DNA, 291 bp, synthetic construct
  misc_feature 1..291: note = Synthetic promoter element PgatY_54UTR
  source 1..291: mol_type = other DNA
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgtgcctacaagcatcgtggaggtccgtgactttcacgcatacaacaaacattaacacgacaggatatgaaa

SEQ ID NO: 110 - DNA, 266 bp, synthetic construct
  misc_feature 1..266: note = Synthetic promoter element PgatY_16UTR
  source 1..266: mol_type = other DNA
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgaaatcgaaaacaaacgacaggatatgaaacaaggaggaaacagct

SEQ ID NO: 111 - DNA, 1176 bp, Escherichia coli
  source 1..1176: mol_type = genomic DNA
ttacagcttgttgtaaacacgcgctaaacggccgtggcctttgacagtcaccggtgattcgttggcggcaataaacgctgattcacccggtttaagctgtaactgctgagaacctttccacaacgttgcatcgccttcgacgcagaacaaaatggcggcactctgctggctaatggtggtttctttatcactaaggtcatgcagcgagaaggcaaaatcatccactggaatcgggaagtccagttctgcaccttgtttcaccggctgggtcaacaactggttagccggtttggcttcgaatttcacattggcaaccagttccggaatatcaatgtatttaggcgtcagacccgcacgcagcacgttatcggagtttgccatcacttccagcgccacgccttgcaggtaagcgtgcggtgtttcagcgaacaggaacatcgcttcgccagggttcaatttcaccacattcagcaatagcggggagaacagaccgctgtcttccgggtaaaattcagaaattaaacgaatcgtttgccacggttcaccctgctggctatcgagggccgattttaaaatcgccagcgcgcgggatttttcttcaccctgcatattcaacaggctggcgaacagttcgcttaaacgttcggcatcaggctgttgtaaaaagtgagcaatcgccggatgtgcacctgcgaccggctggagtagggagacaatctcggaaaattcacgaaacgcgttcatcgcaaggaaaggcgtcagcgcaaaaaccagctccggcttgtggttaggatctttatagttacgctcggcggcatccatcgggatacctgcggcattttctttggcaaaaccgatttcagaattgtgtttgtttggatgaacctgaatggagagtggctgtgctgcgcataatactttgaacaggaaaggcagttcgccaaagcgtttggcaacggcctctccgagcagagtcgatttatcactctcaatcacatcacgcagtgaaacgatatctccggcggcattctgcactcgtgaactgcttttcggatgtgcgcccatccacagctcggccatcggctggctggacggattttccataccataaagttcagtcaacgccgttttgctgccccaggcatagttttgcactgagttaatgagtttttgcat

SEQ ID NO: 112 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 113 - DNA, 1371 bp, Escherichia coli
  source 1..1371: mol_type = genomic DNA
atgaaaaaattaacctgctttaaagcctatgatattcgcgggaaattaggcgaagaactgaatgaagatattgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 114 - protein, 456 aa, Escherichia coli
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

(19) United States
(12) Patent Application Publication
(10) Pub. No.: US 2026/0015637 A1
(43) Pub. Date: Jan. 15, 2026

(54) SIGMA FACTOR MODIFICATIONS FOR BIOSYNTHETIC PRODUCTION

(71) Applicant: DSM IP ASSETS B.V., Maastricht (NL)

(72) Inventors: Margit PEDERSEN, Roskilde (DK); Rugile LABUNSKAITE, Frederiksværk (DK)

(21) Appl. No.: 18/873,822

(22) PCT Filed: Jun. 9, 2023
(86) PCT No.: PCT/EP2023/065491

§ 371 Date: Dec. 11, 2024

(30) Foreign Application Priority Data
Jun. 13, 2022 (DK) PA202200561

(51) Int. Cl. C12P 19/04; C12N 9/10; C12N 15/52; C12N 15/70; C12P 19/18

(52) U.S. Cl. CPC C12P 19/04; C12N 9/1051; C12N 15/52; C12N 15/70; C12P 19/18; C12N 2800/101

(57) ABSTRACT

The present invention relates to the biosynthetic production of heterologous products in a genetically engineered cell comprising heterologous nucleic acids encoding a heterologous product and/or a polypeptide required for the production of the heterologous products, wherein the expression of the growth phase and/or stationary phase RNA polymerase sigma factors in said cell is modulated.

### FIELD

The field of the present invention relates to the biosynthetic production of heterologous products in a genetically engineered cell, wherein the cell has a reduced or abolished functionality to enter stationary phase for instance by reducing and/or abolishing the functionality of a RNA polymerase sigma factor active in the stationary phase of the cell, such as sigma factor S (RpoS) in gram-negative bacteria, or a similar stress response factors in other cells, such as sigma factor B (SigB) in gram-positive bacteria.

### BACKGROUND

The design and construction of bacterial cell factories to produce heterologous products, especially for more complex heterologous products, is of paramount importance to provide innovative and scalable solutions for the more complex products of tomorrow.

To this end, rational strain engineering principles are commonly applied to single bacterial cells. Such principles usually refer to a) the manipulation or introduction of a desired biosynthetic pathway in the host, b) increasing the cellular pools of relevant precursors and intermediates required as donors and/or acceptors in desired enzymatic reactions, c) optimizing the import and export of produced products and/or intermediates by enhancing native import/export systems or by introduction of heterologous transporters into the production cell and d) introduction of genes encoding the heterologous product or introduction of enzymes capable of producing the heterologous product in part or in full.

In the biosynthetic production of a heterologous product, using a genetically engineered cell, expression levels of various polypeptides, such as enzymes, transporters, transcription factors etc., may cause bottlenecks/constrictions in the production. In particular, in the production of heterologous products that require the interplay of multiple enzymes within the cell, as is the case in for example HMO production (see e.g., Yu et al 2021 ACS Synth. Biol. 10, 923-938 and Bych et al 2019 Current Opinion in Biotechnology 56:130-137), the balancing of the expression of the individual enzymes is important. Increasing e.g., the expression of the relevant glycosyl transferases by increasing the copy number of individual genes can benefit the yield of the final products, as can the increase in expression of enzymes involved in the formation of donors for the enzymatic processes or proteins involved in substrate uptake or product export.

A major challenge in the scale-up of aerobic fermentation processes is heterogeneity caused by inefficient mixing. Perfect mixing in large scale bioreactors from approximately 1000 L and more is not feasible, hence, gradients of substrate (Bylund et al. 1998 Bioprocess Engineering, 18 (3), 171-180), nutrients, pH and dissolved gases can be formed. In such conditions, the culture is exposed to various harsh conditions and heterogeneity. E.g., such glucose gradients were shown to trigger E. coli's overflow metabolism at the high glucose concentration zones (Lara et al. 2009 Biotech Bioengineering 104 (6), 1153-1161), as well as to increase the cells' maintenance requirements, eventually leading to performance loss.

Metabolites generated in aerobic fermentations of different microorganisms due to glucose overflow metabolism are for example ethanol, acetate, glutamate, as well as mixed acids, such as lactate and formate. Elevated levels of overflow metabolites can lead to cellular stress, which in turn causes a termination of the exponential growth phase and stimulation towards the stationary phase. Cellular stress is a general problem that hampers cell growth, induces cellular statis or cell death and in turn limits the product yield of heterologous products.

Thus, avoiding bottlenecks in the enzymatic processes needed to produce the heterologous product as well as avoiding or reducing cellular stress responses is paramount when aiming to enhance the production of heterologous products in industrial-scale biological systems.

The stationary phase RNA polymerase sigma factor (encoded by the rpoS gene in E. coli) regulates the transcription of a set of genes at the onset of stationary phase or carbon starved conditions and other stress conditions in microorganisms such as E. coli, thereby effectively promoting the cell to exit the growth phase.

Inhibition of the rpoS gene in E. coli results in increased transcription and accordingly also expression of genes for most TCA cycle enzymes, including citrate synthase, isocitrate dehydrogenase, malate dehydrogenase, and succinate dehydrogenase. While the acetate production in these ΔrpoS strains is significantly reduced (Choi et al 2012, Metabolic Engineering, 14 477-486), the inability of the mutants to utilize acetate as a secondary carbon source is found to be responsible for low biomass yield and early entry into the stationary phase (Rahman et al., 2006, Biotechnol Bioeng, June 20; 94 (3): 585-95). Furthermore, in addition to an upregulated L-threonine metabolism, pyruvate, phosphoenolpyruvate and oxaloacetate accumulate in ΔrpoS strains during the growth phase.

In WO2008/020654, it is reported that the survival rate of rpoS gene-deficient strains dramatically decreases under acidic conditions, while the growth rate increases. Still, this shift from a cellular stress response to a growth phase is shown to enhance the natural production of acidic products, such as L-glutamic acid.

Qian et al 2009, Biotechnology and Bioengineering, Vol. 104, No. 4:651 report improving the production of the natural product 1,4-diaminobutane (putrescine) in E. coli by deleting rpoS. Choi et al., is another example of enhanced production of a natural product in E. coli by deleting rpoS, wherein the authors use the upregulation of the threonine pathway induced by the deletion of rpoS in E. coli to enhance the production of 1-propanol. (Choi et al 2012,

Metabolic Engineering, 14 477-486). 1-Propanol is naturally produced by some microbial cells like Clostridium sp. and yeast and also by E. coli under certain conditions (Letoffe et al 2017 PLOS Genetics https://doi.org/10.1371/journal.pgen.1006800).

Thus, rpoS gene-deficient strains have been tried for enhanced production of natural products predominantly produced in the stationary phase but have not been seen as a solution for enhanced production of heterologous products in the growth phase of the bacterial cultures. Instead, they have been found to give rise to low biomass yield.

### SUMMARY OF THE INVENTION

The present invention relates to the biosynthetic production of heterologous products using genetically engineered cells with a modified RNA transcription pathway. The invention relates to a genetically engineered cell for the biosynthetic production of one or more heterologous product(s), wherein said cell has a modulated RNA transcription pathway which prolongs the growth phase and inhibits entry into the stationary phase of said cell in response to for example limited carbon fed during fermentation. In that regard, in embodiments, the invention relates to a genetically engineered cell for the biosynthetic production of one or more heterologous product(s), wherein the cell has a reduced and/or abolished functionality to enter the stationary phase, for instance by reducing or abolishing the functionality of a stationary phase RNA polymerase sigma factor, such as RpoS in E. coli or SigB in Bacillus or similar stress response factors in other cells.

Furthermore, in embodiments, the invention relates to a genetically engineered cell for the biosynthetic production of one or more heterologous product(s), wherein the genetically engineered cell comprises at least one heterologous nucleic acid encoding a heterologous product and/or a polypeptide involved in the production of the heterologous product, wherein the transcription and/or expression of said heterologous nucleic acid is regulated by a promoter which is recognized by a sigma factor of the cell, which is active in the cellular growth-phase, and wherein the function of a sigma factor of the cell, which is active in the stationary-phase of the cell, is reduced or abolished. Preferably, the genetically engineered cell has been genetically engineered such that the function of the sigma factor of the cell which is active in the stationary-phase of the cell is reduced or abolished.

The reduced or abolished function of RpoS or SigB, may e.g., be obtained by full or partial inactivation of the gene encoding RpoS or SigB and/or by restriction of the function of one or more factors promoting the RpoS function, e.g., by a reduced or abolished function of one or more of the RpoS promoting factors, such as, but not limited to, one or more RpoS promoting factors selected from the group consisting of ArcZ, DksA, GadX, DsrA, DeaD, RprA and Crl.

On the other hand, the reduced or abolished function of RpoS, may e.g., be obtained by enhancing the function of one or more factors inhibiting the RpoS function, e.g., by an enhanced function of the RpoS inhibitory factors, obtained by e.g., overexpression of said RpoS inhibitory factors, such as, but not limited to, overexpression of one or more RpoS inhibitory factors selected from the group consisting of rssB, RNase III, H-NS, ArcA, CRP, Fur, MqsA, OxyS and CyaR.

To enable production of a heterologous product in the genetically engineered cell of the present invention, the cell may in embodiments further comprise at least one heterologous nucleic acid encoding a heterologous product and/or a polypeptide involved in the production of the heterologous product. In general, transcription of nucleic acid sequences in a cell is initiated by recognition of specific DNA sequences by the RNA polymerase. Specifically certain promoter elements in the DNA sequence are recognized by one or more specific RNA transcription factor(s). The sequence recognition by the RNA polymerases may enhance or reduce the transcription of a gene leading to a reduced or enhanced expression of the gene product. In that regard, in embodiments of the present invention, the expression of the product encoded by the heterologous nucleic acid, according to the present invention, is increased when the functionality of RpoS is reduced. Accordingly, in further embodiments, the heterologous nucleic acid is transcribed in the absence of RpoS. In further embodiments, the expression of endogenous nucleic acid sequences involved in the formation of the heterologous product may be manipulated by the exchange of endogenous promoter elements with recombinant promoter elements. This is referred to herein as “recombinant endogenous gene” or “recombinant endogenous nucleic acid”. In further embodiments, said exchange increase the expression of the endogenous nucleic acid, when the functionality of RpoS is reduced. Alternatively, an additional copy of an endogenous gene may be inserted into the cell, under the control of a recombinant promoter element, which increase the expression of said endogenous nucleic acid, when the functionality of RpoS is reduced.

As mentioned above, RNA polymerase transcription factors, such as, but not limited to, RpoS (also known as σ^{S} or σ38), RpoD (also known as σ^{D} or σ⁷⁰), RpoE (also known as σ^{E} or σ24) and RpoH (also known as σ^{H} or σ32), recognize specific elements upstream of the coding sequence, such as, but not limited to, promoters, which initiates RNA transcription of the DNA sequence.

In that regard, in embodiments of the present invention, the genetically engineered cell comprises at least one heterologous nucleic acid encoding a protein which is involved in the production of the heterologous product, wherein the expression of the heterologous nucleic acid is regulated by a promoter which is recognized by the cellular growth-phase is a housekeeping transcription factor RpoD.

In preferred embodiments, the promoter of the present invention is recognized by the RNA polymerase transcription factor RpoD. In even more preferred embodiments, recognition of the promoter by RpoD, promotes the expression of the at least one heterologous nucleic acid. In embodiments, such promotors comprises the consensus motif TT (G/C/T) (A/T) C (A/G) (n)₁₄₋₁₈ TA (T/A) (A/G) (A/T) T located at the 5′-end region of the heterologous nucleic acid, between 5 and 40 nucleotides upstream of the translation start codon AUG, wherein “n” denotes any nucleotide.

In addition, in embodiments of the present invention, the expression of the at least one heterologous nucleic acid is regulated by a promoter that is positively regulated by cAMP-bound cyclic AMP receptor protein (CRP). Accordingly, in embodiments of the present invention, a promotor of the present invention further comprises one or more motifs that are recognized by CRP, such as but not limited to the consensus motif (A/G) TGAnnnnnn (A/T) CAC, located upstream of the translation start codon AUG, wherein “n” denotes any nucleotide, and wherein said recognition by CRP enhances the expression of the at least one heterologous nucleic acid.

Thus, in embodiments, promoters of the present invention are selected from the group consisting of PglpF, Plac, PmglB_70UTR, PglpA_70UTR and PglpT_70UTR (SEQ ID NOs: 35, 44, 32, 33 and 34, respectively) and variants thereof, such as those listed in table 1.

Regarding the heterologous product, there is in theory no limitation. Combination of a reduced or abolished function of RpoS combined with the correct promoter selection, can be used to enhance the production of any heterologous product, such as small molecules, carbohydrates, oligosaccharides, complex macromolecules and/or polypeptides, such as peptides, proteins, and/or enzymes.

Thus, in one embodiment of the present invention, the heterologous nucleic acid of the present invention encodes the heterologous product and/or a polypeptide to be produced by the genetically engineered cells.

In other embodiments of the present invention, the heterologous nucleic acid of the present invention encodes an enzyme, such as a glycosyl transferase or an enzyme involved in formation of nucleotide-activated sugars, or transporter proteins involved in substrate import, carbon source or exporting the produced heterologous product.

In embodiments of the present invention, at least one heterologous nucleic acid of the present invention comprises one or more genes selected from the group consisting of one or more gene(s) encoding a glycosyltransferase, one or more transporter gene(s), one or more CMP-N-acetylneuraminic acid pathway gene(s), one or more recombinant GDP-fucose pathway gene(s) and one or more nucleic acid(s) encoding the heterologous product to be produced by the genetically engineered cell.

In further embodiments of the invention, the heterologous product is one or more oligosaccharide(s), preferably one or more human milk oligosaccharide(s) (HMO(s)), such as but not limited to HMOs selected from lacto-N-triose II (LNT-II) lacto-N-tetraose (LNT), lacto-N-neotetraose (LNnT), lacto-N-neohexaose (LNnH), para-lacto-N-neohexaose (pLNnH), para-lacto-N-hexaose (pLNH), lacto-N-hexaose (LNH), 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In that regard, genetically engineered cell according to the present invention in embodiments comprises a de novo GDP-fucose pathway and/or a heterologous CMP-N-acetylneuraminic acid pathway.

In further embodiments, the genetically engineered cell according to the present invention comprises and/or overexpresses a biosynthetic pathway for making at least one sugar nucleotide selected from the group consisting of GDP-fucose, glucose-UDP-GIcNAc, UDP-galactose, UDP-glucose, UDP-N-acetylglucosamine, UDP-N-acetylgalactosamine (GlcNAc) and CMP-N-acetylneuraminic acid.

Furthermore, in embodiments of the present invention, the one or more oligosaccharide(s) are fucosylated or sialylated.

In further embodiments of the present invention, the genetically engineered cell according to present invention, which produces one or more fucosylated oligosaccharide(s), further comprises an overexpressed mannose-6-phosphate isomerase (manA) and/or phosphomannomutase (manB) gene, i.e., the manA and/or manB genes is/are overexpressed. In that regard overexpression may be obtained by introduction of additional copies of the genes encoding manA and/or manB into the cell either on the chromosome or on a plasmid. Thus, in embodiments, the genetically engineered cell of the present invention comprises at least two copies of manA and/or at least three copies of manB.

The production of one or more oligosaccharide(s) may also be optimized by an enhanced cellular import/export of one or more substrates, metabolites and/or heterologous products.

Accordingly, in embodiments, wherein the lactose import of the cell of the present invention is enhanced by expression of one or more lactose permease(s), such as but not limited to the lactose permease is LacY, and wherein the nucleic acid sequence encoding LacY is under control of a promoter as disclosed herein.

In addition to import/export of substrates, metabolites and/or heterologous products, genetical modifications that expands the genetically engineered cell's ability to grown on different carbon sources are also considered favorable. Accordingly, in embodiments, the cell further expresses a functional sucrose utilization system, such as a heterologous sucrose utilization system encoded by the scrYA and scrBR operons. A sucrose utilization system enables a cell which is otherwise incapable of utilizing sucrose as carbon sources, to utilize sucrose as an carbon source. A functional sucrose utilization system may be considered favorable in terms of large-scale manufacturing. Accordingly, in embodiments of the present invention the genetically engineered cell comprises a functional sucrose utilization system.

In addition, the import and export of metabolites and/or the heterologous products produced by the cell is enhanced by the expression of specific transporter proteins. Accordingly, in embodiments, the genetically engineered cell of the present invention expresses at least one heterologous major facilitator superfamily (MFS) transporter, such as a heterologous MFS transporter is selected from the group consisting of Nec, YberC, Fred, Bad and Vag.

The production of one or more oligosaccharide(s) may also comprise expression of one or more functional enzymes which enables the production of the heterologous product. In embodiments the genetically engineered cell of the present invention expresses at least one heterologous glycosyltransferase. In further embodiments, the at least one heterologous glycosyltransferase is selected from the group consisting of β-1,3-N-acetyl-glucosaminyltransferase(s), β-1,3-galactosyltransferase(s), β-1,4-galactosyltransferase(s), α-1,2-fucosyltransferase(s), α-1,3-fucosyltransferase, α-2,3-sialyltransferase(s) and α-2,6-sialyltransferase(s).

With regards to the genetically engineered cell of the present invention, there are practically no limitations. In that regard, in embodiments, the genetically engineered cell is procaryote, such as a bacterium. In preferred embodiments, said bacterium is selected from the group consisting of gram-positive bacteria such as Bacillus sp., lactobacillus sp., corynebacterium sp. and Campylobacter sp and gram-negative bacteria such as Escherichia sp., Preferably, the genetically engineered cell is E. coli.

Further functional benefits of a reduced level of RpoS in some embodiments relates to formation of acidic metabolites. Accordingly, in embodiments, the cell has a reduced acetate and/or glutamate formation.

The invention also relates to a method for producing a heterologous product, comprising culturing a genetically engineered cell according to the present invention. In embodiments, the cultivating of the genetically engineered cell is done in the presence of an carbon source selected from the group consisting of glucose, sucrose, fructose, xylose and glycerol. In embodiments of the present invention, the pH during the cultivation is maintained above 6.0, preferably above 6.5. In embodiments, the method relates to production of heterologous product. In further embodiments, the product is a human milk oligosaccharide (HMO). In further embodiments, lactose is added during the cultivation of the genetically engineered cells as a substrate for the HMO formation. In embodiments, the produced human milk oligosaccharide (HMO) is retrieved from the culture medium and/or the genetically engineered cell.

The invention also relates to a genetically engineered cell of the invention for use in the production of a heterologous product, such as for use in the production of an HMO, or such as for use in the production of a polypeptide.

### BRIEF DESCRIPTION OF THE FIGURES

FIG. 1 shows the relative production of HMOs in E. coli production strains containing either a functional sigma factor genes (strain 1) and strains where different sigma factors have been deleted ΔrpoS (strain 2), ΔrpoZ (strain 12) and ΔrpoF (strain 13).

FIG. 2 shows the relative production of HMOs in E. coli production strains containing either a functional rpoS gene (Strain 1, 3, 5, 7, 9), or having the rpoS gene removed (ΔrpoS, Strain 2, 4, 6, 8, 10). The E. coli produces either 2′FL (FIG. 2A), DFL (FIG. 2B), 3FL (FIG. 2C), 3′SL (FIG. 2D), or 6′SL (FIG. 2E).

FIG. 3 shows the relative production of 2′FL in an E. coli 2′FL production strain containing either a functional crl gene (Strain 1) or having the crl gene removed (Δcrl, Strain 11).

FIG. 4 shows the relative production of acetate in E. coli HMO production strains grown overnight in minimal media containing glucose. E. coli produces either DFL (FIG. 4A), 3FL (FIG. 4B), or 6′SL (FIG. 4C).

FIG. 5 shows the relative production of acetate in E. coli 3FL production Strain 5 (+rpoS) and Strain 6 (ΔrpoS) during fermentation. Samples were collected at six different timepoints and the amount of acetate was measured. The values are given relative to Strain 5 at time 21 hours.

FIG. 6 shows the production of glutamate in E. coli 3FL production Strain 5 (+rpoS) and Strain 6 (ΔrpoS) during fermentation.

FIG. 7 shows the relative production of 3FL during fermentation of strain 5 (+rpoS) and strain 6 (ΔrpoS). Samples were collected at six different timepoints and the amount of HMO was determined. The values are normalized to Strain 5 at the end of fermentation.

FIG. 8 shows the relative production of 2′FL during fermentation of strain 1 (+rpoS) and strain 2 (ΔrpoS). Fermentation of Strain 1 and 2 were done in duplicates. Samples were collected at six different timepoints and the amount of HMO was determined. The values are normalized to Strain 1 at the end of fermentation.

FIG. 9 shows the relative transcription between Strain 2 (ΔrpoS) and Strain 1+ (+rpoS) during fermentation (2′FL production shown in FIG. 6). RT-qPCR was done to measure the relative transcription of 19 genes (lacY, manB, manB, futC, gmd, nec, scrY, crp, and cra) at two different timepoints in fermentation (41 and 113 hours).

FIG. 10 shows the relative protein content from heterologous genes expression of futC in an E. coli 2′FL production strain containing a functional rpoS gene (Strain 1, RpoS_pos, full line), and in a 2′FL production strain lacking the rpoS gene (Strain 2, RpoS_neg, dotted line). The scale in the Y-axis is the relative amount of protein in log 2.

FIG. 11 shows the relative protein content of selected endogenous genes in an E. coli 2′FL production strain containing a functional rpoS gene (Strain 1, rpoS_pos, full line), and in a 2′FL production strain lacking the rpoS gene (Strain 2, rpoS_neg, dotted line). FIG. 11A shows ManB protein levels, B shows ManC levels, C shows Gmd levels, D shows Fcl levels, E shows SucA levels, F shows ScrB levels, G shows Suc D levels, H shows GltA levels. The scale in the Y-axis is the relative amount of protein in log 2.

FIG. 12 shows the relative production of 2′FL during fermentation of control strain 2 (ΔrpoS) and ManAB+strain 14 (ΔrpoS, manA+, manB+). The fermentations were performed in duplicate represented as two control strain curves (full line) and two ManAB+strain curves (dotted lines).

### DETAILED DESCRIPTION

Biosynthetic production of heterologous products is a common way of producing chemical and biological products in industrial scale settings. Bacterial pathway engineering is in general used to promote the production of products native to the bacterial cell. The genetically engineering performed to optimize the formation of products naturally produced by the cell, rarely entails inserting heterologous nucleic acid sequences, encoding proteins which are not endogenously present in the cell. In production of heterologous products on the other hand, a combination of pathway engineering and introduction of heterologous genes, which upon expression entails specific functionalities or encodes the desired product, is in general used.

A common issue with bacterial production is the expression of large quantities of heterologous products which may be toxic to the cells due to intracellular product buildup, metabolic overflow, intra-or extracellular acidification or metabolite buildup, all of which may induce cellular stress responses.

To overcome the above-mentioned issues, different strategies may be applied, some involving genetic engineering either to stabilize the host cell under large scale fermentation conditions and/or to increase the yield of the desired product.

In the present invention, it has surprisingly been shown that inhibition of the cellular stress response and stationary phase gene expression by modulation of the stationary phase sigma transcription factor, RpoS, also known as sigma 38 (038) or sigma S, can increase product yields between 30% and 700% for 5 different heterologous HMO products (Example 2, 5 and 6). Furthermore, Example 8 shows a 2.3-fold increase of the heterologous protein product, FutC.

### RNA Polymerase Sigma Factors

Sigma factors are an essential part of the RNA polymerase holoenzyme in bacteria and archaea. At the beginning of the transcribing process, a sigma factor is needed to recognize the genomic location which initiates the promoter-specific transcription. In E. coli the sigma factor system is composed of a housekeeping sigma factor 70 or sigma factor D (rpoD) and six alternative sigma factors RpoN/054 (nitrogen limiting), RpoS/38 (stationary phase), RpoH/o32 (heat shock), RpoF/028 (flagella and chemotaxis), RpoE/o24 (extreme heat stress), 019 (iron transport and metabolism) (transcribed from the genes rpoN, rpoS, rpoH, rpoF, rpoE, fecl respectively) as well as the omega factor RpoZ (assembly of the RNA polymerase holoenzyme). Depending on which sigma factor is present in the RNA polymerase holoenzyme different genes will be transcribed in the cell. Other bacteria and archaea also contain a sigma factor system, where each sigma factor play different roles, as illustrated for the E. coli sigma factors above. In the context of the present invention, when the term RpoD is used it refers to a housekeeping sigma factor, which is active in the cellular growth phase, similarly when the term RpoS is used, it refers to a sigma factor that is primarily active under stress conditions such as starvation and stationary phase.

RNA polymerase sigma factors, such as RpoS (also known as os, sigma factor s or sigma factor 38), RpoD (also known as 70, sigma factor 70 or sigma factor D), RpoE (also known as GE, sigma factor 24) and RpoH (also known as gH, sigma factor H or sigma factor 32), recognize specific elements upstream of the coding sequence, such as promoters, which initiates RNA transcription of the DNA sequence. Accordingly, the cell utilizes RNA polymerase transcription factors to regulate the gene expression based upon external or internal stimuli. A reduction in the function of stationary phase sigma transcription factor RpoS leads to an increase in expression of genes that otherwise would have a reduced expression upon activation of RpoS. The expression of some genes regulated by the growth phase transcription factor, RpoD, also known as sigma 70 (σ⁷⁰) or the housekeeping sigma factor, are decreased in response to cellular stress and entry into the stationary phase due to the exchange of sigma 70 with sigma 38 in the RNA polymerase holoenzyme. In that sense, genes that are transcribed by the RpoD containing RNA polymerase may be downregulated upon enhanced expression of RpoS in the cell. RNA polymerase transcription factors, such as RpoS (also known as os), RpoD (also known as σ⁷⁰), RpoE (also known as GE sigma factor 24) and RpoH (also known as σ^{H} or sigma factor 32), recognize specific elements upstream of the coding sequence, such as promoters, which initiates RNA transcription of the DNA sequence. Accordingly, the cell utilizes RNA polymerase transcription factors to regulate the gene expression based upon external or internal stimuli.

Natural bacterial fermentation follows four phases, namely the lag phase, the growth phase, the stationary phase and the death phase. In industrial fed-batch fermentations the cells grow in two phases, a first phase of exponential cell growth in a culture medium ensured by a carbon source, and a second phase of cell growth in a culture medium run under carbon limitation, where the carbon source is added continuously allowing formation of the product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter. The industrial fermentation will always be stopped before significant cell death occurs. For each phase, and other special cellular condition, specific transcription factors come into play, and promote or reduce the expression of specific genes, based on their sequence specificity. In the current context, the terms “growth phase and/or stationary phase RNA polymerase sigma factors” are used for RNA polymerase sigma factors that are active in the cellular growth-phase and/or stationary phase of the cell.

As the different transcription factors recognize specific consensus sequences in promoters upstream of the start codon of a respective gene to be expressed, individual genes are expressed in different phases of the bacterial growth. Thus, the cell reconfigures the expression pattern depending on the specific cellular state and the expressed transcription factor. For example, following the exponential growth phase, once nutrients in the medium are exhausted or limited, bacterial cultures will exhibit decreased growth rate or enter into stationary phase, which is characterized by equilibrium between the numbers of dividing and dying cells and represents a plateau in the growth curve.

In industrial production, the production of the product often takes place under carbon limiting conditions, which controls the cell growth of the cells, without the cell's actually entering stationary phase. Based on the results of the present invention it may be speculated that this transition from exponential growth to limited growth result in increased formation of RpoS although the cells are not in stationary phase.

The promoter landscape and the transcription factors involved in regulating the transcription is quite complex and assigning specific promoters that are efficient over the entire fermentation process have proven highly complicated and difficult, as the cells in general face very different conditions, once introduced into large-scale fermentation, where some cells might face mechanical stress, others might face anaerobic conditions or excessive sugar inflow or starvation.

Thus, finding promoters that are suitable for expression of genes throughout the fermentation process and under varying fermentation conditions has proven challenging.

In the present disclosure, the inventors solve this problem by combining a) promoters that are recognized by a sigma factor active in the cellular growth-phase (the housekeeping sigma factor) and b) reduction of a stationary phase sigma factor. The solution offers an elegant way to prevent reduction of gene expression from genes transcribed from promoters recognized by the housekeeping sigma factor (RpoD or SigA). A reduction of expression is otherwise seen in the presence of RpoS due to competion between the stationary phase sigma factor (RpoS or SigB) and the housekeeping sigma factor (RpoD or SigA).

The examples clearly demonstrate that the herein for the first-time disclosed approach of using a combined approach of a reduced RpoS level in combination with an RpoD driven promoter leads to an increased yield of production.

### Stationary Phase Transcription Factor

Without being bound by theory, we believe that there are at least three potential mechanisms responsible for the increase in production of the heterologous product produced by the genetically engineered cell of the present invention.

In E. coli, sigma D (RpoD) is the primary sigma factor during exponential growth, which together with the RNA polymerase forms the holoenzyme transcribing most of the genes in growing cells through recognition of a consensus sequence in the promoter of the transcribed genes. Changes away from typical growth conditions, such as exponential growth, towards stationary phase leads to the replacement of Sigma D with Sigma S (RpoS) in the RNA polymerase holoenzyme complex. When this occurs, the RNA polymerase holoenzyme no longer recognizes the same transcription targets, leading to a decline in many RNA and protein levels, including components of the protein synthesis machinery.

It is well known that there is a competition between the house-keeping Sigma factor D and the stationary phase Sigma factor S in the RNA polymerase holoenzyme. Consequently, when Sigma S is removed or the amount of it is reduced in the cell, Sigma factor D will remain in the RNA polymerase holoenzyme and continue transcription of genes essential for normal growth and genes involved in the protein synthesis machinery.

The above is an exemplary description referring to the E. coli sigma factor system, similar systems exist in other procaryotes. In the context of the present invention, it is understood that when referring to RpoD it can be generalized to sigma factors of other species, where the sigma factor is active in the cellular growth-phase and acts as a housekeeping sigma factor, one example of a sigma factor with similar functionality is sigma factor A (SigA) from Bacillus subtilis or other gram-positive bacteria. Likewise, when referring to RpoS it can be generalized to sigma factors of other species, where the sigma factor is a stress response or stationary phase sigma factor, one example is sigma factor B (SigB) from Bacillus subtilis or other gram-positive bacteria (see for example Haldenwang 1995 Microbiological Reviews, March 1995, p. 1-30).

In embodiments of the invention the RpoS sigma factor is encoded by a nucleic acid sequence of SEQ ID NO: 1 or a functional homologue thereof having at least 80%, such as at least 85%, such as at least 90% such as at least 95% or 100% sequence identity to SEQ ID NO: 1.

In embodiments of the invention the RpoS sigma factor comprises the amino acid sequence of SEQ ID NO: 90 or a functional homologue thereof having at least 80%, such as at least 85%, such as at least 90% such as at least 95% or 100% sequence identity to SEQ ID NO: 90.

In embodiments of the invention the SigB sigma factor is encoded by a nucleic acid sequence of SEQ ID NO: 93 or a functional homologue thereof having at least 80%, such as at least 85%, such as at least 90% such as at least 95% or 100% sequence identity to SEQ ID NO: 93.

In embodiments of the invention the SigB sigma factor comprises the amino acid sequence of SEQ ID NO: 94 or a functional homologue thereof having at least 80%, such as at least 85%, such as at least 90% such as at least 95% or 100% sequence identity to SEQ ID NO:94.

Based on the results of the present invention, the theory is that production of heterologous products, in a host cell where the product formation relies on proteins encoded by genes transcribed from a promoter recognized by a sigma factor of said cell, which is active in the cellular growth-phase (housekeeping sigma factors, such as sigma factor D or sigma factor A), will increase in cells lacking or having reduced amounts of the sigma factor of the cell which is active in the stationary-phase of the cell (sigma factor S or sigma factor B) compared to a cell with unchanged ability to generate Sigma factor S or Sigma factor B. Increased production of heterologous products in cells lacking or having reduced amounts of the sigma factor of the cell which is active in the stationary-phase of the cell (Sigma S or Sigma B) may be explained by:
i) increased transcription from promoters recognized by a housekeeping sigma factor. This may benefit transcription of genes essential for normal growth as well as genes directly or indirectly involved in heterologous product formation (if one or more of the genes involved in product formation is transcribed from a Sigma D or Sigma A recognized promoter),
ii) increased expression levels of proteins involved in the protein synthesis machinery. This may result in an increased amount of protein translation, which is beneficial for general growth and/or product formation
iii) energy savings in the cell due to decreased expression of Sigma S transcribed genes.

The increased transcription in i), is particularly useful if the gene(s) for which the increased transcription is achieved is rate limiting in the production of the heterologous product.

Improving transcription of a heterologous nucleic acid involved in the production of a heterologous product may therefore in some instances lead to an indifferent product yield, when the transcription of the particular heterologous nucleic acid is not rate limiting for the process.

Example 1 of the present disclosure shows that deletion of the RpoS gene from E. coli cells producing the heterologous product 2′-FL, remarkedly increased the product yield, whereas deletion of sigma factor H or F did not have any effect on 2′-FL formation. The inventors found that the increase was coupled to a remarkable increase in expression levels of the heterologous nucleic acids incorporated into the genetically engineered cell.

Accordingly, the present invention relates to a genetically engineered cell for the biosynthetic production of one or more heterologous product(s), wherein the function of the sigma factor of the cell which is active in the stationary-phase of the cell, such as RpoS or SigB, is reduced or abolished in said cell. Preferably, the cell is engineered such that the function of the sigma factor of the cell which is active in the stationary-phase of the cell, such as RpoS or SigB is reduced or abolished in said cell. Consequently, it is not intended to cover microorganisms which naturally (e.i., can be found in nature) lack RpoS or SigB, but which have been genetically engineered for other purposes.

As such, the function of RpoS or SigB may be reduced in any number of ways known to the skilled person, such as deletion of the coding gene, or incorporation of mutations into the gene that renders RpoS or SigB less functional, or it may be done by modulation of RpoS or SigB at a transcriptional or translational level, thus reducing the expression level of RpoS or SigB. One way to reduce the transcription level is to exchange the endogenous regulatory elements with elements that results in lower transcription levels, e.g., a weaker promoter, or by changing mRNA stability e.g., by modifying sequences either upstream of rpoS/sigB initiation site or in the start of the gene. The RpoS or SigB gene may in that sense be inactivated by a complete or partial deletion of the corresponding nucleic acid sequence from the bacterial genome, or the gene sequence is mutated in the way that it is not transcribed, or, if transcribed, the transcript is not translated, or if translated to a protein, i.e., modulated in a way so that the protein does not have the corresponding RNA transcription factor activity.

The level of RpoS or SigB may be reduced by any method known to the skilled person, such as but not limited to genomic deletion, silencing mutation, missense mutation, knockdown by small interfering RNA (siRNA) and other methods known to the skilled person.

The term “fully or partially inactivated” in relation to the genes described herein refers to the inactivation of the gene of interest by complete or partial deletion of the corresponding nucleic acid sequence from the genome of the cell.

In embodiments, the gene encoding RpoS or SigB is fully or partially inactivated. In embodiments, the gene encoding RpoS or SigB is knocked down in the genetically engineered cell. In embodiments, the gene encoding RpoS or SigB is knocked out in the genetically engineered cell. In embodiments, the gene encoding RpoS or SigB deleted from the genome of the genetically engineered cell.

The term “knocked down” relates to a non-genomic reduction of expression of the gene of interest, e.g., knock down using a small inhibitory RNA, which inhibits the protein synthesis of the target gene, thus a reduced expression is obtained by non-genomic editing.

The term “knockout” relates to full deletion of the gene or partial deletion of parts of the gene from the genome of the genetically engineered cell, which results in an unfunctional gene product.

### RpoS Regulating Factors

The level of RpoS is regulated by several factors, both transcription factors and translational factors. The rpoS gene is recognized by the RpoD containing RNA polymerase complex, which initiates the expression of the rpoS gene. The transcribed mRNA of the rpoS gene is regulated by a number of additional factors that enhances or reduces the further translation of the mRNA product, such as but not limited to accessory proteins and/or small regulatory RNA molecules.

Accordingly, in embodiments, the function of one or more factors promoting the RpoS function is reduced or abolished. In further embodiments, a gene encoding the one or more RpoS promoting factors is fully or partially inactivated.

Within the present invention “a factor promoting” a function is to be understood as a factor which enhances the functionality compared to the basic function of that gene, DNA, RNA and/or polypeptide. In that regard, a promoting factor may be both DNA, RNA and/or a polypeptide. A promoting factor may exert its function on any biological level. In a non-limiting example, a factor promoting the transcription of a gene, or the translation of RNA to polypeptides, or a factor that promotes the functionality of the functional polypeptide, are all considered promoting factors. In that regard, a “factor” may be a DNA, RNA or polypeptide, and could be both internal factors, such as RNA loop forming or external factors, such as, but not limited to, siRNAs and polypeptides inhibiting the transcription or translation of the RpoS gene.

### RpoS Promoting Factors

In embodiments of the present invention, the function of the sigma factor of the cell which is active in the stationary-phase of the cell is reduced or abolished by fully or partially inactivating or reducing one or more RpoS promoting factors. In further embodiments, the one or more fully or partially inactivated RpoS promoting factors is selected from the group consisting of ArcZ, DksA, GadX, DsrA, DeaD, RprA and Crl.

ArcZ (SEQ ID NO: 9 or a functional homologue thereof) is a conserved small regulatory RNA that increases translation of RpoS. In embodiments, the small regulatory RNA ArcZ is fully or partially inactivated in the genetically engineered cell of the present invention.

DksA (RNA polymerase-binding transcription factor DksA, (SEQ ID NO: 97 or a functional homologue thereof) binds to guanosine tetraphosphate (ppGpp) and destabilizes promoter complexes and decreases the activity of RNAP holoenzymes containing the o factors RpoE, RpoS, and RpoH, but enhances the activity of the RpoD bound RNA polymerase. Thus, upon inactivation of the RpoS gene, it may, in relation to the present invention, be favorable to enhance the DksA level. In embodiments of the present invention, the rpoS gene translational promotion factor, DksA is fully or partially inactivated in a cell comprising a functional rpoS gene. In further embodiments, the gene encoding DksA (SEQ ID NO: 10 or a functional homologue thereof) is fully or partially inactivated in a cell comprising a functional rpoS gene.

HTH-type transcriptional regulator GadX (SEQ ID NO: 98 or a functional homologue thereof) is a positive regulator of rpoS gene transcription and is indispensable upon entry into the stationary phase in response to acidic pH. Accordingly, in embodiments, the rpoS gene transcription promotion factor, GadX is fully or partially inactivated in a genetically engineered cell of the present invention, which comprises a functional rpoS gene. In further embodiments, the gene encoding GadX (SEQ ID NO: 11 or a functional homologue thereof) is fully or partially inactivated in a cell comprising a functional rpoS gene.

The small regulatory RNA DsrA (SEQ ID NO: 12 or a functional homologue thereof) activates the translation of rpoS RNA, and thus activates the production of rpoS. Accordingly, in embodiments, the small regulatory RNA DsrA is fully or partially inactivated fully or partially inactivated in the genetically engineered cell of the present invention.

The ATP-dependent RNA helicase DeaD (SEQ ID NO: 99 or a functional homologue thereof) appears to destabilize mRNA secondary structures in the translation initiation region of mRNAs, and specifically destabilizes the inhibitory stem-loop structures in the 5′ UTR of the rpoS mRNA transcript, thereby enabling annealing of the small regulatory RNA DsrA, which in turn enables translation of rpoS. Thus, in embodiments, the gene encoding the ATP-dependent RNA helicase DeaD (SEQ ID NO: 13 or a functional homologue thereof) is fully or partially inactivated in the genetically engineered cell of the present invention.

In further embodiments, the gene encoding the ATP-dependent RNA helicase DeaD (SEQ ID NO: 13 or a functional homologue thereof) and/or the gene encoding the small regulatory RNA

DsrA (SEQ ID NO: 12 or a functional homologue thereof) are fully or partially inactivated in the genetically engineered cell of the present invention.

The small regulatory RNA RprA (SEQ ID NO: 14 or a functional homologue thereof) is required for production of RpoS in response to osmotic shock. The small regulatory RNA RprA alters secondary structure in the leader sequence of the rpoS mRNA transcripts to facilitate RpoS translation and increase both the accumulation and half-life of rpoS mRNA transcripts. Thus, in embodiments the gene encoding the small regulatory RNA RprA (RprA) is fully or partially inactivated in the genetically engineered cell of the present invention.

Ribonuclease III (RNaselll, SEQ ID NO: 107 or a functional homologue thereof) is a double-stranded RNA-specific endoribonuclease, which binds to or specifically cleaves double-stranded mRNA molecules thereby inducing stability and/or alteration in the molecule conformation. RNase III has been shown to be necessary for the normal increase of the RpoS levels under glucose starvation. In embodiments, RNaselll is fully or partially inactivated in the genetically engineered cell of the present invention. In further embodiments, the gene encoding RNAselll (SEQ ID NO: 106 or a functional homologue thereof) is fully or partially inactivated in the genetically engineered cell of the present invention, preferably a cell comprising a functional rpoS gene.

RNA polymerase holoenzyme assembly factor Crl (Crl, (SEQ ID NO: 92 or a functional homologue thereof) is an RNA polymerase holoenzyme assembly factor which promotes the binding of RpoS to the RNA polymerase and shifts the expression from RpoD driven to RpoS driven expression. Accordingly, in embodiments, the rpoS gene translational promotion factor, Crl is fully or partially inactivated in a genetically engineered cell of the present invention, which comprises a functional rpoS gene. In further embodiments, the gene encoding Crl (SEQ ID NO: 8 or a functional homologue thereof) is fully or partially inactivated in the genetically engineered cell of the present invention. Example 3 of the present invention illustrate that deletion of Crl has a positive effect on 2′FL formation in an RpoS positive cell.

### Factors Inhibiting the RpoS Function

Along the same lines, overexpression of factors inhibiting the RpoS function, either by transcriptionally, translationally reducing the amount of available RpoS or functionally inhibiting the RpoS polypeptide, can reduce the function of the stationary phase RNA polymerase sigma factor in the cell.

In general, an increase in a protein/gene activity may be obtained by overexpression of the gene encoding the protein or by enhancing the gene copy number, selecting a strong promoter or by modulation of the gene transcription or translation factors. Accordingly, in embodiments, the activity of one or more factors inhibiting the RpoS function is/are increased. In further embodiments, the nucleic acid encoding the one or more factors inhibiting the RpoS function is/are overexpressed. In further embodiments, one or more factors inhibiting transcription of the rpoS gene are overexpressed. In further embodiments, one or more factors inhibiting translation of the rpoS gene mRNA transcript are overexpressed. In embodiments, the one or more factors inhibiting the RpoS function is/are selected from the group consisting of RssB, RNase III, H-NS, ArcA, CRP, Fur, MqsA, OxyS and CyaR.

Regulator of RpoS (RssB, SEQ ID NO: 100 or a functional homologue thereof) is an adaptor protein that facilitates degradation of RpoS. Thus, in embodiments the gene encoding the Regulator of RpoS (rssB, SEQ ID NO: 15 or a functional homologue thereof) is overexpressed in the genetically engineered cell of the present invention to increase the levels of RssB with the amino acid sequence of SEQ ID NO: 100 or a functional homologue thereof.

DNA-binding transcriptional regulator H-NS (HNS, SEQ ID NO: 101 or a functional homologue thereof) function as a gene silencer resides in its preferential binding to AT-rich curved DNA sequences often found upstream of E. coli promoters and in its ability to induce bending of noncurved DNA, thus altering DNA conformation/topology and/or competing with RNAP and other regulators. Thus, in embodiments the gene encoding the DNA-binding transcriptional regulator H-NS (hns, SEQ ID NO: 16 or a functional homologue thereof) is overexpressed in the genetically engineered cell of the present invention to increase the levels of HNS with the amino acid sequence of SEQ ID NO: 101 or a functional homologue thereof.

Phosphorylated DNA-binding transcriptional dual regulator ArcA (pArcA, SEQ ID NO: 102 or a functional homologue thereof) acts as a repressor for rpoS transcription. Thus, in embodiments the gene encoding the DNA-binding transcriptional dual regulator ArcA (arcA) is overexpressed in the genetically engineered cell of the present invention.

CAMP receptor protein (CRP, SEQ ID NO: 103 or a functional homologue thereof) regulates the expression of over 180 genes, amongst these cAMP bound CRP inhibits rpoS transcription. Accordingly, in embodiments, the promoter of the present invention is positively regulated by CAMP-bound CRP. In further embodiments the gene encoding the CAMP receptor protein (crp,

SEQ ID NO: 18 or a functional homologue thereof) is overexpressed in the genetically engineered cell of the present invention to increase the levels of CRP with the amino acid sequence of SEQ ID NO: 101 or a functional homologue thereof.

The transcription activator Ferric Uptake Regulation (FUR, SEQ ID NO: 104 or a functional homologue thereof), once Fe²⁺ bound acts as a repressor for rpoS transcription. Thus, in embodiments the gene encoding the Ferric Uptake Regulation (fur, SEQ ID NO: 19 or a functional homologue thereof), is overexpressed in the genetically engineered cell of the present invention to increase the levels of FUR with the amino acid sequence of SEQ ID NO: 101 or a functional homologue thereof.

DNA-binding transcriptional repressor MqsA (MqsA, SEQ ID NO: 105 or a functional homologue thereof) acts as a repressor for rpoS transcription. Thus, in embodiments the gene encoding the DNA-binding transcriptional repressor MqsA (mqsA, SEQ ID NO: 20 or a functional homologue thereof), is overexpressed in the genetically engineered cell of the present invention to increase the levels of MqsA with the amino acid sequence of SEQ ID NO: 105 or a functional homologue thereof.

The small regulatory RNA CyaR (SEQ ID NO: 22 or a functional homologue thereof), is a small RNA that promotes degradation of, e.g., the rpoS gene mRNA transcript in a Hfq dependent manner. Thus, in embodiments the gene encoding the small regulatory RNA CyaR, is overexpressed in the genetically engineered cell of the present invention.

The small regulatory RNA OxyS (SEQ ID NO: 21 or a functional homologue thereof), is involved in the translation of the rpoS gene mRNA transcript and OxyS is expressed in response to oxidative stress and impairs cell division. Thus, in embodiments the gene encoding the small regulatory RNA OxyS, is overexpressed in the genetically engineered cell of the present invention.

### Sigma Factors Active in the Cellular Growth-Phase

Bacterial cells generally express a sigma factor which active in the cellular growth-phase and can therefore be considered as a housekeeping sigma factor. In E. coli the housekeeping sigma factor is sigma factor D (RpoD) and in B. subtilis and other gram-positive bacteria the house keeping sigma factor is sigma factor A (SigA).

RpoD, also known as sigma 70 (σ⁷⁰) is the primary sigma factor during exponential growth, targeting RNA polymerase sigma 70 to a wide range of promoters that are essential for normal growth. Changes away from typical growth conditions, such as heat shock or growth into stationary phase, lead to the replacement of RpoD with other sigma factors in the RNA polymerase holoenzyme complex, such as RpoH or RpoS.

When RpoD binds to a promoter DNA, it contacts both the −10 and −35 regions upstream of the translation start codon simultaneously. In promoters with a −35 site, RpoD is more effective when there is also a proximal half-site or a complete UP element, whereas the stationary phase sigma, RpoS, has opposite selectivity. Both sigma 70 and RNA polymerase sigma factor RpoS compete for available RNA polymerase core complex during stationary phase growth.

In the present application it is predicted that the following motif TT (G/C/T) (A/T) C (A/G) (n)₁₄₋₁₈ TA (T/A) (A/G) (A/T) I represents a RhoD acceptable motif, which is based on the RopD motif TTGACAnnnnnnnnnnnnnnTATAAT disclosed in the review by Schellhorn (Frontiers in Microbiology 2020 Volume 11 Article 560099). The underlined letters in the RhoD acceptable motif correspond to the RhoD motif.

Accordingly, in embodiments of the present invention, promoter sequences comprise an RhoD motif according to the current invention comprise the consensus sequence of TTGACAnnnnnnnnnnnnnnTATAAT, wherein “n” represents any nucleotide. In further embodiments, the promoter sequence comprise an RhoD acceptable motif comprising the consensus sequence of TT (G/C/T) (A/T) C (A/G) (n)₁₄₋₁₈ TA (T/A) (A/G) (A/T) T.

Non-limiting examples of promoters that comprise an RhoD acceptable motif are the promoter of the lac operon, Plac (SEQ ID NO: 44), the promoter of the tagatose-1,6-bisphosphate aldolase 2 (gatY) PgatY (SEQ ID NO: 82) and the promoter of the glycerol facilitator (glpF) PglpF (SEQ ID NO: 35), and the anaerobic glycerol-3-phosphate dehydrogenase subunit A (glpA) PglpA (SEQ ID NO: 83) listed in table 1.

Other promoters, such as, but not limited to the promoter of the D-galactose/methyl-galactoside ABC transporter periplasmic binding protein (mglB), PmgIB (SEQ ID NO: 84), is recognized by both RpoD and RpoS, and may thus be transcribed by either the RpoS or the RpoD bound RNA polymerase.

A reduction in the level of RpoS reduce the competition between RpoD and RpoS in the RNA polymerase holoenzyme complex and therefore enhances the transcription level of RpoD dependent promoters and leads to further overexpression of heterologous nucleic acids comprising RpoD recognized promoter sequences, such as PglpF, Plac and/or PgatY.

Accordingly, in embodiments of the present invention, the mRNA transcript level of the at least one heterologous nucleic acid of the present invention is enhanced when the function of the stationary phase RNA polymerase sigma factor RpoS is reduced or abolished in said cell. In embodiments of the present invention, the mRNA transcript level of the at least one heterologous nucleic acid of the present invention is increased at least 2-fold, such as at least 5-fold, at least 10-fold, at least 20-fold or such as at least 50-fold increased or such as between 2-fold and 10-fold increased, such as between 10-fold and 50-fold increased, such as between 10-fold and 20-fold increased, such as between 25-fold and 50-fold increased, when the function of the stationary phase RNA polymerase sigma factor RpoS is reduced or abolished in said cell.

In further embodiments, the mRNA transcript level of the at least one heterologous nucleic acid of the present invention is increased, when the function of the stationary phase RNA polymerase sigma factor RpoS is reduced or abolished in said cell, and wherein the promoter of the at least one heterologous nucleic acid is selected from the group consisting of Plac, PgatY, PglpF and PglpA, and variants thereof.

In embodiments, the mRNA transcript level of the at least one heterologous nucleic acid encoding a glycosyltransferase is increased at least 2-fold, such as at least 5-fold, at least 10fold, at least 20-fold or such as at least 50-fold increased or such as between 2-fold and 10-fold increased, such as between 10-fold and 50-fold increased, such as between 10-fold and 20-fold increased, such as between 25-fold and 50-fold increased, when the function of the stationary phase RNA polymerase sigma factor RpoS is reduced or abolished in said cell, and when the promoter of the at least one heterologous nucleic acid is selected from the group consisting of Plac, PgatY, PglpF and PglpA, and functional variants thereof.

In embodiments, the mRNA transcript level of the at least one heterologous nucleic acid encoding a glycosyltransferase, one or more CMP-N-acetylneuraminic acid pathway genes, one or more a sucrose utilization gene(s), an MFS transporter, a nucleic acids encoding the heterologous product such as and antibody or enzyme, is increased at least 2-fold, such as at least 5-fold, at least 10-fold, at least 20-fold or such as at least 50-fold increased or such as between 2-fold and 10-fold increased, such as between 10-fold and 50-fold increased, such as between 10-fold and 20-fold increased, such as between 25-fold and 50-fold increased, when the function of the stationary phase RNA polymerase sigma factor RpoS is reduced or abolished in said cell, and when the promoter of the at least one heterologous nucleic acid is selected from the group consisting of Plac, PgatY, PglpF and PglpA, and functional variants thereof.

In embodiments, the mRNA transcript level of at least one recombinant endogenous gene, for which the transcription is regulated by a promoter which is recognized by the RNA polymerase sigma factor RpoD, such as one or more colanic acid genes (gmd, wcaG, manC, manB and/or manA), other relevant endogenous pathway genes and/or lactose permease gene is increased at least 2-fold, such as at least 5-fold, at least 10-fold, at least 20-fold or such as at least 50-fold increased or such as between 2-fold and 10-fold increased, such as between 10-fold and 50-fold increased, such as between 10-fold and 20-fold increased, such as between 25-fold and 50-fold increased, when the function of the stationary phase RNA polymerase sigma factor RpoS is reduced or abolished in said cell, and when the promoter of the at least one recombinant endogenous nucleic acid is selected from the group consisting of Plac, PgatY, PglpF, PglpT and PglpA, and functional variants thereof.

In embodiments, the mRNA transcript level of the at least one heterologous nucleic acid encoding a polypeptide required for the production of the one or more heterologous product(s) is increased when the function of the stationary phase RNA polymerase sigma factor RpoS is reduced or abolished in said cell, and when the promoter of the at least one heterologous nucleic acid is Plac or a functional variant thereof.

In embodiments, the mRNA transcript level of the at least one heterologous nucleic acid encoding a polypeptide required for the production of the one or more heterologous product(s) is increased, when the function of the stationary phase RNA polymerase sigma factor RpoS is reduced or abolished in said cell, and when the promoter of the at least one heterologous nucleic acid is PglpF or a functional variant thereof.

In embodiments, the mRNA transcript level of the at least one heterologous nucleic acid encoding a polypeptide required for the production of the one or more heterologous product(s) is increased, when the function of the stationary phase RNA polymerase sigma factor RpoS is reduced or abolished in said cell, and when the promoter of the at least one heterologous nucleic acid is PglpA or a functional variant thereof.

In embodiments, the mRNA transcript level of the at least one heterologous nucleic acid encoding a polypeptide required for the production of the one or more heterologous product(s) is increased, when the function of the stationary phase RNA polymerase sigma factor RpoS is reduced or abolished in said cell, and when the promoter of the at least one heterologous nucleic acid is PgatY or a functional variant thereof.

In embodiments, the mRNA transcript level of the at least one heterologous nucleic acid encoding a polypeptide required for the production of the one or more heterologous product(s) is increased, when the function of the stationary phase RNA polymerase sigma factor RpoS is reduced or abolished in said cell, and when the promoter of the at least one heterologous nucleic acid is PglpT or a functional variant thereof.

In embodiments, the expression of the at least one heterologous nucleic acid comprises expression from at least one gene selected from the group consisting of one or more glycosyltransferase genes, one or more CMP-N-acetylneuraminic acid pathway genes, one or more transporter genes and one or more nucleic acids encoding the heterologous product to be produced by the genetically engineered cell, and which is driven by an RpoD recognized promoter.

In embodiments, the expression of the at least one heterologous nucleic acid comprises expression from at least one gene selected from the group consisting of one or more glycosyltransferase genes, one or more CMP-N-acetylneuraminic acid pathway genes, one or more transporter genes and one or more nucleic acids encoding the heterologous product to be produced by the genetically engineered cell, is enhanced when the function of the stationary phase RNA polymerase sigma factor RpoS is reduced or abolished in said cell.

In embodiments of the invention the RpoD sigma factor is encoded by a nucleic acid sequence of SEQ ID NO: 2 or a functional homologue thereof having at least 80%, such as at least 85%, such as at least 90% such as at least 95% or 100% sequence identity to SEQ ID NO: 2.

In embodiments of the invention the RpoD sigma factor comprises the amino acid sequence of SEQ ID NO: 91 or a functional homologue thereof having at least 80%, such as at least 85%, such as at least 90% such as at least 95% or 100% sequence identity to SEQ ID NO: 91.

In embodiments of the invention the SigA sigma factor is encoded by a nucleic acid sequence of SEQ ID NO: 95 or a functional homologue thereof having at least 80%, such as at least 85%, such as at least 90% such as at least 95% or 100% sequence identity to SEQ ID NO: 95.

In embodiments of the invention the SigA sigma factor comprises the amino acid sequence of SEQ ID NO: 96 or a functional homologue thereof having at least 80%, such as at least 85%, such as at least 90% such as at least 95% or 100% sequence identity to SEQ ID NO: 96.

### A Heterologous or Recombinant Native Nucleic Acid Sequence

The present invention relates to a genetically engineered cell comprising a heterologous nucleic acid involved in the production of a heterologous product. The heterologous nucleic acid may encode a heterologous product as such and/or a heterologous polypeptide required for the production of the heterologous product.

Preferably, the expression of the heterologous nucleic acid is controlled by a promoter which is recognized by a sigma factor in the cell which is active in the cellular growth-phase, such as the RNA transcription factor RpoD or SigA. As the expression of some RpoD or SigA driven genes is reduced upon entry into the stationary phase, by exchange of RpoD or SigA with RpoS or SigB in the RNA polymerase holoenzyme complex, expression of some nucleic acids under regulation of RpoD or SigA are enhanced upon deletion of RpoS or SigB, respectively. Accordingly, in embodiments, expression of the heterologous nucleic acid is increased when the functionality of RpoS or SigB is reduced. In further embodiments, the heterologous nucleic acid is transcribed in the absence of RpoS or SigB. Preferably, the transcription of the heterologous nucleic acid is increased in the absence of RpoS or SigB.

In the present context, the term “heterologous nucleic acid sequence”, “heterologous gene/nucleic acid/nucleotide sequence/DNA encoding” or “coding nucleic acid sequence” is used interchangeably and refers to a nucleic acid sequence of a different origin than the cell into which it is inserted, e.g. a sequence originating from a different species than the host cell.

A heterologous nucleic acid sequence can be produced in vitro using standard laboratory methods for making nucleic acid sequences and will often be artificial in that it is codon optimized for the host into which it is inserted. The heterologous nucleic acid comprises a set of consecutive, non-overlapping triplets (codons) which is transcribed into mRNA and translated into a protein when under the control of the appropriate control sequences, i.e., a promoter sequence. Preferably, the promoter recognized by RpoD or SigA.

When a promoter is recognized by a specific RNA transcription factor, the transcription of the DNA sequence downstream of the recognized motif, i.e., promoter, is initiated or reduced. In the present invention the indented function obtained by recognition of a promoter by RpoD or

SigA is that transcription of the nucleic acid sequence downstream of the recognized promoter sequence is transcribed.

Furthermore, the present invention also relates to “recombinant nucleic acid sequences” or “recombinant endogenous/native nucleic acid sequence or gene” in the present disclosure meaning a nucleic acid sequence that has been manipulated compared to the native sequence found in the host cell. In that regard a recombinant nucleic acid sequence may be a native/endogenous gene that is inserted into a non-native nucleic acid construct expressed in the cell. A recombinant nucleic acid sequence may also be a native gene expressed from a or non-native chromosomal location in the cell or a native gene wherein the regulatory elements, such as the promoter, upstream of the transcription initiation codon has been exchanged with a different regulatory element, than the regulatory element normally regulating the native gene. An example of a recombinant endogenous nucleic acid can for example be the colonic acid operon of the present invention, wherein the original promoter sequence has been exchanged with another native promoter sequence, such as the PglpF promoter (SEQ ID NO: 35) leading to a specific configuration of the two elements (PglpF promoter and CA operon) not found in the native host cell i.e., they are not naturally operably linked in the host and therefore considered as a recombinant endogenous/native nucleic acid sequence. Heterologous nucleic acids may also be considered as a recombinant nucleic acid since they are by definition non-natural to the host cell.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and heterologous nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The heterologous nucleic acid sequence may be a coding DNA sequence e.g., a gene, or non-coding DNA sequence e.g., a regulatory DNA, such as a promoter sequence or other non-coding regulatory sequences, such as small regulatory RNAs.

The term “nucleic acid construct” means an artificially constructed segment of nucleic acids, in particular a DNA segment, which is intended to be inserted into a host cell, e.g., a bacteria, to modify expression of a gene of the cellular genome, or expression of a gene/coding DNA sequence, which may be included in the construct.

The invention also relates to a heterologous nucleic acid construct comprising a coding nucleic sequence and further comprising a promoter sequence which is recognized by the RNA transcription factor RpoD or SigA, wherein said nucleic acid construct is for expression in a genetically engineered cell wherein the function of the stationary phase RNA polymerase sigma factor RpoS or SigB is reduced or abolished in said cell. Accordingly, a nucleic acid construct of the invention may comprise one or more heterologous DNA sequence encoding a gene of interest, in example one or more glycosyltransferase genes, one or more CMP-N-acetylneuraminic acid pathway genes and one or more transporter genes, one or more nucleic acids encoding the heterologous product to be produced and further comprising one or more non-coding regulatory DNA sequence, e.g., one or more promoter DNA sequences, e.g., a promoter sequence which is recognized by the RNA transcription factor RpoD or SigA, e.g., a promoter derived from the promoter sequence of the lac operon or the glp operon, or a promoter sequence derived from another genomic promoter DNA sequence which is recognized by the RNA transcription factor RpoD, or a synthetic promoter sequence which is recognized by the RNA transcription factor RpoD, and wherein the coding and promoter sequences are operably linked.

A further nucleic acid construct of the invention may comprise a recombinant endogenous gene for which the transcription is regulated by a promoter that is recognized by the RNA transcription factor RpoD or SigA, e.g., a promoter derived from the promoter sequence of the lac operon or the glp operon, or a promoter sequence derived from another genomic promoter DNA sequence which is recognized by the RNA transcription factor RpoD, or a synthetic promoter sequence which is recognized by the RNA transcription factor RpoD, and wherein the coding and promoter sequences are operably linked. In embodiments of the invention recombinant endogenous nucleic acids may be selected from de novo GDP-fucose pathway such as one or more genes from the colanic acid gene cluster (gmd-wcaG—manC-manB-manA), in particular mannose-6-phosphate isomerase (manA) and/or phosphomannomutase (manB), lactose permease, one or genes involved in the biosynthetic pathway for making at one sugar nucleotide selected from the group consisting of glucose-UDP-GIcNAc, UDP-galactose, UDP-glucose, UDP-N-acetylglucosamine, and UDP-N-acetylgalactosamine (GlcNAc).

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

In embodiments, the nucleic acid construct of the invention may be a part of a vector DNA. In other embodiments, the construct it is an expression cassette/cartridge that is integrated in the genome of the genetically engineered cell.

In addition, in embodiments of the present invention, the at least one heterologous nucleic acid involved in the production of the heterologous product is under regulation of a promoter which is recognized by the RNA transcription factor RpoD. In further embodiments the heterologous nucleic acid is under the control of a promoter selected from the group consisting of PglpF,

Plac, PmglB_70UTR, PglpA_70UTR and PglpT_70UTR (SEQ ID NOS: 35, 44, 32, 33, 34, respectively) and variants thereof, preferably the promoter is a strong promoter selected from the group consisting of PglpF and variants thereof (SEQ ID NOs: 30, 35, 36, 37, 38, 40, 41, 42, 45 and 46, respectively).

Preferably, the heterologous nucleic acid sequence or recombinant endogenous nucleic acid sequence is under the control of a promoter sequence selected from promotor sequences with a nucleic acid sequence as identified in Table 1.

TABLE 1
Selected promoter sequences

| Promoter | % Activity | | | Seq ID |
|---|---|---|---|---|
| name | relative to PglpF* | Strength | Reference | in appl. |
| PmglB_70UTR_SD8 | 291% | high | WO2020255054 | 23 |
| PmglB_70UTR_SD10 | 233-281% | high | WO2020255054 | 24 |
| PmglB_54UTR | 197% | high | WO2020255054 | 25 |
| Plac_70UTR | 182-220% | high | WO2019123324 | 26 |
| PmglB_70UTR_SD9 | 180-226% | high | WO2020255054 | 27 |
| PmglB_70UTR_SD4 | 153%-353% | high | WO2020255054 | 28 |
| PmglB_70UTR_SD5 | 146-152% | high | WO2020255054 | 29 |
| PglpF_SD4 | 140-161% | high | WO2019123324 | 30 |
| PmglB_70UTR_SD7 | 127-173% | high | WO2019123324 | 31 |
| PmglB_70UTR | 124-234% | high | WO2020255054 | 32 |
| PgatY_70UTR | 112-162% | high | | 108 |
| PglpA_70UTR | 102-179% | high | WO2019123324 | 33 |
| PglpT_70UTR | 102-240% | high | WO2019123324 | 34 |
| PglpF | 100% | high | WO2019123324 | 35 |
| PglpF_SD10 | 88-96% | high | WO2019123324 | 36 |
| PglpF_SD5 | 82-91% | high | WO2019123324 | 37 |
| PglpF_SD8 | 81-82% | high | WO2019123324 | 38 |
| PmglB_16UTR | 78-171% | high | WO2019123324 | 39 |
| PglpF_SD9 | 73-93% | middle | WO2019123324 | 40 |
| PgatY_54UTR | 52% | | | 109 |
| PglpF_SD7 | 47-57% | middle | WO2019123324 | 41 |
| PglpF_SD6 | 46-47% | middle | WO2019123324 | 42 |
| PglpA_16UTR | 38-64% | middle | WO2019123324 | 43 |
| Plac_wt* | 15-28% | low | WO2019123324 | 44 |
| PgatY_16UTR | 15-25% | | | 110 |
| PglpF_SD3 | 9% | low | WO2019123324 | 45 |
| PglpF_SD1 | 5% | low | WO2019123324 | 46 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically engineered cell or it may be a recombinant promoter, combining heterologous and/or native elements.

One way to increase the production of a heterologous product is to enhance the expression of the heterologous nucleic acid sequence and/or recombinant endogenous nucleic acid sequence involved in the production of the heterologous product such as by increasing the production of a desired enzymatic activity used to produce the heterologous product, such as but not limited to, in the case of human milk oligosaccharides, the glycosyltransferases or enzymes involved in the biosynthetic pathway of precursor synthesis.

Increasing the promoter strength driving the expression of the desired enzyme is one way of doing this. The strength of a promoter can be assed using a lacZ enzyme assay where β-galactosidase activity is assayed as described previously (see e.g. Miller J. H. Experiments in molecular genetics, Cold spring Harbor Laboratory Press, NY, 1972). Briefly, the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

In embodiments, the expression of the heterologous nucleic acid and/or recombinant endogenous nucleic acid sequence of the present invention is under control of a PglpF (SEQ ID NO: 35) or Plac (SEQ ID NO: 44) or PmglB_UTR70 (SEQ ID NO: 32) or PglpA_70UTR (SEQ ID NO: 33) or PglpT_70UTR (SEQ ID NO: 34) promoter or variants thereof such as promoters identified in Table 1. Further suitable variants of PglpF, PglpA_70UTR, PglpT_70UTR and PmglB_70UTR promoter sequences are described in or WO2019/123324 and WO2020/255054 respectively (hereby incorporated by reference).

When using PglpF, PglpA or PglpT promoters, it may be desirable to delete the glpR gene (which codes the DNA-binding transcriptional repressor GlpR) to eliminate the GlpR-imposed repression of transcription from all PglpF, PglpA or PglpT promoters in the cell and in this manner enhances gene expression from all PglpF-PglpA-or PglpT-based cassettes.

When using Plac promoters, it may be desirable to delete the lacl gene (which codes the DNA-binding transcriptional repressor Lacl) to eliminate the Lacl-imposed repression of transcription from all Plac promoters in the cell and in this manner enhances gene expression from all Plac-based cassettes.

When using PgatY promoters, it may be desirable to delete the gatR gene (which codes the DNA-binding transcriptional repressor GatR) to eliminate the GatR-imposed repression of transcription from all PgatY promoters in the cell and in this manner enhances gene expression from all PgatY-based cassettes.

When using PmgIB promoters, it may be desirable to delete the galR or galS gene (which codes the DNA-binding transcriptional repressor GaIR and GalS) to eliminate the GalR-or GalS-imposed repression of transcription from all PmgIB promoters in the cell and in this manner enhances gene expression from all PmgIB-based cassettes.

In embodiments, the expression of the heterologous nucleic acid and/or recombinant endogenous nucleic acid sequence is under control of a regulatory element with medium or low strength can be selected from the group consisting of PglpF_SD9 (SEQ ID NO: 40), PglpF_SD7 (SEQ ID NO: 41), PglpF_SD6 (SEQ ID NO: 42), PglpA_16UTR (SEQ ID NO: 43), PglpF_SD6 (SEQ ID NO: 445), PglpF_SD6 (SEQ ID NO: 46) and Plac (SEQ ID NO: 44).

In embodiments, the expression of the heterologous nucleic acid and/or recombinant endogenous nucleic acid sequence is under control of a regulatory element with high strength can be is selected from the group consisting of Plac_70UTR (SEQ ID NO: 26), PglpF_SD4 (SEQ ID NO: 30), PglpA_70UTR (SEQ ID NO: 33), PglpT_70UTR (SEQ ID NO: 34), PglpF (SEQ ID NO: 35), PglpF_SD10 (SEQ ID NO: 36), PglpF_SD5 (SEQ ID NO: 37) and PglpF_SD8 (SEQ ID NO: 38).

In embodiments, the PmgIB derived promoters in table 1 are only used in a RpoS negative strain.

Integration of the nucleic acid construct of interest comprised in the construct (expression cassette) into the bacterial genome can be achieved by conventional methods, e.g., by using linear cartridges that contain flanking sequences homologous to a specific site on the chromosome, as described for the attTn7-site (Waddell C.S. and Craig N.L., Genes Dev. (1988) Feb;2 (2): 137-49.); methods for genomic integration of nucleic acid sequences in which recombination is mediated by the Red recombinase function of the phage A or the RecE/RecT recombinase function of the Rac prophage (Murphy, J Bacteriol. (1998); 180 (8): 2063-7; Zhang et al., Nature Genetics (1998) 20:123-128 Muyrers et al., EMBO Rep. (2000) 1 (3): 239-243); methods based on Red/ET recombination (Wenzel et al., Chem Biol. (2005), 12 (3): 349-56.; Vetcher et al., Appl Environ Microbiol. (2005);71 (4): 1829-35); or positive clones, i.e., clones that carry the expression cassette, can be selected e.g., by means of a marker gene, or loss or gain of gene function.

In one or more exemplary embodiments, the present disclosure relates to one or more heterologous or native nucleic acid sequences as illustrated in SEQ ID NOs: 1 to 46, 57, 58, 82 to 84, 93, 95, 106, 108 to 111 and 113.

In particular, the present disclosure relates to one or more heterologous or native nucleic acid sequence (a) and/or to functional homologue thereof having a sequence, which is at least 70% identical to SEQ ID NOs: 1 to 46, 57, 58, 82 to 84, 93, 95, 106, 108 to 111 and 113, such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mo/. Biol. 48:443-453) as implemented in the Needle program of the EMBOSS package (EMBOSS: The European Molecular Biology Open Software Suite, Rice et al., 2000, Trends Genet. 16:276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the-nobrief option) is used as the percent identity and is calculated as follows: (Identical Residues x 100)/(Length of Alignment-Total Number of Gaps in Alignment).

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16:276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the-nobrief option) is used as the percent identity and is calculated as follows: (Identical Deoxyribonucleotides×100)/(Length of Alignment-Total Number of Gaps in Alignment).

### Functional Homologue

A functional homologue or functional variant of a protein/nucleic acid sequence as described herein is a protein/nucleic acid sequence with alterations in the genetic code, which retains its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence. In embodiments of the present invention, the functional homologue is at least 80% identical, such as at least 85% identical such as at least 90% identical, such as such as at least 95% identical to the protein/nucleic acid sequence indicated in connection with a give protein, nucleic acid or gene. A functional homologue of any one of the disclosed amino acid or nucleic acid sequences can also have a higher functionality. A functional homologue of any one of the amino acid or nucleic acid sequences shown in table 2, with the exception of the qPCR primer sequences, should ideally be able to participate in the biosynthetic production of the heterologous product in terms of increased yield, export of the product out of the cell or import of substrate for the production, such as import of a precursor to the heterologous product, improved purity/by-product formation, viability of the genetically engineered cell, robustness of the genetically engineered cell according to the disclosure, or reduction in consumables needed for the production.

### Heterologous Products

In the present context, the term “heterologous product” is to be understood as a product which is not endogenously (naturally) produced by the host cell i.e., for a host cell to produce a heterologous product, the host cell needs to be genetically engineered, furthermore a heterologous product is one that is to be separated/purified from the organism it has been produced in. In embodiments, the heterologous product cannot be endogenously produced by the host cell prior to the genetic engineering of the cell. In that regard, a heterologous product is not limited, and may be any product which is produced by the genetically engineered cell, which is not endogenously produced by the cell. As such, canonical L-amino acids, such as L-glutamic acid and L-threonine, are not considered a heterologous product. Natural products produced by the host cell, such as 1-propanol, in line with the above, is also not considered a heterologous product. In one embodiment of the invention the heterologous product is not an alcohol, such as ethanol, 1-propanol, 1-butanol or is not an amino acid such as L-glutamic acid and L-threonine.

In another embodiment of the invention the heterologous product is not an antigen, in particular an antigen for use in vaccination.

In embodiments, the heterologous product is a polypeptide. In further embodiments, the heterologous product is a mammalian polypeptide, such as but not limited to an albumin, a polypeptide hormone, an antibody and fragments thereof and/or an enzyme. In embodiments, the heterologous product is a polypeptide enzyme such as, but not limited to amylases, amidases, proteases, lipases, cellulases, xylanases, mannanases, catalases, pectinases, pullulanases, phytasesprolyl oligopeptidases and lactases (see for example Singh et al 2016 3 Biotech, 6 (2): 174).

In other embodiments, the heterologous product is a product of an enzymatic reaction in said cell. In further embodiments, the heterologous product is a product which is produced by the cell without addition of external substrate to the medium.

In further embodiments, the heterologous product is an oligosaccharide. In further embodiments, the heterologous product is a human milk oligosaccharide.

In further embodiments, the heterologous product is a vitamin, such as but not limited to vitamin A, D, E, K, B1, B2, B3, B5, B6, B9, B12, B7 or vitamin C. In further embodiments, the heterologous product is a cannabinoid. In further embodiments, the heterologous product is a carotenoid, such as but not limited to canthaxanthin, apocarotenal or beta-carotene.

In one embodiment of the invention the heterologous product is selected from the group consisting of heterologous polypeptides and heterologous oligosaccharides, such as HMO's. In embodiments the heterologous product is selected from the group consisting of albumin, a peptide, a polypeptide, an antibody and fragments thereof, an enzyme, an oligosaccharide, a vitamin, a cannabinoid and a carotenoid.

### Oligosaccharides

In presently preferred embodiments, the heterologous product is an oligosaccharide.

In the present context, the term “oligosaccharide” means a sugar polymer containing at least three monosaccharide units, i.e., a tri-, tetra-, penta-, hexa-or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). In embodiments, the heterologous product is one or more oligosaccharide(s).

Preferably, the oligosaccharide is an HMO.

### Human Milk Oligosaccharide (HMO)

Preferred oligosaccharides of the disclosure are human milk oligosaccharides (HMOs). The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (sialyl) moiety. HMO structures are e.g., disclosed by Xi Chen in Chapter 4 of Advances in Carbohydrate Chemistry and Biochemistry 2015 vol 72.

The HMOs have a core structure comprising a lactose unit at the reducing end that can be elongated by one or more β-N-acetyl-lactosaminyl and/or one or more β-lacto-N-biosyl units, and this core structure can be substituted by an α-L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT). In the context of the present invention lactose is not regarded as an HMO species.

In embodiments, the heterologous product is one or more human milk oligosaccharide(s) (HMO(s)).

In embodiments, the heterologous product is one or more, such as 1, 2, 3, 4, 5, 6, 7, 8, 9, or such as 10, human milk oligosaccharide(s) (HMO(s)). In a preferred embodiment the HMO product comprises or consists of 1 to 5 different HMOs, such as 1 HMO, such as 2, 3, 4 or 5 HMOs.

In embodiments, the human milk oligosaccharide(s) is/are selected from lacto-N-triose II (LNT-II) lacto-N-tetraose (LNT), lacto-N-neotetraose (LNnT), lacto-N-neohexaose (LNnH), para-lacto-N-neohexaose (pLNnH), para-lacto-N-hexaose (pLNH), lacto-N-hexaose (LNH), 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In embodiments, the one or more HMOs is one or more fucosylated HMOs, such as but not limited to one or more fucosylated HMOs selected from the group consisting of 2′-FL, 3-FL, DFL, LNFP-I, LNFP-II, LNFP-III, LNFP-V, LNFP-VI, LNDFH-I, LNDFH-II, TF-LNH, F-LNH-I, F-LNH-II, F-LNH-III, DF-LNH-I and DF-LNH-II.

In embodiments, the one or more HMOs is one or more neutral HMO(s), such as but not limited to one or more neutral HMOs selected from the group consisting of LNT-II, LNT, LNnT, LNH and LNnH.

In embodiments, the one or more HMOs is one or more sialylated HMO(s), such as but not limited to one or more sialylated HMOs selected from the group consisting of 3′SL, 6′SL, DSLNT, LST-a, LST-b, LST-c, DS-LNH-I, FDS-LNH-I, FDS-LNH-II and FSL (3′-S-3-FL).

In one embodiment, the genetically modified cell produces one or more fucosylated HMO(s) selected from the group consisting of 2′FL, 3FL, DFL and LNFP-I.

In one embodiment, the genetically modified cell produces one or more sialylated HMO(s) selected from the group consisting of 3′SL, 6′SL, LST-a, and LST-c. In one embodiment, the genetically modified cell produces one or more HMOs selected from the group consisting of 2′FL, 3FL, DFL, 3′SL and 6′SL.

In embodiments, the one or more HMOs comprises one or more complex HMO(s), comprising five or more saccharide units. In further embodiments, heterologous product is a mixture of HMOs, comprising at least one complex HMO and one or more precursors of the complex

HMO. In example a mixture of HMOs may be a mixture comprising LNFP-I, LNFP-II, LNFP-III, LNFP-V and/or LNFP-VI as the complex HMO(s) and LNT-II, LNT and/or LNnT as the precursor HMOs. A mixture of HMOs may further comprise side product HMOs, such as a mixture of HMOs comprising LNFP-I, LNFP-II, LNFP-III, LNFP-V and/or LNFP-VI as the complex HMO(s) and LNT-II, LNT and/or LNnT as the precursor HMOs and/or 2′FL, 3-FL, and/or DFL as the side product HMOs. For the present invention, the term “side product HMO” is to be understood as an HMO, which is directly in the synthesis pathway from lactose to the complex HMO e.g., the synthesis rout for LNFP-I: Lactose is decorated to form LNT-II; LNT-II is decorated to form LNT; and LNT is decorated to form LNFP-I. Thus, in the specific synthesis of LNFP-1, 2′FL can be considered a side product HMO.

In a preferred embodiment, the cell is capable of producing one or more human milk oligosaccharide(s) (HMO(s)).

### Genetic Modifications to Produce One or More Human Milk Oligosaccharide(s) (HMO(s))

Besides the at least one heterologous nucleic acid involved in the production of the heterologous product, the genetically modified cell of the invention may encompass further modifications that enables it to produce one or more human milk oligosaccharide(s).

Above-mentioned modifications relate to metabolic engineering as well as introduction and expression of functional enzymes that enable the production of said one or more HMO(s) from a starting material, that is often lactose.

Thus, the ability of a cell to produce one or more HMO(s) requires multiple genetic modifications. Accordingly, said genetically modified cell of the invention which is capable of producing one or more HMO(s) comprises a) one or more glycosyltransferase gene(s), b) one or more nucleotide-activated sugar pathway gene(s), such as one or more de novo GDP fucose pathway gene(s), one or more CMP-N-acetylneuraminic acid pathway genes, one or more UDP-galactose pathway gene(s) and/or one or more UDP-N-acetylgalactosamine (GlcNAc) pathway gene(s); and c) optionally one or more transporter genes(s).

Accordingly, in embodiments, the genetically engineered cell of the present invention comprises at least one heterologous nucleic acid encoding one or more glycosyltransferases and/or one or more transporter proteins.

As described above, for the cell to be capable of synthesizing one or more HMO(s), the genetically modified cell of the invention must comprise at least one heterologous nucleic acid, in addition to having a reduced or abolished function of the stationary phase RNA polymerase sigma factor RpoS, wherein the heterologous nucleic acid sequence(s) encodes one or more functional enzyme(s) with glycosyltransferase activity. The glycosyltransferase gene may be integrated into the genome (by chromosomal integration) of the genetically modified cell, or alternatively, it may be comprised in a plasmid and expressed as plasmid-borne, as described for the heterologous nucleic acid sequence of the invention. If two or more glycosyltransferases are needed for the genetically modified cell to be capable of producing an HMO two or more heterologous nucleic acids encoding enzymes with different glycosyltransferase activity may be integrated into a nucleic acid construct of the invention or it may be individual nucleic acid sequences, which may be integrated in the genome and/or expressed from a plasmid. For example to produce DFL two glycosyl transferase activities are required when starting from lactose as the initial substrate namely a alpha-1,2-fucosyltransferase (a first heterologous nucleic acid sequence encoding a first glycosyltransferase) in combination with a alpha-1,3-fucosyltranferase (a second heterologous nucleic acid sequence encoding a second glycosyltransferase), where the first and second heterologous nucleic acid sequences can be integrated chromosomally independently from each other or on introduced on separate plasmid or they can be combined into a nucleic acid construct, optionally comprised in the nucleic acid construct of the invention also comprising the features described above.

In one preferred embodiment, both the first and second heterologous nucleic acids encoding one or more glycosyltransferases are stably integrated into the chromosome of the genetically modified cell; in another preferred embodiment the first and second heterologous nucleic acids encoding one or more glycosyltransferases are integrated independently of the heterologous nucleic acid sequence encoding the heterologous polypeptide of the invention. In a further embodiment, the first and second heterologous nucleic acids encoding one or more glycosyltransferases are integrated into the nucleic acid construct of the invention as disclosed. In another embodiment at least one of the heterologous nucleic acid sequence(s) encoding the glycosyltransferase(s) are plasmid-borne.

In embodiments, glycosyltransferase is selected from the group consisting of β-1,3-N-acetyl-glucosaminyltransferase(s), β-1,3-galactosyltransferase(s), β-1,4-galactosyltransferase(s), α-1,2-fucosyltransferase(s), α-1,3-fucosyltransferase, α-2,3-sialyltransferase(s) and α-2,6-sialyltransferase(s).

Beta-1,3-N-acetyl-glucosaminyltransferase

A β-1,3-N-acetyl-glucosaminyltransferase is any protein which comprises the ability of transferring the N-acetyl-glucosamine of UDP-N-acetyl-glucosamine to lactose or another acceptor molecule, in a beta-1,3-linkage. Preferably, a β-1,3-N-acetyl-glucosaminyltransferase used herein does not originate in the species of the genetically engineered cell i.e., the gene encoding the β-1,3-galactosyltransferase is of heterologous origin. Non-limiting examples of β-1,3-N-acetyl-glucosaminyltransferase are given in table 3. β-1,3-N-acetyl-glucosaminyltransferase variants may also be useful, preferably such variants are at least 80%, such as at least 85%, such as at least 90, such as at least 95% identical to one of the β-1,3-N-acetyl-glucosaminyltransferase in table 3.

TABLE 3
List of β-1,3-N-acetyl-glucosaminyltransferase

| Protein | | | |
|---|---|---|---|
| Name | GenBank ID | origin | HMO product |
| lgtA | WP_033911473.1 | Neisseria | LNT-II, LNT, LNnT, LNFP-I, LNFP-II, |
| | WP_002248149 | meningitidis | LNFP-III, LNFP-V, LNFP-VI, LNDFH-I, |
| | AAF42258.1 | | LNDFH-II, LNDFH-III, pLNH, F-pLNH I, |
| | ELK60643.1 | | pLNnH, LST a, LST b, LST c, DSLNT |
| lgtA | AAK70338.1 | Neisseria | LNT-II, LNT, LNnT, LNFP-I, LNFP-II, |
| | ACF31229.1 | gonorrhoeae | LNFP-III, LNFP-V, LNFP-VI, LNDFH-I, |
| | | | LNDFH-II, LNDFH-III, pLNH, F-pLNH I, |
| | | | pLNnH |
| lgtA | AAK02595.1 | Pasteurella | LNT-II, LNT, LNnT, LNFP-I, LNFP-II, |
| | | multocida | LNFP-III, LNFP-V, LNFP-VI, LNDFH-I, |
| | | | LNDFH-II, LNDFH-III, pLNH, F-pLNH I, |
| | | | pLNnH |
| lgtA | EEZ72046.1 | Neisseria | LNT-II, LNT, LNnT, LNFP-I, LNFP-II, |
| | | cinerea | LNFP-III, LNFP-V, LNFP-VI, LNDFH-I, |
| | | | LNDFH-II, LNDFH-III, pLNH, F-pLNH I, |
| | | | pLNnH |
| HD0466 | WP_010944479.1 | Haemophilus | LNT-II, LNT, LNnT, LNFP-I, LNFP-II, |
| | | ducreyi | LNFP-III, LNFP-V, LNFP-VI, LNDFH-I, |
| | | | LNDFH-II, LNDFH-III, pLNH, F-pLNH I, |
| | | | pLNnH, LST a, LST b, LST c, DSLNT |
| PmnagT | WP_014390683.1 | Pasteurella | LNT-II, LNT, LNnT, LNFP-I, LNFP-II, |
| | | multocida | LNFP-III, LNFP-V, LNFP-VI, LNDFH-I, |
| | | | LNDFH-II, LNDFH-III, pLNH, F-pLNH I, |
| | | | pLNnH, LST a, LST b, LST c, DSLNT |

### β-1,6-N-acetylglucosaminyltransferase

A β-1,6-N-acetyl-glucosaminyl-transferase is any protein which comprises the ability of transferring the N-acetyl-glucosamine of UDP-N-acetyl-glucosamine to an acceptor molecule, in a beta-1,6-linkage. Preferably, a β-1,6-N-acetyl-glucosaminyl-transferase used herein does not originate in the species of the genetically engineered cell i.e., the gene encoding the β-1,6-galactosyltransferase is of heterologous origin. In the context of the resent invention the acceptor molecule, is an acceptor oligosaccharide of at least three or four monosaccharide units, e.g., LNT or LNnT, or more complex HMO structures Some of the examples below use the heterologous β-1,6-N-acetyl-glucosaminyl-transferase named Csp2 from Chryseobacterium sp. KBW03 with GenBank ID NO WP_22844786.1, or a variant thereof to produce for example LNH or LNnH.

β-1,3-galactosyltransferase

A β-1,3-Galactosyltransferase is any protein that comprises the ability of transferring the galactose of UDP-Galactose to a N-acetyl-glucosaminyl moiety to an acceptor molecule in a beta-1,3-linkage. Preferably, a 3-1,3-galactosyltransferase used herein does not originate in the species of the genetically engineered cell i.e., the gene encoding the β-1,3-galactosyltransferase is of heterologous origin. Non-limiting examples of β-1,3-galactosyltransferases are given in table 4. 3-1,3-galactosyltransferases variants may also be useful, preferably such variants are at least 80%, such as at least 85%, such as at least 90, such as at least 95% identical to one of the β-1,3-galactosyltransferases in table 4.

TABLE 4
List of beta-1,3-glycosyltransferases

| Protein | | | HMO |
|---|---|---|---|
| Name | GenBank ID | Origin | product |
| WbgO | WP_000582563.1 | E. coli 055:H7 | LNT |
| cpslBJ | AB050723.1 | Streptococcus | LNnT |
| | | agalactiae | |
| | | type Ib OI2 | |
| Jhp0563 | AEZ55696.1 | Helicobacter | LNnT |
| | | pylori | |
| GalTK | WP_111735921.1 | Helicobacter | LNT |
| | | pylori | |
| | | ATCC 43504 | |
| CvB3GalT | WP_080969100.1 | Chromobacterium | LNT |
| | | violaceum | |

β-1,4-galactosyltransferase

A β-1,4-Galactosyltransferase is any protein that comprises the ability of transferring the galactose of UDP-Galactose to a N-acetyl-glucosaminyl moiety. Preferably, a β-1,4-galactosyltransferase used herein does not originate in the species of the genetically engineered cell i.e., the gene encoding the β-1,4-galactosyltransferase is of heterologous origin. Non-limiting examples of β-1,4-galactosyltransferases are given in table 5. β-1,4-galactosyltransferases variants may also be useful, preferably such variants are at least 80%, such as at least 85%, such as at least 90, such as at least 95% identical to one of the β-1,4-galactosyltransferases in table 5.

TABLE 5
List of beta-1,4-glycosyltransferases

| Protein | | | HMO |
|---|---|---|---|
| Name | GenBank ID | Origin | product |
| GalT | WP_001262061.1 | Helicobacter | LNnT |
| | | pylori | |
| LgtB | AAF42257.1 | Neisseria | LNnT |
| | | meningitidis | |
| | | MC58 | |

Alpha-1,2-fucosyltransferase

An α-1,2-fucosyltransferase is a protein that comprises the ability to catalyze the transfer of fucose from a donor substrate, for example, GDP-fucose, to an acceptor molecule in an alpha-1,2-linkage. Preferably, an alpha-1, 2-fucosyltransferase used herein does not originate in the species of the genetically engineered cell i.e., the gene encoding the alpha-1,2-fucosyltransferase is of heterologous origin. Non-limiting examples of alpha-1,2-fucosyltransferase are given in table 6. Alpha-1,2-fucosyltransferase variants may also be useful, preferably such variants are at least 80%, such as at least 85%, such as at least 90, such as at least 95% identical to one of the alpha-1,2-fucosyltransferase in table 6.

TABLE 6
List of α-1,2-fucosyltransferase

| Protein | | | HMO |
|---|---|---|---|
| Name | GenBank ID | Origin | product |
| FutC | WP_080473865.1 | Helicobacter | 2′FL, |
| | | pylori | DFL, |
| | | ATCC 26695 | LNFP-I |
| wbgL | ADN43847 | E. coli O126 | DFL |
| Smob | WP_126455392.1 | Sulfuriflexus | LNFP-I |
| | | mobilis | |
| FucT2_HpUA802 | AAC99764.1 | Helicobacter | 2′FL, |
| | | pylori | LNFP-I, |
| | | | LNDFH-I |
| FucT2_EcO126t | ABE98421.1 | Escherichia coli | 2′FL, |
| | | | LNFP-I, |
| | | | LNDFH-I |
| FucT2_Hm12198 | CBG40460.1 | Helicobacter | 2′FL, |
| | | mustelae | LNFP-I, |
| | | 12198 | LNDFH-I |
| FucT2_Pm9515 | ABM71599.1 | Brochlorococcus | 2′FL, |
| | | marinus | LNFP-I, |
| | | str. MIT 9515 | LNDFH-I |
| FucT2_HpF57 | BAJ59215.1 | Helicobacter | 2′FL, |
| | | pylori F57 | LNDFH-I, |
| | | | LNDFH-I |
| FucT54 | ADE13114.1 | Sideroxydans | 2′FL, |
| | | lithotrophicus | LNFP-I, |
| | | ES-11 | LNDFH-I |
| Mtun | WP_031437198.1 | Methylobacter | 2′FL, |
| | | tundripaludum | LNFP-I, |
| | | | LNDFH-I |

Alpha-1,3-fucosyltranferase

An alpha-1,3-fucosyltranferase refer to a glycosyltransferase that catalyzes the transfer of fucose from a donor substrate for example, GDP-fucose, to an acceptor molecule in an alpha-1,3-linkage. Preferably, an alpha-1,3-fucosyltransferase used herein does not originate in the species of the genetically engineered cell i.e., the gene encoding the alpha-1,3-fucosyltransferase is of heterologous origin. Non-limiting examples of alpha-1,3-fucosyltransferase are given in table 7. Alpha-1,3-fucosyltransferase variants may also be useful, preferably such variants are at least 80%, such as at least 85%, such as at least 90, such as at least 95% identical to one of the alpha-1,3-fucosyltransferase in table 7.

TABLE 7
List of α-1,3-fucosyltransferase

| Protein | | | HMO |
|---|---|---|---|
| Name | GenBank ID | Origin | product |
| FucT | AAB81031.1 | Helicobacter | 3FL, DFL, |
| | | pylori | LNFP-III, |
| | | NCTC 11639 | LNDFH-III |
| FutA | WP_000487428.1 | Helicobacter | 3FL, DFL, |
| | | pylori | LNFP-VI, |
| | | ATCC 26695 | LNDFH-III |
| CafC | WP_007483358.1 | Bacteroides | 3FL, |
| | | nordii | LNDFH-III |
| FucT6_3_Bf | CAH09151.1 | Bacteroides | 3FL, LNFP-V, |
| | | fragilis | LNDFH-II |
| | | NCTC 9343 | |
| FucT7_3_Bf | CAH09495.1 | Bacteroides | 3FL, LNFP-V, |
| | | fragilis | LNDFH-II |
| | | NCTC 9343 | |
| FucT_3_Am | ACD04596.1 | Akkermansia | 3FL, LNFP-V, |
| | | muciniphila | LNDFH-II |
| | | ATCC BAA-835 | |
| MAMA_R764 | AGC02224.1 | Acanthamoeba | 3FL, LNFP-V, |
| | | polyphaga | LNDFH-II |
| | | moumouvirus | |
| Mg791 | AEQ33441.1 | Megavirus | 3FL, LNFP-V, |
| | | chiliensis | LNDFH-II |
| Moumou_00703 | YP_007354660.1 | Acanthamoeba | 3FL, LNFP-V, |
| | | polyphaga | LNDFH-II |
| | | moumouvirus | |

Alpha-1,3/4-fucosyltransferase

An alpha-1,3/4-fucosyltransferase refer to a glycosyltransferase that catalyzes the transfer of fucose from a donor substrate for example, GDP-fucose, to an acceptor molecule in an alpha-1,3-or alpha 1,4-linkage. Preferably, an alpha-1,3/4-fucosyltransferase used herein does not originate in the species of the genetically engineered cell i.e., the gene encoding the alpha-1,3/4-fucosyltransferase is of heterologous origin. Non-limiting examples of alpha-1,3/4-fucosyltransferase are given in table 8. alpha-1,3/4-fucosyltransferase variants may also be useful, preferably such variants are at least 80%, such as at least 85%, such as at least 90, such as at least 95% identical to one of the alpha-1,3/4-fucosyltransferase in table 8.

TABLE 8
List of α-1,3/4-fucosyltransferase

| Protein | | | HMO |
|---|---|---|---|
| Name | GenBank ID | Origin | product |
| FucTill | AY450598.1 | Helicobacter | LNFP-II, |
| | | pylori | LNFP-V, |
| | | strain DSM 6709 | LNDFH-I, |
| | | | LNDFH-II, |
| FutA | WP_000487428.1 | Helicobacter | DFL, LNFP-V, |
| | | pylori | LNDFH-II |
| | | ATCC 26695 | |

Alpha-2,3-sialyltransferase

An α-2,3-sialyltransferase refer to a glycosyltransferase that catalyzes the transfer of sialyl from a donor substrate for example, CMP-N-acetylneuraminic acid, to an acceptor molecule in an alpha-2,3-linkage. Preferably, an alpha-2,3-sialyltransferase used herein does not originate in the species of the genetically engineered cell i.e., the gene encoding the 2,3-sialyltransferase is of heterologous origin. Non-limiting examples α-2,3-sialyltransferase are given in table 9. α-2,3-sialyltransferase variants may also be useful, preferably such variants are at least 80%, such as at least 85%, such as at least 90, such as at least 95% identical to one of the α-2,3-sialyltransferase in table 9.

TABLE 9
List of α-2,3-sialyltransferase

| Protein | | | HMO |
|---|---|---|---|
| Name | GenBank ID | Origin | product |
| Avi | WP_115249238.1 | Avibacterium | 3′-SL |
| | | avium | |
| Ccol | WP_075498955.1 | Campylobacter | 3′-SL, |
| | | coli | FSL |
| Ccol2 | EAH6554614.1 | Campylobacter | 3′-SL, |
| | | coli | 6′SL, LST-a |
| Chepa | WP_066776435.1 | Campylobacter | 3′-SL, |
| | | hepaticus | 6′SL, LST-a |
| Cinf1 | WP_011272254.1 | Haemophilus | 3′-SL, |
| | | influenzae | 6′SL |
| Cjej1 | EBD1936710.1 | Campylobacter | 3′-SL, |
| | | jejuni | 6′SL, LST-a |
| Clari1 | EGK8106227.1 | Campylobacter | 3′-SL, |
| | | lari | LST-a |
| Cstl | AAF13495.1 | Campylobacter | 3′-SL, |
| | | jejuni | LST-a |
| CstII | AAF31771.1 | Campylobacter | 3′-SL, |
| | | jejuni | 6′SL, LST-a |
| Csub1 | WP_039664428.1 | Campylobacter | 3′-SL |
| | | subantarcticus | |
| MhnNBse | WP_176810284.1 | Mannheimia | 3′-SL |
| | | (multispecies) | |
| Neigon | AAW89748.1 | Neisseria | 3′-SL |
| | | gonorrhoeae | |
| | | FA 1090 | |
| Nst | AAC44541.1 | Neisseria | 3′-SL |
| | | meningitidis | |
| | | MC58 | |
| PM70 | AAK03258.1 | Pasteurella | 3′-SL |
| | | multocida | |
| | | subsp. multocida | |
| | | str. Pm70 | |
| PmN | WP_005726268.1 | Pasteurella | 3′-SL |
| | | (multispecies) | |
| Pmult | WP_005753497.1 | Pasteurela | 3′-SL |
| | | multocida | |
| Poral | WP_101774487.1 | Pasteurella | 3′-SL |
| | | oralis | |
| Poral_2 | WP_101774701.1 | Pasteurella | 3′-SL, |
| | | oralis | 6′SL |

Alpha-2,6-sialyltransferase

An alpha-2,6-sialyltransferase refer to a glycosyltransferase that catalyzes the transfer of sialyl 5 from a donor substrate for example, CMP-N-acetylneuraminic acid, to an acceptor molecule in an alpha-2,6-linkage. Preferably, an alpha-2,6-sialyltransferase used herein does not originate in the species of the genetically engineered cell i.e., the gene encoding the 2,6-sialyltransferase is of heterologous origin. Non-limiting examples α-2,6-sialyltransferase are given in table 10. α-2,6-sialyltransferase variants may also be useful, preferably such variants are at least 80%, such as at least 85%, such as at least 90, such as at least 95% identical to 10 one of the α-2,6-sialyltransferase in table 10.

TABLE 10
List of α-2, 6-sialyltransferase

| Protein | | | HMO |
|---|---|---|---|
| Name | GenBank ID | Origin | product |
| HAC1268 | STP03859.1 | Helicobacter | LST-c |
| | | acinonychis | |
| | | str. Sheeba | |
| Pts6-119 | AB500947.1 | Photobacterium | 6′SL, |
| | | leiognathi | LST-b |
| | | JT-SHIZ-119 | |
| Pts6-224 | BAF92026.1 | Photobacterium | 6, SL, |
| | | sp JT-ISH-224 | LST-b, |
| | | | DSLNT |
| PIST6_145 | BAF91416.1 | Photobacterium | 6′SL, |
| | | leiognathi | LST-b |

Pathways to produce nucleotide-activated sugars

When carrying out the method of this invention, a glycosyltransferase mediated glycosylation reaction preferably takes place in which an activated sugar nucleotide serves as donor. An activated sugar nucleotide generally has a phosphorylated glycosyl residue attached to a nucleoside, a specific glycosyl transferase enzyme accepts only a specific sugar nucleotide. Thus, preferably the following activated sugar nucleotides are involved in the glycosyl transfer: glucose-UDP-GIcNAc, GDP-fucose, UDP-galactose, UDP-glucose, UDP-N-acetylglucosamine, UDP-N-acetylgalactosamine (GlcNAc) and CMP-N-acetylneuraminic acid. The genetically modified cell according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In table 11 below are non-limiting examples of glycosyl-doners and the HMO products they can be used to produce, the list may not be exhaustive.

TABLE 11
glycosyl-donor HMO product list

| Glycosyl- | |
|---|---|
| donor | HMO product |
| UDP-GIcNAc | LNT, LNnT, LNFP-I, LNFP-II, LNFP-III, LNFP-V, LNFP-VI, |
| | LNDFH-I, LNDFH-II, LNDFH-III, LNH, LNnH, pLNH, pLNnH, |
| | F-pLNH-I, F-pLNH-II, F-pLNH-I, F-pLNnH-II, FLSTa, |
| | FLSTb, FLSTc, FLSTd, LSTa, LSTb, LSTc, LSTd, DSLNT, |
| | SLNH, SLNH-II |
| UDP-Gal | LNT, LNnT, LNFP-I, LNFP-II, LNFP-III, LNFP-V, LNFP-VI, |
| | LNDFH-I, LNDFH-II, LNDFH-III, LNH, LNnH, pLNH, pLNnH, |
| | F-pLNH-I, F-pLNH-II, F-pLNH-I, F-pLNnH-II, FLSTa, FLSTb, |
| | FLSTc, FLSTd, LSTa, LSTb, LSTc, LSTd, DSLNT, SLNH-I, |
| | SLNH-II |
| GDP-fucose | 2′FL, 3FL, DFL, LNFP-I, LNFP-II, LNFP-III, LNFP-V, |
| | LNFP-VI, LNDFH-I, LNDFH-II, LNDFH-III, F-LNH, F-LNnH, |
| | F-pLNH-I, F-pLNH-II, F-pLNH-I, F-pLNnH-II, FSL, FLSTa, |
| | FLSTb, FLSTc, FLSTd |
| CMP-Neu5Ac | 3′SL, 6′SL, FSL, FLSTa, FLSTb, FLSTc, FLSTd, LSTa, |
| | LSTb, LSTc, LSTd, DSLNT, SLNH-I, SLNH-II |

In one embodiment of the method, the genetically engineered cell is capable of producing one or more activated sugar nucleotide mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)). The enzymes involved in the de novo biosynthetic pathway of an activated sugar nucleotide can be naturally present in the cell or introduced into the cell by means of gene technology or recombinant DNA techniques, all of them are parts of the general knowledge of the skilled person. In embodiments, the expression of one or more of the enzymes involved in activated sugar nucleotide synthesis is/are regulated by a promoter according to the present invention.

In another embodiment, the genetically modified cell can utilize salvaged monosaccharide for producing activated sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the cell used for genetic modification.

Sialic acid catabolic and biosynthetic pathways

In some embodiments, the genetically modified cell, contains a deficient sialic acid catabolic pathway. By “sialic acid catabolic pathway” is meant a sequence of reactions, usually controlled and catalyzed by enzymes, which results in the degradation of sialic acid. An exemplary sialic acid catabolic pathway described herein is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A deficient sialic acid catabolic pathway is rendered in the E. coli host by introducing a mutation in the endogenous nanA (N-acetylneuraminate lyase) (e.g., GenBank Accession Number D00067.1 (GL216588)) and/or nanK (N-acetylmannosamine kinase) (e.g., GenBank Accession Number (amino acid) BAE77265.1 (GL85676015)), and/or nanE (N-acetylmannosamine-6-phosphate epimerase, GI: 947745, incorporated herein by reference). Optionally, the nanT (N-acetylneuraminate transporter) gene is also inactivated or mutated. Other intermediates of sialic acid metabolism include: (ManNAc-6-P)N-acetylmannosamine-6-phosphate; (GlcNAc-6-P)N-acetylglucosamine-6-phosphate; (GlcN-6-P) Glucosamine-6-phosphate, and (Fruc-6-P) Fructose-6-phosphate. In some preferred embodiments, nanA is mutated. In other preferred embodiments, nanA and nanK are mutated, while nanE remains functional. In another preferred embodiment, nanA and nanE are mutated, while nanK has not been mutated, inactivated or deleted. A mutation is one or more changes in the nucleic acid sequence coding the gene product of nanA, nank, nanE, and/or nanT. For example, the mutation may be 1, 2, up to 5, up to 10, up to 25, up to 50 or up to 100 changes in the nucleic acid sequence. For example, the nanA, nank, nanE, and/or nanT genes are mutated by a null mutation. Null mutations as described herein encompass amino acid substitutions, additions, deletions, or insertions, which either cause a loss of function of the enzyme (i.e., reduced or no activity) or loss of the enzyme (i.e., no gene product). By “deleted” is meant that the coding region is removed completely or in part such that no (functional) gene product is produced. By inactivated is meant that the polypeptide coding nucleic acid sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. A “not mutated” gene or protein does not differ from a native, naturally occurring, or endogenous coding sequence by 1, 2, up to 5, up to 10, up to 20, up to 50, up to 100, up to 200 or up to 500 or more codons, or to the corresponding encoded amino acid sequence.

Furthermore, the bacterium (e.g., E. coli) may also comprise a sialic acid synthetic capability. For example, the bacterium comprises a sialic acid synthetic capability through provision of an exogenous UDP-GIcNAc 2-epimerase (e.g., neuC of Campylobacter jejuni (GenBank AAK91727.1) or equivalent (e.g. (GenBank CAR04561.1)), a Neu5Ac synthase (e.g., neuB of C. jejuni (GenBank AAK91726.1) or equivalent, (e.g. Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g. Vibrio brasiliensis CMP-sialic acid synthase, GenBank WP_006881452.1).

### Colanic Acid Gene Cluster

For the production of fucosylated HMOs the de novo GDP-fucose pathway is important to ensure presence of sufficient GDP-fucose. The colanic acid gene cluster of Escherichia coli is responsible for the production of the extracellular polysaccharide colanic acid, a major oligosaccharide of the bacterial cell wall.

For the production of GDP-fucose four of the colanic acid gene cluster genes encodes the enzymes involved in the de novo synthesis of GDP-fucose (gmd, wcaG, manB, manC), whereas one or several of the genes downstream of GDP-L-fucose, such as wcaJ, can be deleted to prevent conversion of GDP-fucose to colanic acid. wcaH and wcal are also part of the colonic acid gene cluster (operon), but not needed for the production of GDP-fucose. However, a further enzyme, ManA, is part of the de novo GDP-fucose pathway and is encoded from a gene independent of the colonic acid gene cluster.

To secure sufficient amounts of GDP-fucose the promoter of the native colanic acid gene cluster may be exchanged with a stronger promoter, generating a recombinant colanic acid gene cluster, to drive additional production of GDP-fucose. Furthermore, an extra copy of the colanic acid gene cluster can be introduced in the genetically engineered cells as described in the examples. In relation to the present invention the colanic acid gene cluster(s) are preferably regulated by a promoter which is recognized by the a sigma factor of the cell which active in the cellular growth-phase, such as RpoD or SigA. The promoter may on selected from table 1, in particular a PglpF derived promoter.

In embodiments, the colanic acid gene cluster may be expressed from its native genomic locus. The expression may be actively modulated. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC: 2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr POAC88), which catalyzes the conversion of GDP-mannose to GDP-4-dehydro-6-deoxy-D-mannose;
v) wcaG (fcl) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster and/or one or more genes of the de novo GDP-fucose pathway selected from the group consisting of manA, manB, manC, gmd and wcaG.

In further embodiments, the overexpression of one or more of the genes of the de novo GDP-fucose pathway is obtained by addition of introduction of additional copies of the one or more genes encoding either the entire colonic acid gene cluster or one or more genes of the colanic acid gene cluster and/or ManA, preferably with a promoter which is recognized by a sigma factor of the cell which active in the cellular growth-phase, such as RpoD or SigA upstream of one or more additional colanic acid gene cluster genes.

In embodiments, the genetically engineered produces a fucosylated oligosaccharide and mannose-6-phosphate isomerase (manA) and/or phosphomannomutase (manB) is overexpressed.

In embodiments, the oligosaccharide is a fucosylated oligosaccharide and an additional copy of manA encoding mannose-6-phosphate isomerase is inserted into the genome of the genetically modified cell. Preferably with a promoter which is recognized by a sigma factor of the cell which active in the cellular growth-phase, such as RpoD or SigA.

In embodiments, the oligosaccharide is a fucosylated oligosaccharide and an additional copy of manB encoding phosphomannomutase is inserted into the genome of the genetically modified cell. Preferably with a promoter which is recognized by a sigma factor of the cell which active in the cellular growth-phase, such as RpoD or SigA.

In embodiments, the oligosaccharide is a fucosylated oligosaccharide and an additional copy of both manA and manB are inserted into the genome of the genetically modified cell. Preferably with a promoter which is recognized by a sigma factor of the cell which active in the cellular growth-phase, such as RpoD or SigA.

In embodiments, the genetically engineered cell of the present invention comprises at least one additional copy of manB compared to the number of copies of manC (encoding mannose-1-phosphate guanyltransferase) and gmd (encoding GDP-D-mannose-4, 6-dehydratase) and wcaG (encoding GDP-L-fucose synthase).

In embodiments, the genetically engineered cell of the present invention comprises at least the same or more copies of manA compared to the number of copies of manC (encoding mannose-1-phosphate guanyltransferase) and gmd (encoding GDP-D-mannose-4, 6-dehydratase) and wcaG (encoding GDP-L-fucose synthase).

In embodiments, the genetically engineered cell of the present invention comprises at least two copies of manA and three copies of manB. In the context of the present invention, when assessing the number of copies of a gene, the native endogenous copy is included in the count. Hence, three copies of manB can be achieved from the native colonic acid cluster and the insertion of an additional colonic acid cluster (SEQ ID NO: 57) and the insertion of a manB encoding sequence independent of the colonic acid cluster.

### Lactose Permease

Lactose permease is a membrane protein which is a member of the major facilitator superfamily and can be classified as a symporter, which uses the proton gradient towards the cell to transport β-galactosides such as lactose in the same direction into the cell. In oligosaccharide, especially in production of human milk oligosaccharides (HMOs), lactose is often the initial substrate being decorated to produce any HMO of interest a bioconversion that happens in the cell interior. Thus, in production of HMOs, there is a desire to be able to import lactose into the cell, e.g., by expression of a lactose permease such as lacY of E. coli K.-12. In embodiments, the nucleic acid encoding a lactose permease is under control of a promoter which is recognized by the RNA transcription factor RpoD according to the present invention.

In embodiments, the lactose permease is as shown in SEQ ID NO: 85, or a functional homologue thereof having an amino acid sequence which is at least 80% identical, such as at least 85%, 90% or 95% identical to SEQ ID NO: 85.

In further embodiments, the lactose permease is LacY, and the nucleic acid sequence encoding LacY is under control of a promoter which is recognized by a sigma factor of the cell which active in the cellular growth-phase, such as RpoD or SigA.

In embodiments, the expression of the lactose permease is regulated by a promoter according to the present invention.

β-galactosidase

A host cell suitable for HMO production, e.g., E. coli, may comprise an endogenous β-galactosidase gene or an exogenous β-galactosidase gene, e.g., E. coli comprises an endogenous lacZ gene (e.g., GenBank Accession Number V00296 (GI: 41901)). For the purposes of the invention, when producing an HMO, the genetically engineered cell is genetically manipulated to either not comprise any β-galactosidase gene or to comprise a β-galactosidase gene that is inactivated. The gene may be inactivated by a complete or partial deletion of the corresponding nucleic acid sequence from the bacterial genome, or the gene sequence is mutated in the way that it is not transcribed, or, if transcribed, the transcript is not translated or if translated to a protein (i.e., β-galactosidase), the protein does not have the corresponding enzymatic activity. In this way the HMO-producing bacterium accumulates an increased intracellular lactose pool which is beneficial for the production of HMOs.

### Major Facilitator Superfamily (MFS) Transporter Proteins

The heterologous product, such as an HMO, can be accumulated both in the intra- and the extracellular matrix. The product can be transported to the supernatant in a passive way, i.e., it diffuses outside across the cell membrane. The more complex heterologous products may remain in the cell, which is likely to eventually impair cellular growth, thereby affecting the possible total yield of the product from a single fermentation. The product transport, and especially HMO transport, can be facilitated by major facilitator superfamily transporter proteins that promote the effluence of sugar derivatives from the cell to the supernatant. The major facilitator superfamily transporter can be heterologous or native and can be overexpressed under the conditions of the fermentation to enhance the export of the products or derivates thereof such as HMOs. The specificity towards the specific heterologous product, such as an

HMO with a specific sugar moiety can be altered by mutation by means of known recombinant DNA techniques.

Thus, the genetically engineered cell according to the present invention can further comprise a nucleic acid sequence encoding a major facilitator superfamily transporter protein capable of exporting the heterologous product or products.

In the recent years, several new and efficient major facilitator superfamily transporter proteins have been identified, each having specificity for different recombinantly produced HMOs and development of recombinant cells expressing said proteins are advantageous for high scale industrial HMO manufacturing.

Thus, in one or more exemplary embodiments, the genetically engineered cell according to the method described herein further comprises a heterologous nucleic acid encoding a transport protein that acts as a major facilitator superfamily transporter. The gene product that acts as a major facilitator superfamily transporter may be encoded by a heterologous nucleic acid sequence that is expressed in the genetically engineered cell. The heterologous nucleic acid sequence encoding a major facilitator superfamily transporter, may be integrated into the genome of the genetically engineered cell, or expressed using a plasmid.

In one embodiment, the genetically engineered cell of the invention comprises a nucleic acid sequence encoding a major facilitator superfamily transporter protein capable of exporting the heterologous product.

Accordingly, in embodiments, the genetically engineered cell expresses at least one heterologous MFS sugar transporter. In further embodiments the the heterologous MFS sugar transporter is selected from the group consisting of Nec, YberC, Fred, Bad and Vag.

The genetically engineered cell of the present disclosure thus in embodiments expresses a heterologous MFS transporter protein selected from the group consisting of Vag, Nec, Fred, Marc, YberC, Bad and a functional homologue of any one of Vag, Nec, Fred, Marc, YberC or Bad having an amino acid sequence which is at least 70%, such as at least 75%, 80%, 85%, 90%, 95% or 99% identical to the amino acid sequence of any one of SEQ ID NOs: 89, 88, 52, 86, 51 or 87.

Preferably, the MFS transporter protein is selected from Nec (SEQ ID NO: 51) or Marc (SEQ ID NO: 52) and functional homologues thereof having an amino acid sequence which is at least 70%, such as at least 75%, 80%, 85%, 90%, 95% or 99% identical to the amino acid sequence of SEQ ID NO: 51 or 52.

### Nec

In the current context, said MFS transporter protein is preferably a heterologous gene encoding a putative MFS (major facilitator superfamily) transporter protein, originating from the bacterium Rosenbergiella nectarea, identified herein as Nec. Nec has the amino acid sequence of SEQ ID NO: 51; The amino acid sequence identified herein as SEQ ID NO: 51 is an amino acid sequence that has 100% identity with the amino acid sequence having the GenBank accession ID WP_092672081.1.

In one or more exemplary embodiment(s), the MFS transporter, expressed according to the present disclosure is Nec. The genetically engineered cell of the present disclosure thus in one or more exemplary embodiment(s) expresses a heterologous MFS transporter having at least 80%, such as at least 85%, such as at least 90% such as at least 95% or 100% sequence identity to the amino acid sequence of SEQ ID NO: 51.

In further embodiments, the nucleic acid sequence encoding Nec is under control of a promoter which is recognized by a sigma factor of the cell which is active in the cellular growth-phase, such as RpoD or SigA.

### Bad

The MFS transporter protein identified herein as “Bad protein” or “Bad transporter” or “Bad”, interchangeably, has the amino acid sequence of SEQ ID NO: 86; The amino acid sequence identified herein as SEQ ID NO: 86 is an amino acid sequence that has 100% identity with the amino acid sequence having the GenBank accession ID WP_017489914.1. In one or more exemplary embodiment(s), the MFS transporter, expressed according to the present disclosure is Bad. The genetically engineered cell of the present disclosure thus in one or more exemplary embodiment(s) expresses a heterologous MFS transporter protein having at least 80%, such as at least 85%, such as at least 90% such as at least 95% or 100% sequence identity to the amino acid sequence of SEQ ID NO: 86.

In further embodiments, the nucleic acid sequence encoding Bad is under control of a promoter which is recognized by a sigma factor of the cell which is active in the cellular growth-phase, such as RpoD or SigA.

### YberC

The MFS transporter protein identified herein as “YberC protein” or “YberC transporter” or “YberC”, interchangeably, has the amino acid sequence of SEQ ID NO: 87; The amino acid sequence identified herein as SEQ ID NO: 87 is an amino acid sequence that has 100% identity with the amino acid sequence having the GenBank accession ID EEQ08298.1.

In one or more exemplary embodiment(s), the MFS transporter, expressed according to the present disclosure is YberC. The genetically engineered cell of the present disclosure thus in one or more exemplary embodiment(s) expresses a heterologous MFS transporter protein having at least 80%, such as at least 85%, such as at least 90% such as at least 95% or 100% sequence identity to the amino acid sequence of SEQ ID NO: 57.

In further embodiments, the nucleic acid sequence encoding YberC is under control of a promoter which is recognized by a sigma factor of the cell which is active in the cellular growth-phase, such as RpoD or SigA.

### Fred

The MFS transporter protein identified herein as “Fred protein” or “Fred transporter” or “Fred”, interchangeably, has the amino acid sequence of SEQ ID NO: 88; The amino acid sequence identified herein as SEQ ID NO: 88 is an amino acid sequence that has 100% identity with the amino acid sequence having the GenBank accession ID WP_087817556.1.

In one or more exemplary embodiment(s), the MFS transporter, expressed according to the present disclosure is Fred. The genetically engineered cell of the present disclosure thus in one or more exemplary embodiment(s) expresses a heterologous MFS transporter protein having at least 80%, such as at least 85%, such as at least 90% such as at least 95% or 100% sequence identity to the amino acid sequence of SEQ ID NO: 88.

In further embodiments, the nucleic acid sequence encoding Fred is under control of a promoter which is recognized by a sigma factor of the cell which is active in the cellular growth-phase, such as RpoD or SigA.

### Vag

The MFS transporter protein identified herein as “Vag protein” or “Vag transporter” or “Vag”, interchangeably, has the amino acid sequence of SEQ ID NO: 89; The amino acid sequence identified herein as SEQ ID NO: 89 is an amino acid sequence that has 100% identity with the amino acid sequence having the GenBank accession ID WP_048785139.1.

In one or more exemplary embodiment(s), the MFS transporter, expressed according to the present disclosure is Vag. The genetically engineered cell of the present disclosure thus in one or more exemplary embodiment(s) expresses a heterologous MFS transporter protein having at least 80%, such as at least 85%, such as at least 90% such as at least 95% or 100% sequence identity to the amino acid sequence of SEQ ID NO: 89.

In further embodiments, the nucleic acid sequence encoding Vag is under control of a promoter which is recognized by a sigma factor of the cell which is active in the cellular growth-phase, such as RpoD or SigA. Marc

The MFS transporter protein identified herein as “Marc protein” or “Marc transporter” or “Marc”, interchangeably, has the amino acid sequence of SEQ ID NO: 52; The amino acid sequence identified herein as SEQ ID NO: 52 is an amino acid sequence that has 100% identity with the amino acid sequence having the GenBank accession WP_060448169.1.

In embodiments, the MFS transporter, expressed according to the present disclosure is Marc. The genetically engineered cell of the present disclosure thus in one or more exemplary embodiment(s) expresses a heterologous MFS transporter protein having at least 80%, such as at least 85%, such as at least 90% such as at least 95% or 100% sequence identity to the amino acid sequence of SEQ ID NO: 52.

In further embodiments, the nucleic acid sequence encoding Marc is under control of a promoter which is recognized by a sigma factor of the cell which is active in the cellular growth-phase, such as RpoD or SigA.

In embodiments, the expression of the heterologous MFS transporter is regulated by a promoter according to the present invention.

### The Genetically Engineered Cell

In the present context, the terms “a genetically modified cell” and “a genetically engineered cell” are used interchangeably. As used herein “a genetically engineered cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis.

Described herein is a genetically engineered cell for the biosynthetic production of one or more heterologous product(s), wherein
a. the genetically engineered cell comprises at least one heterologous nucleic acid encoding a heterologous product and/or a polypeptide required for the production of the one or more heterologous product,
b. wherein the transcription and/or expression of said heterologous nucleic acid is regulated by a promoter which is recognized by a sigma factor of the cell which is active in the cellular growth-phase, and
c. wherein the function of the sigma factor of the cell which is active in the stationary-phase of the cell is reduced or abolished in said cell.

In embodiments, the genetically engineered cell comprises at least one heterologous nucleic acid involved in the production of the heterologous product, which is controlled by a promoter which is recognized by a sigma factor of the cell which active in the cellular growth-phase, such as RpoD or SigA.

Furthermore, it is understood that the genetic modifications can e.g., be selected from heterologous nucleic acid encoding a heterologous product and/or from enzymes that are required for the production of the one or more heterologous product, such as but not limited to glycosyltransferases, and/or metabolic pathway engineering and specific transporters as well as regulatory factors as described in the above sections, which the skilled person will know how to combine into a genetically engineered cell capable of producing one or more heterologous products.

In a presently preferred embodiment, the genetically engineered cell is capable of producing a heterologous product. In particular a cell free heterologous product.

The genetically engineered cell is preferably a microbial cell, such as a prokaryotic cell. Appropriate microbial cells that may function as a host cell include bacterial cells and archaebacterial cells.

In one preferred embodiment, the genetically engineered cell is a bacterial cell. In a further embodiment the bacterial cell is a gram-negative bacterium. In another embodiment the bacterial cell is a gram-positive bacterium.

### Host Cells

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

In embodiments the microbial strain is not a Salmonella strain attenuated for vaccination purposes.

In one or more exemplary embodiments, the genetically engineered cell is selected from the group consisting of Escherichia coli, Corynebacterium glutamicum, Lactococcus lactis, Bacillus subtilis, Streptomyces lividans.

In one or more exemplary embodiments, the genetically engineered cell is a gram-positive bacterium. In a further embodiment the gram-positive bacterium is selected from the group consisting of Bacillus subtilis, Corynebacterium glutamicum, Lactococcus lactis, Bacillus subtilis, Streptomyces lividans.

In one or more exemplary embodiments, the genetically engineered cell is Bacillus subtilis. In one or more exemplary embodiments, the genetically engineered cell is Corynebacterium glutamicum.

In one or more exemplary embodiments, the genetically engineered cell is a gram-negative bacterium. In a further embodiment the gram-negative bacterium is selected from the group consisting of Escherichia coli and Gluconobacter oxydans.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

In one or more exemplary embodiments, the invention relates to a genetically engineered cell, wherein the cell is derived from the E. coli K-12 DH1 or DH5-a strain.

### Use of a Genetically Engineered Cell

The present invention relates to the use of a genetically engineered cell according to the present invention for producing a heterologous product. In embodiments, the genetically engineered cell is used to produce one or more HMOs as described herein.

In embodiments, the genetically engineered cell is used to produce a polypeptide, such as an antibody, a functional antibody fragment or an enzyme as described herein.

In embodiments, the genetically engineered cell is used to produce a oligosaccharide, such as human milk oligosaccharide as described herein

A method for producing a heterologous product

The present invention relates to a method for producing a heterologous product comprising culturing a genetically engineered cell according to the present invention.

Specifically, the method for producing a heterologous product, comprises,
a. providing a genetically engineered cell as described herein,
b. cultivating the genetically engineered cell in a culture medium under conditions permissive for the production of said a heterologous product; and optionally
c. recovering said a heterologous product from the culture.

In one aspect, the method of the present invention is used for the production of one or more HMO(s) and comprises providing an acceptor saccharide comprising at least two monosaccharide units, which is exogenously added to the culture medium and/or has been produced by a separate microbial fermentation and which is selected form lactose and LNT-II. In a preferred embodiment the substrate for HMO formation is lactose which is fed to the culture during the fermentation of the genetically engineered cell.

The produced heterologous product is retrieved from the culture, either from the culture medium and/or the genetically engineered cell. In embodiments the recovered heterologous product is cell free, meaning no live cells are present in the product and preferably the cells have been removed from the heterologous product.

Culturing or fermenting (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously potentially together with additional ingredients, such as an acceptor oligosaccharide, such as lactose, allowing formation of the HMO product in this phase.

In preferred embodiments the cultivation is a fed-batch fermentation or a continuous fed-batch (feed and bleed fermentation), where the carbon source is continuously feed to the fermentation broth during the fermentation. Preferably the feeding phase is run under carbon limiting conditions.

For the current invention a pH above 6, such as above 6.1, 6.2, 6.3, 6.4, 6.5, 6.6, 6.7, 6.8, 6.9, 7.0, 7.1, 7.2 or 7.3, preferably above 6.5 and below 8. In embodiments, the pH during the cultivation is maintained above 6.0, preferably above 6.5.

The terms “manufacturing” or “manufacturing scale” or “large-scale production” or “large-scale fermentation”, are used interchangeably and in the meaning of the invention defines a fermentation with a minimum volume of 100 L, such as 1000L, such as 10.000L, such as 100.000L, such as 200.000L culture broth. Usually, a “manufacturing scale” process is defined by being capable of processing large volumes yielding amounts of the heterologous product of interest that meets, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon source can for example be selected from the group consisting of glucose, sucrose, fructose, xylose and glycerol. In embodiments, the culturing media is supplemented with one or more carbon sources selected form the group containing glycerol, sucrose and glucose.

In one or more exemplary embodiments, the culturing media contains sucrose as the sole carbon source. In one or more exemplary embodiments, the genetically engineered cell comprises one or more heterologous nucleic acid sequence encoding one or more heterologous polypeptide(s) which enables utilization of sucrose as sole carbon source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

The proteins encoded by the two operons are the sucrose-specific porin (scrY) represented by SEQ ID NO: 53 or a functional homologue thereof, sucrose transport pretein enzyme II (ScrA) represented by SEQ ID NO: 54 or a functional homologue thereof, scrB invertase enzyme (ScrB) represented by SEQ ID NO: 55 or a functional homologue thereof and Scr repressor protein (ScrR) represented by SEQ ID NO: 56 or a functional homologue thereof.

In further embodiments, the nucleic acid sequences encoding the scrYA and scrBR operons are under control of a promoter which is recognized by a sigma factor of the cell which active in the cellular growth-phase, such as RpoD or SigA.

After carrying out the method of this invention, the sialylated HMO produced can be collected from the cell culture or fermentation broth in a conventional manner.

### Retrieving/Harvesting

The heterologous product is retrieved from the culture medium and/or the genetically engineered cell. In the present context, the term “retrieving” is used interchangeably with the term “harvesting”. Both “retrieving” and “harvesting” in the context relate to collecting the produced heterologous product, such as HMOs, from the culture/broth following the termination of fermentation. In one or more exemplary embodiments it may include collecting the heterologous product, such as HMO(s), included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth). In embodiments the heterologous product, such as HMOS, does not include any live cells, preferably, the is separated heterologous product, such as HMOS, is separated from the biomass and therefore cell free.

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification. The heterologous product may be purified according to procedures known by the skilled artesian.

In example, HMOs can be purified according to the procedures known in the art, e.g., such as described in in WO2015/188834, WO2017/182965 or WO2017/152918, wherein the latter describes purification of sialylated HMOs. The purified HMOs can be used as nutraceuticals, pharmaceuticals, or for any other purpose, e.g., for research.

At the end of culturing, the product can be accumulated both in the intra- and the extracellular matrix.

### Biosynthetic Production

The term “biosynthetic production” according to the present invention relates to the production of one or more heterologous products of the present invention, wherein the synthesis/production is carried out by the genetically engineered cell and the product is purified from the cell to obtain a cell free product. As such, biosynthetic production may be a single or multi-step, potentially enzyme-catalyzed process wherein substrates are converted into more complex products in a host organism. For example, in some biosynthesis reactions, simple compounds are modified and/or converted into other compounds, and/or joined to form macromolecules. Accordingly, biosynthetic production often comprises metabolic pathway engineering of the host cell to enable and/or promote the biosynthetic production of the heterologous product. Some of these biosynthetic pathways are located within a single cellular organelle, while others involve enzymes that are located within multiple cellular organelles. Non-limiting examples of metabolic pathway engineering for example relates to engineering of one or more biosynthetic pathways for making at least one sugar nucleotide, engineering of substrate import, engineering pathways for export of the produced heterologous product and/or expression of one or more functional enzymes.

### Manufactured Product

The term “manufactured product” according to the use of the genetically engineered cell or the nucleic acid construct refer to the one or more heterologous products, such as but not limited to HMOs, intended as the one or more products. Various products are described above.

Advantageously, the methods disclosed herein provides an increased overall yield of the product. This, less by-product formation in relation to product formation facilitates an elevated product production and increases efficiency of both the production and product recovery process, providing superior manufacturing procedure of HMOs.

The manufactured product may be a powder, a composition, a suspension, or a gel comprising one or more HMOs.

### Embodiments of the Invention

1. A genetically engineered cell for the biosynthetic production of one or more heterologous product(s), wherein
   a. the genetically engineered cell comprises at least one heterologous nucleic acid encoding a heterologous product and/or a polypeptide required for the production of the one or more heterologous product,
   b. wherein the transcription and/or expression of said heterologous nucleic acid is regulated by a promoter which is recognized by a sigma factor of the cell which is active in the cellular growth-phase, and
   c. wherein the function of the sigma factor of the cell which is active in the stationary-phase of the cell is reduced or abolished in said cell.
2. The genetically engineered cell according to item Error! Reference source not found, wherein the genetically engineered cell is not a vaccine strain.
3. The genetically engineered cell according to item Error! Reference source not found. or 2, wherein the heterologous product is selected from the group consisting of albumin, a peptide, a polypeptide, an antibody and fragments thereof, an enzyme, an oligosaccharide, a vitamin, a cannabinoid and a carotenoid.
4. The genetically engineered cell according to any of any of the preceding items, wherein the heterologous product is not an antigen for vaccination.
5. The genetically engineered cell according to item Error! Reference source not found. to 4, wherein the stationary phase RNA polymerase sigma factor is SigB.
6. The genetically engineered cell according to item Error! Reference source not found. or 4, wherein the stationary phase RNA polymerase sigma factor is RpoS.
7. The genetically engineered cell according to any of any of the preceding items, wherein the cell has been engineered such that the function of a sigma factor of the cell which is active in the stationary-phase of the cell is reduced or abolished.
8. The genetically engineered cell according to any of any of the preceding items, wherein the function of the stationary phase RNA polymerase sigma factor is reduced or abolished by
   a. fully or partially inactivating the gene encoding RpoS or SigB
   b. reducing or abolishing the function of one or more factors promoting the RpoS or SigB function.
9. The genetically engineered cell according to item 8, wherein a gene encoding the one or more RpoS or SigB promoting factor(s) is/are fully or partially inactivated.
10. The genetically engineered cell according any of items 8 or 9, wherein the one or more RpoS promoting factor(s) is/are selected from the group consisting of ArcZ, DksA, GadX, DsrA, DeaD, RprA and Crl.
11. The genetically engineered cell according to any of the preceding items, wherein the function of the stationary phase RNA polymerase sigma factor is reduced or abolished by increasing the function of one or more factor(s) inhibiting the RpoS or SigB function.
12. The genetically engineered cell according to item 11, wherein a nucleic acid encoding the one or more RpoS or SigB inhibitory factor(s) is/are overexpressed.
13. The genetically engineered cell according to any of items 11 to 12, wherein one or more RpoS inhibitory factor(s) is/are selected from the group consisting of rssB, RNase III, H-NS, ArcA, CRP, Fur, MqsA, OxyS and CyaR.
14. The genetically engineered cell according to any one of the preceding items, wherein the transcription of the heterologous nucleic acid is increased when the functionality of the sigma factor of the cell which is active in the stationary-phase of the cell is reduced.
15. The genetically engineered cell according to any of the preceding items, wherein the heterologous nucleic acid is transcribed in the absence of the sigma factor of the cell which is active in the stationary-phase of the cell.
16. The genetically engineered cell according to any of the preceding items, wherein the sigma factor of the cell which is active in the cellular growth-phase is a housekeeping transcription factor such as RpoD or SigA.
17. The genetically engineered cell according to any of the preceding items, wherein the promoter comprises the consensus motif TT (G/C/T) (A/T) C (A/G) (n)₁₄₋₁₈ TA (T/A) (A/G) (A/T) T located at the 5′end region of the heterologous nucleic acid between 5 and 40 nucleotides upstream of the translation start codon AUG.
18. The genetically engineered cell according to any one of the preceding items, wherein the promoter is positively regulated by cAMP-bound CRP.
19. The genetically engineered cell according to item 18, wherein the promotor contains the motif (A/G) TGAnnnnnn (A/T) CAC, located upstream of the translation start codon AUG of the heterologous nucleic acid.
20. The genetically engineered cell according to any of the preceding items, wherein the promoter is selected from the group consisting of PglpF, Plac, PmgIB_70UTR, PglpA_70UTR and PglpT_70UTR (SEQ ID NOs: 35, 44, 32, 33 and 34, respectively) and variants thereof listed in table 1.
21. The genetically engineered cell according to any of the preceding items, wherein the heterologous nucleic acid is selected from the group consisting of one or more glycosyltransferase genes, one or more CMP-N-acetylneuraminic acid pathway genes, one or more transporter genes and one or more nucleic acids encoding the heterologous product to be produced by the genetically engineered cell.
22. The genetically engineered cell according to any of the preceding items, wherein the heterologous product is one or more oligosaccharide(s).
23. The genetically engineered cell according to any of the preceding items, wherein the heterologous product is one or more human milk oligosaccharide(s) (HMO(s)).
24. The genetically engineered cell according to item 23, wherein the human milk oligosaccharide is selected from the group consisting of lacto-N-triose II (LNT-II) lacto-N-tetraose (LNT), lacto-N-neotetraose (LNnT), lacto-N-neohexaose (LNnH), para-lacto-N-neohexaose (pLNnH), para-lacto-N-hexaose (pLNH), lacto-N-hexaose (LNH), 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).
25. The genetically engineered cell according to any of the preceding items, wherein the genetically engineered comprises one or more heterologous nucleic acid encoding one or more heterologous glycosyltransferase.
26. The genetically engineered cell according to item 25, wherein the one or more heterologous glycosyltransferase is selected from the group consisting of β-1,3-N-acetyl-glucosaminyltransferase(s),-1,3-galactosyltransferase(s), β-1,4-galactosyltransferase(s), α-1,2-fucosyltransferase(s), α-1,3-fucosyltransferase, α-2,3-sialyltransferase(s) and α-2,6-sialyltransferase(s).
27. The genetically engineered cell according to any of items 21 to 26, wherein the genetically engineered cell comprises and/or overexpresses a biosynthetic pathway for making at least one sugar nucleotide selected from the group consisting of glucose-UDP-GIcNAc, UDP-galactose, UDP-glucose, UDP-N-acetylglucosamine, UDP-N-acetylgalactosamine (GlcNAc) and CMP-N-acetylneuraminic acid.
28. The genetically engineered cell according to item 27, wherein the genetically engineered cell comprises a de novo GDP-fucose pathway and/or a CMP-N-acetylneuraminic acid pathway.
29. The genetically engineered cell according to any of the preceding items, wherein the cell overexpresses at least one recombinant endogenous gene for which the transcription is regulated by a promoter which is recognized by the RNA transcription factor RpoD, such as a promoter according to item 20.
30. The genetically engineered cell according to item 29, wherein the recombinant endogenous gene is selected from the group consisting of lactose permease, the colanic acid gene cluster and any one of the individual genes of the colanic acid gene cluster selected from manA, manB, manC, gmd and wcaG.
31. The genetically engineered cell according to any of items 22 to 30, wherein the oligosaccharide is a fucosylated oligosaccharide and mannose-6-phosphate isomerase (manA) and/or phosphomannomutase (manB) is overexpressed.
32. The genetically engineered cell according to item 31, wherein the genetically engineered cell comprises at least one additional copy of manB compared to the number of copies of manC (encoding mannose-1-phosphate guanyltransferase) and/or gmd (encoding GDP-D-mannose-4, 6-dehydratase) and/or wcaG (encoding GDP-L-fucose synthase).
33. The genetically engineered cell according to item 31 or 32, wherein the genetically engineered cell comprises at least the same or more copies of manA compared to the number of copies of manC (encoding mannose-1-phosphate guanyltransferase) and/or gmd (encoding GDP-D-mannose-4, 6-dehydratase) and/or wcaG (encoding GDP-L-fucose synthase).
34. The genetically engineered cell according to item 31 to 33, wherein the genetically engineered cell comprises at least two copies of manA and three copies of manB.
35. The genetically engineered cell according to any of the preceding items, wherein the genetically engineered cell comprises and/or overexpresses a biosynthetic pathway for making at least one sugar nucleotide selected from the group consisting of GDP-fucose, UDP-GIcNAc, UDP-galactose, UDP-glucose, UDP-N-acetylglucosamine, UDP-N-acetylgalactosamine (GlcNAc) and CMP-N-acetylneuraminic acid.
36. The genetically engineered cell according to any of the preceding items, wherein the lactose import of the cell is enhanced by expression of one or more lactose permease(s).
37. The genetically engineered cell according to item 36, wherein the lactose permease is LacY, and wherein the nucleic acid sequence encoding LacY is under control of a promoter as defined in any of items Error! Reference source not found. and 17 to 20.
38. The genetically engineered cell according to any of the preceding items, wherein the genetically engineered cell expresses at least one heterologous glycosyltransferase.
39. The genetically engineered cell according to item 38, wherein the heterologous glycosyltransferase is selected from the group consisting of β-1,3-N-acetyl-glucosaminyltransferase(s), β-1,3-galactosyltransferase(s), β-1,4-galactosyltransferase(s), α-1,2-fucosyltransferase(s), α-1,3-fucosyltransferase, α-2,3-sialyltransferase(s) and α-2,6-sialyltransferase(s).
40. The genetically engineered cell according to any of the preceding items, wherein the genetically engineered cell expresses at least one heterologous MFS sugar transporter.
41. The genetically engineered cell according to item 40, wherein the heterologous MFS sugar transporter is selected from the group consisting of Nec, YberC, Fred, Bad and Vag.
42. The genetically engineered cell according to any of the preceding items, wherein the cell further expresses a functional sucrose utilization system.
43. The genetically engineered cell according to item 42, wherein the functional sucrose utilization system is encoded by the scrYA and scrBR operons under control of a promoter which is recognized by a sigma factor of the cell which active in the cellular growth-phase, such as RpoD or SigA.
44. The genetically engineered cell according to any of the preceding items, wherein said engineered cell is a procaryote.
45. The genetically engineered cell according to any of the preceding items, wherein said engineered cell is a gram-negative or gram-positive bacterium.
46. The genetically engineered cell according to item 44 or 45, wherein said procaryote is a bacterium is selected from the group consisting of Escherichia sp., Bacillus sp., lactobacillus sp., corynebacterium sp. and Campylobacter sp.
47. The genetically engineered cell according to item 46 wherein said bacterium is E. coli.
48. The genetically engineered cell according any of the preceding items, wherein the cell has a reduced acetate and/or glutamate formation.
49. A method for biosynthetically producing a heterologous product, wherein said method comprises
   a. providing a genetically engineered cell according to any of items Error! Reference source not found. to 48;
   b. cultivating the genetically engineered cell in a culture medium under conditions permissive for the production of said a heterologous product; and
   c. recovering said a heterologous product from the culture.
50. The method for biosynthetically producing a heterologous product according to item 49, wherein the method comprises cultivating the genetically engineered cell in the presence of a carbon source selected from the group consisting of glucose, sucrose, fructose, xylose and glycerol.
51. The method for biosynthetically producing a heterologous product according to item 49 or 50, wherein the pH during the cultivation is maintained above 6.0, preferably above 6.5.
52. The method for biosynthetically producing a heterologous product according to item 49 to 51, wherein the fermentation is a fed-batch or continuous fed-batch fermentation.
53. The method for biosynthetically producing a heterologous product according to item 49 to 52, wherein the recovered heterologous product is free of living cells.
54. The method according to any of items 49 to 52, wherein the product is one or more human milk oligosaccharides (HMOs).
55. The method according to item 54, wherein lactose is added during the cultivation of the genetically engineered cells as a substrate for the HMO formation.
56. The method according to any of items 54 to 55, wherein the produced human milk oligosaccharide (HMO) is retrieved from the culture medium and/or the genetically engineered cell.
57. Use of a genetically engineered cell according to any of items Error! Reference source not found. to 48, in the production of a heterologous product.
58. The use according to item 57, wherein the heterologous product is cell free.
59. The use according to item 57 or 58, wherein the heterologous product is one or more HMOs.
60. The use according to item 57 or 58, wherein the heterologous product is a polypeptide, such as an antibody or enzyme.
61. The use according to item 57 to 60, wherein the heterologous product is not an antigen for vaccination.

### Sequences

The current application contains a sequence listing in text format and electronical format which is hereby incorporated by reference.

An overview of the SEQ ID NOs used in the present application are shown in Table 2 below.

TABLE 2
Summary of sequences listed in the application

| | | SEQ ID |
|---|---|---|
| Name | Comments | NO |
| rpos | Sigma factor 38 | 1 |
| | Genbank CP001637.1: 1016212-1017204 | |
| | Escherichia coli DH1, | |
| | complete genome | |
| rpoD | Sigma factor 70 | 2 |
| | CP001637.1: 668876-670717 | |
| | Escherichia coli DH1, | |
| | complete genome (reverse) | |
| rpoH | CP001637.1: 282980-283834 | 3 |
| | Escherichia coli DH1, | |
| | complete genome | |
| rpoE | CP001637.1: 1173750-1174325 | 4 |
| | Escherichia coli DH1, | |
| | complete genome | |
| rpoN | CP001637.1: 537614-539047 | 5 |
| | Escherichia coli DH1, | |
| | complete genome (reverse) | |
| rpoF | CP001637.1: 1876266-1876985 | 6 |
| | Escherichia coli DH1, | |
| | complete genome | |
| rpoZ | CP001637.1: 61381-61656 | 7 |
| | Escherichia coli DH1, | |
| | complete genome (reverse) | |
| crl | Sigma factor-binding protein | 8 |
| | CP001637.1: 3613148-3613549 | |
| | Escherichia coli DH1, | |
| | complete genome (reverse) | |
| arcZ | CP001637.1: 533067-533187 | 9 |
| | Escherichia coli DH1, | |
| | complete genome | |
| dksA | CP001637.1: 3710773-3711228 | 10 |
| | Escherichia coli DH1, | |
| | complete genome | |
| gadX | CP001637.1: 217953-218777 | 11 |
| | Escherichia coli DH1, | |
| | complete genome | |
| dsrA | CP001637.1: 1852742-1852832 | 12 |
| | Escherichia coli DH1, | |
| | complete genome | |
| deaD | CP001637.1: 575904-577793 | 13 |
| | Escherichia coli DH1, | |
| | complete genome | |
| rprA | CP001637.1: 2108001-2108106 | 14 |
| | Escherichia coli DH1, | |
| | complete genome | |
| rssB | CP001637.1: 2587223-2588236 | 15 |
| | Escherichia coli DH1, | |
| | complete genome (reverse) | |
| HNS | CP001637.1: 2585556-2585969 | 16 |
| | Escherichia coli DH1, | |
| | complete genome | |
| arcA | CP001637.1: 3872723-3873439 | 17 |
| | Escherichia coli DH1, | |
| | complete genome | |
| crp | CP001637.1: 397012-397644 | 18 |
| | Escherichia coli DH1, | |
| | complete genome (reverse) | |
| fur | CP001637.1: 3170473-3170919 | 19 |
| | Escherichia coli DH1, | |
| | complete genome | |
| msgA | CP001637.1: 715517-715912 | 20 |
| | Escherichia coli DH1, | |
| | complete genome | |
| oxyS | CP001637.1: 4356076-4356192 | 21 |
| | Escherichia coli DH1, | |
| | complete genome | |
| cyaR | CP001637.1: 1710856-1710942 | 22 |
| | Escherichia coli DH1, | |
| | complete genome | |
| PmglB_70UTR_S | Recombinant promoter element | 23 |
| D 8 | | |
| PmglB_70UTR_S | Recombinant promoter element | 24 |
| D 10 | | |
| PmglB-54UTR | Recombinant promoter element | 25 |
| Plac_70UTR | Recombinant promoter element | 26 |
| PmglB_70UTR_S | Recombinant promoter element | 27 |
| D 9 | | |
| PmglB_70UTR_S | Recombinant promoter element | 28 |
| D 4 | | |
| PmglB_70UTR_S | Recombinant promoter element | 29 |
| D 5 | | |
| PglpF_SD4 | Recombinant promoter element | 30 |
| PmglB_70UTR_S | Recombinant promoter element | 31 |
| D 7 | | |
| PmglB_70UTR | Recombinant promoter element | 32 |
| PglpA_70UTR | Recombinant promoter element | 33 |
| PglpT_70UTR | Recombinant promoter element | 34 |
| PglpF | Promoter element from | 35 |
| | Escherichia coli | |
| PglpF_SD10 | Recombinant promoter element | 36 |
| PglpF_SD5 | Recombinant promoter element | 37 |
| PglpF_SD8 | Recombinant promoter element | 38 |
| PmglB_16UTR | Recombinant promoter element | 39 |
| PglpF_SD9 | Recombinant promoter element | 40 |
| PglpF_SD7 | Recombinant promoter element | 41 |
| PglpF_SD6 | Recombinant promoter element | 42 |
| PglpA 16UTR | Recombinant promoter element | 43 |
| Plac | Promoter element from | 44 |
| | Escherichia coli | |
| PglpF_SD3 | Recombinant promoter element | 45 |
| PglpF_SD1 | Recombinant promoter element | 46 |
| futC | ncbi sequence identifier: WP_080473865.1, | 47 |
| | alpha-1,2-fucosyltransferase from | |
| | Helicobacter pylori 26695 | |
| futA | ncbi sequence ID: NP_207177.1, | 48 |
| | alpha-1,3-fucosyltransferase from | |
| | Helicobacter pylori 26695 | |
| d29nst | ncbi sequence ID: AAC44541.1, | 49 |
| | alpha-2,3-sialyltransferase from | |
| | Neisseria meningitidis | |
| ST6,Pd2 | ncbi sequence ID: BAA25316.1, | 50 |
| | alpha-2,6-sialyltransferase from | |
| | Photobacterium damselae | |
| Nec | ncbi sequence ID: WP_092672081.1, | 51 |
| | MFS transporter protein from | |
| | Rosenbergiella nectarea | |
| Marc | ncbi sequence ID: WP_060448169.1, | 52 |
| | MFS transporter protein from | |
| | Serratia marcescens | |
| ScrY | ncbi sequence ID: CAA40657.1, | 53 |
| | ScrY porin protein | |
| | from Klebsiella pneumoniae | |
| ScrA | ncbi sequence ID: CAA40658.1, | 54 |
| | Sucrose transport protein Enzyme II | |
| | scr protein from Klebsiella pneumoniae | |
| ScrB | ncbi sequence ID: WP_000056853.1, | 55 |
| | ScrB invertase enzyme from | |
| | Salmonella enterica subsp. | |
| | enterica serovar Typhimurium | |
| ScrR | ncbi sequence ID: WP_000851062.1, | 56 |
| | Scr Repressor protein from | |
| | Salmonella enterica subsp. | |
| | enterica serovar Typhimurium | |
| CA_cluster | Nucleic acid sequence encoding the | 57 |
| | colanic acid gene cluster from | |
| | Escherichia coli | |
| neuBCA_cluster | Nucleic acid sequence encoding the | 58 |
| | neuBCA cluster from | |
| | Campylobacter jejuni | |
| neuB | ncbi sequence ID: AAK91726.1, Neu5Ac | 59 |
| | synthase coding gene from | |
| | Campylobacter jejuni | |
| neuC | ncbi sequence ID: AAK91727.1, UDP-GIcNAc | 60 |
| | 2-epimerase coding gene from | |
| | Campylobacter jejuni | |
| neuA | ncbi sequence ID: AAK91728.1, CMP-Neu5Ac | 61 |
| | synthetase coding gene from | |
| | Campylobacter jejuni | |
| lacY_HW001_F | RT-qPCR forward primer for LacY | 62 |
| lacY_HW002_R | RT-qPCR reverse primer for LacY | 63 |
| futC_ID0013_F | RT-qPCR forward primer for futC | 64 |
| futC_ID0014_R | RT-qPCR reverse primer for futC | 65 |
| manA_HW075_F | RT-qPCR forward primer for manA | 66 |
| manA_HW076_R | RT-qPCR reverse primer for manA | 67 |
| manB_HW079_F | RT-qPCR forward primer for manB | 68 |
| manB_HW080_R | RT-qPCR reverse primer for manB | 69 |
| gmd_HW071_F | RT-qPCR forward primer for gmd | 70 |
| gmd_HW072_R | RT-qPCR reverse primer for gmd | 71 |
| nec_HW055_F | RT-qPCR forward primer for nec | 72 |
| nec_HW056_R | RT-qPCR reverse primer for nec | 73 |
| scrY_HW087_F | RT-qPCR forward primer for scrY | 74 |
| scrY_HW088_R | RT-qPCR reverse primer for scrY | 75 |
| crp_HW063_F | RT-qPCR forward primer for crp | 76 |
| crp_HW064_R | RT-qPCR reverse primer for crp | 77 |
| cra_HW083_F | RT-qPCR forward primer for cra | 78 |
| cra_HW084_R | RT-qPCR reverse primer for cra | 79 |
| cysG_ID0032_F | RT-qPCR forward primer for cysG | 80 |
| cysG_ID0033_R | RT-qPCR reverse primer for cysG | 81 |
| PgatY | Promoter element from Escherichia coli | 82 |
| PglpA | Promoter element from Escherichia coli | 83 |
| PmglB | Promoter element from Escherichia coli | 84 |
| LacY | Lactose permease LacY, | 85 |
| | GenBank ID: NP_414877.1, | |
| Bad | GenBank accession ID WP_017489914.1, | 86 |
| | MFS transport protein | |
| YberC | GenBank accession ID EEQ08298.1, | 87 |
| | MFS transport protein | |
| Fred | GenBank accession ID WP_087817556.1, | 88 |
| | MFS transport protein | |
| Vag | GenBank accession ID WP_048785139.1, | 89 |
| | MFS transport protein | |
| RpoS | RNA polymerase sigma factor 38 subunit, | 90 |
| | Genbank BAA02747.1 | |
| RpoD | RNA polymerase sigma factor70 subunit, | 91 |
| | Genbank ID ACX38320.1 | |
| Crl | RNA polymerase holoenzyme assembly | 92 |
| | factor Crl, Genbank ID ACX40987.1 | |
| sigB | NC_000964.3:522862-523650 | 93 |
| | Bacillus subtilis subsp. | |
| | subtilis str. 168 complete genome | |
| SigB | RNA polymerase sigma factor B, | 94 |
| | Genbank ID WP_003246715.1 | |
| sigA | Sigma factor A encoding sequence, | 95 |
| | GenBank ID: AB012852.1 | |
| sigA | RNA polymerase sigma factor A, | 96 |
| | GenBank ID: BAA25730.1 | |
| DskA | transcriptional regulator, TraR/DksA | 97 |
| | family, GenBank ID: ACX41072.1 | |
| GdaaX | transcriptional regulator, AraC family, | 98 |
| | GenBank ID: ACX37891.1 | |
| DeaD | DEAD/DEAH box helicase domain protein, | 99 |
| | GenBank ID: ACX38231.1 | |
| RssB | regulator of RpoS, GenBank ID: GHL23200.1 | 100 |
| HNS | histone family protein nucleoid-structuring | 101 |
| | protein H-NS, GenBank ID: CX40054.1 | |
| ArcA | two component transcriptional regulator, | 102 |
| | winged helix family, GenBank ID: ACX41209.1 | |
| Crp | transcriptional regulator, Crp/Fnr family, | 103 |
| | GenBank ID: ACX38045.1 | |
| Fur | ferric uptake regulator, Fur family, | 104 |
| | GenBank ID: ACX40583.1 | |
| MqsA | transcriptional regulator, XRE family, | 105 |
| | GenBank ID: ACX38365.1 | |
| RNaseIII | CP001637.1:1179699-1180379 | 106 |
| | Escherichia coli | |
| | DH1, complete genome | |
| RNaseIII | Ribonuclease III (RNAselll), | 107 |
| | GenBank ID: ACX38775.1 | |
| PgatY_70UTR | Recombinant promoter element | 108 |
| PgatY_50UTR | Recombinant promoter element | 109 |
| PgatY_16UTR | Recombinant promoter element | 110 |
| manA | CP001637.1:2188727-2189902 | 111 |
| | Escherichia coli | |
| | DH1, complete genome | |
| ManA | mannose-6-phosphate isomerase, class I, | 112 |
| | GenBank ID: ACX39687.1 | |
| manB | CP001637.1:1755077-1756447 | 113 |
| | Escherichia coli | |
| | DH1, complete genome | |
| ManB | Phosphomannomutase, | 114 |
| | GenBank ID: ACX39274.1 | |

### Examples

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J.H. Experiments in molecular genetics (1972.) (Cold spring Harbor Laboratory Press, NY)

The examples described below are selected to illustrate the invention and are not limiting the invention in any way.

### Strains

The E. coli strains (genetically engineered cells) constructed in the present application were based on Escherichia coli K-12 DH1 with the genotype: F″, A, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting or deleting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186:2673-81 and Warming et al 2005 Nucleic Acids Res. 33 (4): e36) with specific selection marker genes and screening methods.

The MDO strain was further engineered to generate different HMO producing strains.

The genotypes of the background strain (MDO), and the different HMO strains are shown in Table 12 below.

TABLE 12
genotypes of strains used in the examples

| Strain | | | |
|---|---|---|---|
| ID | Product | Modification | Genotype |
| MDO | None | | F- λ- ΔendA1 ΔrecA1 ΔrelA1 ΔgyrA96 |
| | | | Δthi-1 glnV44 hsdR17(rK-mK-) ΔlacZ wcaF::Plac |
| | | | ΔnanKETA ΔlacA ΔmelA ΔwcaJ ΔmdoH |
| Strain 1 | 2′FL | | MDO 2xPglpF-futC¹ PglpF-CA² PglpF-nec³ Pscr-scrBR⁴ |
| | | | PglpF_SD1-scrYA⁴ |
| Strain 2 | 2′FL | ΔrpoS | MDO 2xPglpF-futC¹ PglpF-CA² PglpF-nec³ Pscr-scrBR⁴ |
| | | | PglpF_SD1-scrYA4 ΔrpoS |
| Strain 3 | DFL | | MDO wcaF::PglpF 3xPglpF-futA⁵ PglpF-marc⁶ |
| | | | PmglB_70UTR_SD4-futC¹ |
| Strain 4 | DFL | ΔrpoS | MDO wcaF::PglpF 3xPglpF-futA⁵ PglpF-marc⁶ |
| | | | PmglB_70UTR_SD4-futC1 ΔrpoS |
| Strain 5 | 3FL | | MDO wcaF::PglpF 3xPglpF-futA⁵ PglpF-marc⁶ |
| Strain 6 | 3FL | ΔrpoS | MDO wcaF::PglpF 3xPglpF-futA⁵ PglpF-marc⁶ ΔrpoS |
| Strain 7 | 3′SL | | MDO 2xPlac-Δ29nst⁷ ΔnadC⁸ pBS-nadC-Plac-neuBCA⁹ |
| Strain 8 | 3′SL | ΔrpoS | MDO 2xPlac-Δ29nst ΔnadC⁸ pBS-nadC-Plac-neuBCA⁹ |
| | | | ΔrpoS |
| Strain 9 | 6′SL | | MDO PglpF-neuB¹⁰ PglpF-neuC¹⁰ PglpF-neuA¹⁰ |
| | | | PglpF-ST6, Pd2¹¹ |
| Strain 10 | 6′SL | ΔrpoS | MDO PglpF-neuB¹⁰ PglpF-neuC¹⁰ PglpF-neuA¹⁰ |
| | | | PglpF-ST6, Pd2¹¹ ΔrpoS¹² |
| Strain 11 | 2′FL | Δcri | MDO 2xPglpF-futC¹ PglpF-CA² PglpF-nec³ |
| | | | Pscr-scrBR⁴ PglpF_SD1-scrYA⁴ Δcrl¹³ |
| Strain 12 | 2′FL | ΔrpoZ | MDO 2xPglpF-futC¹ PglpF-CA² PglpF-nec³ Pscr-scrBR⁴ |
| | | | PglpF_SD1-scrYA⁴ ΔrpoZ¹⁵ |
| Strain 113 | 2′FL | ΔrpoF | MDO 2xPglpF-futC¹ PglpF-CA² PglpF-nec³ Pscr-scrBR⁴ |
| | | | PglpF_SD1-scrYA⁴ ΔrpoF¹⁶ |
| Strain 14 | 2′FL | ΔrpoS | MDO 2xPglpF-futC¹ PglpF-CA² PglpF-nec³ Pscr-scrBR⁴ |
| | | manB¹⁷+ | PglpF_SD1-scrYA⁴ ΔrpoS PglpF-manB PglpF-manA |
| | | manA¹⁸+ | |

¹futC - gene encoding alpha-1,2-fucosyl-transferase of SEQ ID NO: 47.
²CA = extra colanic acid gene cluster (gmd-wcaG-wcaH-wcal-manC-manB, SEQ ID NO: 57) under the control of a PglpF promoter at a locus that is different than the native locus.
³nec gene coding for a heterologous major facilitator superfamily (MFS) transporter of SEQ ID NO: 51.
⁴scrYA, scrBR two operons encoding the sequences of SEQ ID NO: 53 and 54 and SEQ ID NO: 55 and 56 respectively.
⁵futA - gene encoding a-1,3-fucosyl-transferase of SEQ ID NO: 48.
⁶marc gene coding for a heterologous major facilitator superfamily (MFS) transporter of SEQ ID NO: 52.
⁷Δ29nst - gene encoding α-2,3-sialyltransferase of SEQ ID NO: 49.
⁸ΔnadC - deletion of quinolinate phosphoribosyl-transferase of WP_101348535.1 for further details see WO2017101958.
⁹pBS-nadC-Plac-neuBCA - plasmid expressing neuBCA (SEQ ID NO: 58) and nadC further details see WO 2017/101958.
¹⁰neuB, neuC, neuA - codon optimized genes encoding NeuB, NeuC and Neu A of SEQ ID NO 59, 60, 61, respectively.
¹¹ST6, Pd2 - gene encoding a2,6-sialyltransferase of SEQ ID NO: 50.
¹²ΔrpoS deletion of sigma factor 38 gene of SEQ ID NO: 1, encoding the sequence of SEQ ID NO: 90.
¹³Δcrl deletion of the sigma factor binding protein Crl gene of SEQ ID NO: 8, encoding the sequence of SEQ ID NO: 92.
¹⁴wcaF::PglpF -PglpF promoter inserted in front of the native colonic acid gene cluster (gmd-wcaG-wcaH-wcal-manC-manB).
¹⁵ΔrpoZ deletion of the rpoZ sigma factor gene of SEQ ID NO: 7.
¹⁶ΔrpoF deletion of the sigma factor 28 gene of SEQ ID NO: 6.
¹⁷manB insertion of a nucleic acid encoding manB (SEQ ID NO: 113) encoding the protein phosphomannomutase (SEQ ID NO: 114).
¹⁸manA insertion of a nucleic acid encoding manA (SEQ ID NO: 111) encoding the protein mannose-6 phosphate isomerase (SEQ ID NO: 112).

### Deep Well Assay for Product Formation

The strains were screened in 96 deep well plates using a 3-day protocol for assessment of

HMO production. During the first 24 hours, fresh precultures were grown to high densities. More specifically, fresh precultures were prepared using a basal minimal medium supplemented with magnesium sulphate, thiamine and glucose. The pH was adjusted to pH 7.0 with NaOH. The precultures were incubated for 24 hours at 34° C. and 1000 rpm shaking.

For HMO production an aliquot of the pre-culture was transferred to a new deep well plate with basal minimal medium (BMM7, pH 7,5) to start the main culture. The new BMM was supplemented with thiamine and magnesium sulphate, 0.01% of glucose, 2% lactose, 1.75% of Maltodextrin, thiamine, and Glucoamylase for optimized hydrolysis of maltodextrin. The HMO main cultures were incubated for 48 hours at 28° C. and 1000 rpm shaking. The assay was generally performed in triplicates.

After incubation of the main cultures were boiled and centrifuged. Samples were analysed by HPLC (2′FL, DFL and 3FL) or HPAEC (3′SL and 6′SL).

### Deep Well Assay for Acetate Assessment

The strains were screened in 96 deep well plates using a 2-day protocol with high glucose for acetate assessment. During the first 24 hours, fresh precultures were grown to high densities and subsequently transferred to a medium that allowed for induced acetate formation. More specifically, fresh precultures were prepared using a basal minimal medium supplemented with magnesium sulphate, thiamine and glucose The pH was adjusted to pH 7.0 with NaOH. The precultures were incubated for 24 hours at 34° C. and 1000 rpm shaking.

For acetate production, an aliquot of the of the pre-culture was transferred into new deep-well plates, with BMM medium (pH 7.5) with thiamine and magnesium sulphate and 2.5% of glucose. The acetate main cultures were incubated at 30° C., 1.000 rpm for 24 hours. Samples were collected by centrifugation of the broth at 10.000 rpm for 3 minutes. Acetate concentrations were measured from the supernatant using the Acetic Acid Assay Kit (Megazyme) following the guidelines of the manufacturer.

### Fed-Batch Fermentation

The E. coli strains were cultivated in Sartorius Biostat B 2L bioreactor systems starting with 700 g of mineral culture basal medium consisting of 6.9 g/kg carbon source, (glucose for the 3FL fermentations in example 5 or sucrose for the 2′FL fermentations in example 6), lactose, H₃PO₄, MgSO₄×7H₂O, KOH, citric acid, trace element solution, antifoam and thiamine. The dissolved oxygen level was kept at 23% by a cascade of first agitation and then airflow (as safeguard) starting at 1000 rpm (up to max 2000 rpm) and 1 WVM (up to max 3 WVM). The pH was kept at 6.8 by titration with 10% NH₄OH solution. The cultivations were started with 2% (v/v) inoculums from shake-flask pre-cultures comprising of 20 g/L carbon source, (NH₄)₂HPO₄, KH₂PO₄, MgSO₄×7H₂O, KOH, NaOH, citric acid, trace element solution, antifoam and thiamine, adjusted to pH 7, that had been grown at 33° C., 200 rpm until they had reached an optical density at 600 nm (OD600) of 2.5-4. After the initial batch growth phase and the depletion of the carbon source contained in the basal medium, a concentrated feed solution containing the same components as in the basal medium was fed starting at 1g carbon source/h and following a profile that ramped up at a rate that kept the cultivation carbon limited and that avoided the triggering of any overflow metabolism in the strain or oxygen limitation in the vessel. The temperature was initially set to 33° C. but was dropped to 32° C. with a 1h ramp after the feed rate had peaked. The growth and metabolic activity and state of the cells were followed by on-line measurements of CO₂ evolution rate, Oxygen uptake rate, agitation, pH, temperature, base titrant addition and dissolved oxygen level. Throughout the fermentation, samples were taken in order to determine the concentration of 2′-FL, 3FL. DFL, lactose, glutamic acid and other minor by-products using HPLC. Total broth samples were diluted three-fold in deionized water and boiled for 20 minutes. This was followed by centrifugation at 17000 g for 3 minutes, whereafter the resulting supernatant was analysed by HPLC. In addition, OD600 and biowetmass, defined as the weight fraction of the pellet in the broth (in g/kg) after 3 min centrifugation at 17000 g, were measured to follow the growth of the strain. Acetic acid was measured using Acetic Acid Assay Kit (Megazyme).

### RT-qPCR

Total RNA from E. coli was prepared using RNeasy MiniKit (Qiagen) according to the manufacturer's instructions. DNA digestion was performed on-column during RNA purification using the RNase-Free DNase set (Qiagen). The integrities of all RNA samples were confirmed using the Agilent 4150 TapeStation System (Agilent Technologies) and RNA concentration measure using Nanodrop (ThermoFisher Scientific). The RT-PCR was done using 50 ng of total RNA sample for a 20 μl reaction including, primers (MWG Eurofins), Power SYBR Green RNA-to-CT 1-Step Kit (Applied Biosystems). The RT-PCR was run on QuantStudio 5 instrument (Applied Biosystems) according to the manufacturer's instructions and PCR products were detected with SYBR Green dye. Results were analyzed using the Design and Analysis Software v1.5.1 (Thermo Scientific), where amplification of the target gene was normalized using the endogenous control (cysG), and the relative quantity of target determined by comparing normalized target quantity in each sample to normalized target quantity in the reference sample.

Determination of Primer Efficiency by Quantitative PCR (qPCR)

The efficiency of primer couples was determined by generating standard curve. Standard samples were prepared by PCR reactions and product purified by QIAquick PCR purification kit (QIAGEN). For each standard, six points of a 10-fold dilution series were prepared for PCR reaction using the Fast SYBR Green Master Mix (Thermo Scientific) and run in three technical replicates on the QuantStudio 5 qPCR instrument (Applied Biosystems). Results were analyzed using the Design and Analysis Software v1.5.1 (Thermo Scientific) and the primer efficiency determined.

The primers used are listed in table 13.

TABLE 13
RT-qPCT primers:

| | | | | SEQ |
|---|---|---|---|---|
| | Primer | Direc- | Primer sequence | ID |
| Target | name | tion | (5′-3′) | NO |
| lacY | HW001 | F | GGGAGCCTACTTCCCGTTTT | 62 |
| lacY | HW002 | R | GCCCGAGTTTGTCAGAAAGC | 63 |
| futC | ID0013 | F | CGGTTGTCAGCTGGGTATTG | 64 |
| futC | ID0014 | R | CGCGTGGTCATATCCATGAAC | 65 |
| manA | HW075 | F | GCCATTTTGTTCTGCGTCGA | 66 |
| manA | HW076 | R | GCCTTTGACAGTCACCGGT | 67 |
| manB | HW079 | F | GCCCACCATTACTTCCGTGA | 68 |
| manB | HW080 | R | CTGTTGATCTCACCGCTTGC | 69 |
| gmd | HW071 | F | CGTCCGGCTGAAGTTGAAAC | 70 |
| gmd | HW072 | R | CGCTTCGAGGTCATTAGCCA | 71 |
| nec | HW055 | F | GGGTCTGGTTATGCTGTGGT | 72 |
| nec | HW056 | R | CGTAAATCGCTTGGTGACCC | 73 |
| scrY | HW087 | F | CCCTCAACCTGCGTCTGATT | 74 |
| scrY | HW088 | R | TAGAAGCTGCCATTGACCGC | 75 |
| crp | HW063 | F | CAGACGCTATGACTCACCCG | 76 |
| crp | HW064 | R | TGTGCGGAGATCAGGTTCTG | 77 |
| Cra | HW083 | F | AAGGTTTCCGTACTGCCTGG | 78 |
| cra | HW084 | R | ATCACTCCTTGCAACAACGC | 79 |
| cysG | ID0032 | F | GTATTCCACTCACGCATCGC | 80 |
| cysG | ID0033 | R | ATAGTCGCGGCCTGATTCAA | 81 |

### Proteomics Analysis

Fermentation broth was sampled directly into a 3 mL ice cold 0.9% NaCl solution diluting the broth 3-fold. Cells were harvested by centrifugation at 4000 g for 10 min at 4° C. The pellet was washed in ice cold 0.9% NaCl solution and the suspension was centrifuged again for 5 min at 6000 g at 4° C. The supernatant was removed, and the pellet was immediately placed on dry ice and was stored at −80° C. until further analysis. The samples were normalized for their biomass concentration, based on biomass measurements, and subsequently lysed by adding lysis buffer (PreOmics) and incubated at 95° C. for 20 minutes. Cell lysates were processed further by reduction, alkylation, and digestion using trypsin. The applied method was a 20-min gradient Data-Independent Acquisition Label-Free Quantitation (DIA LFQ) proteomics. Samples were analyzed in technical triplicates by liquid chromatography tandem mass spectrometry (LC-MS/MS) using a Vanquish UHPLC coupled to a Q Exactive Plus Orbitrap MS (Thermo Fisher Scientific). Peptides were separated using reverse-phase chromatography using a gradient of water with 0.1% formic acid (solvent A) and 20% water and 0.1% formic acid in acetonitrile (solvent B) from 5% B to 40% B in 20 min. Data-independent acquisition (DIA) was performed with a resolution setting at 17,500 within the 400—to 1,200 m/z range and a maximum injection time of 20 ms, followed by high-energy collision-induced dissociation activated (HCD) MS/MS. Eight MS/MS windows of 60 Da were used, ranging from 400 to 873 m/z using a resolution setting of 17,500 with maximum injection time set to automatic. Raw data was analyzed with the Pulsar search engine and Spectronaut (version 14.10), against the proteins of Strain 1 and Strain 2 allowing Trypsin/P specific peptides including 2 missed cleavages, an oxidation on methionine, carbamidomethylated cysteines, and deamidated asparagine and glutamine. Label-free quantification was performed using the top three unique peptides measured for each protein. Retention time alignment was performed on the most abundant signals obtained from peptides measured in all samples, and results were filtered by FDR of 1% followed by normalization of the result using the median ion intensities measured for each sample.

### Example 1

Testing Effect of Sigma Factor Deletion in HMO Producing Escherichia coli

In the present example it was investigated if deletion of different sigma factors had an effect on HMO production in E. coli. The E. coli chromosome encodes seven sigma factors needed for initiation of transcription. Three sigma factors are essential (RpoD, RpoH, and RpoE) for cell growth.

2′FL producing strains corresponding to Strain 1 in table 12 above were generated where each strain had either rpoN, rpoF (strain 13), rpoZ (strain 12) or rpoS (strain 2) deleted. The cells were screened for 2′FL production in the deep well assay for product formation described in the method section above.

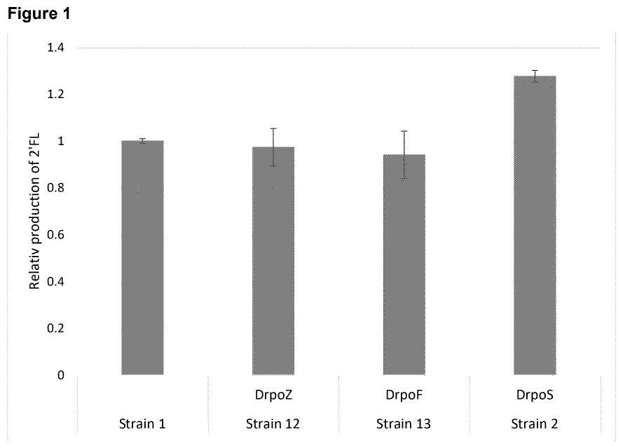

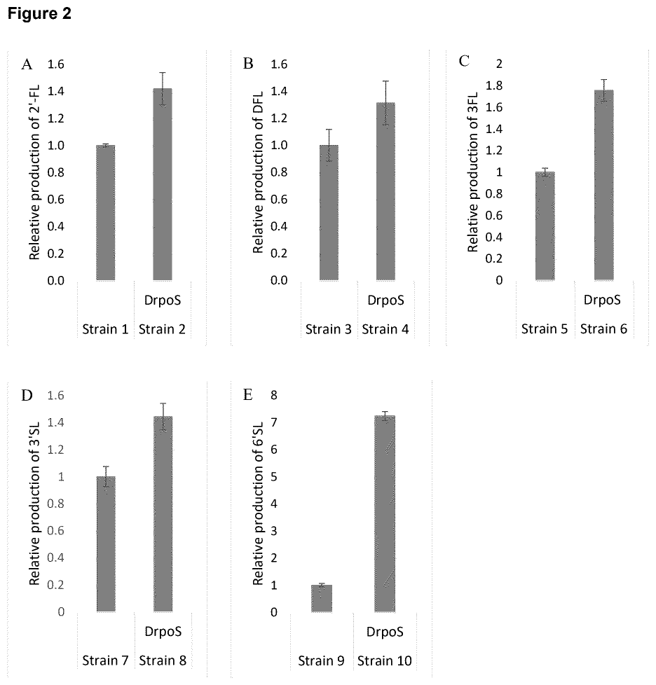

From this sigma factor screening it was observed that deletion of the gene encoding sigma factor RpoN resulted in no growth in minimal media supplied with glucose. Removal of the genes encoding either sigma factor RpoF or RpoZ gene did not affect HMO production (FIG. 1). Surprisingly, removing the gene encoding sigma factor RpoS in the 2′FL expressing E. coli strains increased production of 2′FL (see also FIG. 1 and FIG. 2A)

### Example 2

Effect of Deletion of rpoS in Different HMO Producing Escherichia coli Strains

The present example sets out to investigate if the positive effect on 2′FL production was also observed for strains producing other HMO's.

Specifically, the following strains 2′FL (strain 1 and 2, example 1), DFL (Strain 3 and 4), 3FL (strain 5 and 6), 3′SL (strain 7 and 8) and 6′SL (strain 9 and 10) were generated. The genotype of the individual strains is shown in table 12 in the method section above.

All the strains were screened according to the deep well assay for product formation described in the method section. The results are shown in FIG. 2 as the relative expression of the HMO strain+rpoS strain (set to 1).

From this it can be seen that deletion of rpoS in a 2′FL producing E. coli strain increased the 2′FL titer with 40% (FIG. 2A). Deletion of sigma factor rpoS in a E. coli DFL production strain increased the DFL titer with 30% (FIG. 2B). Deletion of sigma factor rpoS in a E. coli 3FL production strain increased the 3FL titer with 70% (FIG. 2C). Deletion of sigma factor rpoS in a E. coli 3′SL production strain increased the 3′SL titer with 40% (FIG. 2D). Deletion of sigma factor rpoS in a E. coli 6′SL production strain increased the 6′SL titer with 700% (FIG. 2E).

### Example 3

Engineering Escherichia coli for Increased HMO Production by Deleting Clr

The level of RpoS in the cell is regulated by several transcription and translational factors. Hence, modulation of transcription and/or translation of rpoS may reduce the level of RpoS and thereby increase production of HMOs in an E. coli HMO producing strain.

In the present example it was therefore investigated whether instead of deleting rpoS, an alternative modulation of the activity of RpoS could be deleted and give similar effects. One example of a RpoS modulator is the sigma factor-binding protein, Crl, that promotes the binding of RpoS to the RNA polymerase, thereby activating expression of RpoS regulated genes.

Deletion of the gene encoding Crl, crl, in an HMO producing E. coli strain is expected to i) reduced transcription of RpoS-regulated genes and ii) have a positive effect on HMO production.

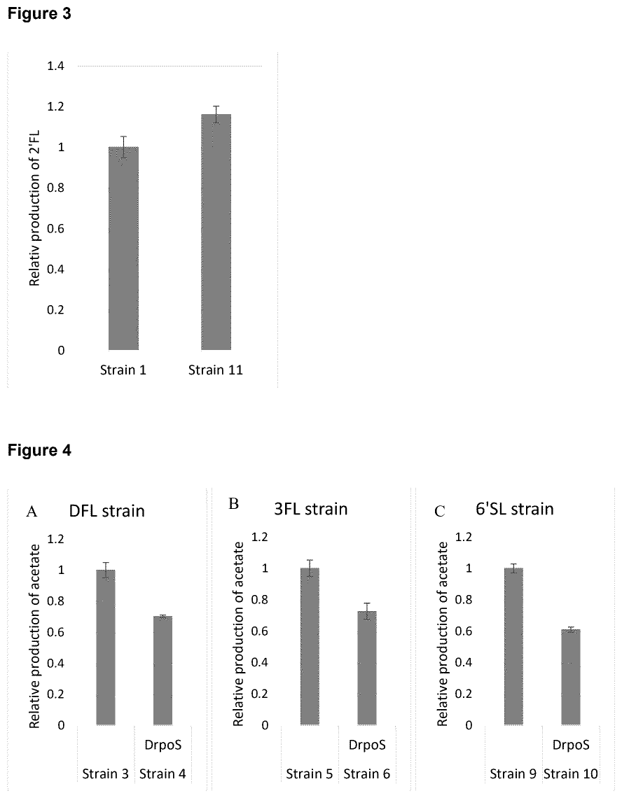

In the present example the cri gene was deleted in the 2′FL expressing strain 1, thereby generating strain 11. The strains were screened as described in example 1. The result is shown in FIG. 3 as the relative expression of strain 1 which does not contain the rpos deletion (set to 1).

From this is can be seen that deletion of crl in an E. coli 2′FL production strain (Strain 11) increased the 2′FL titer with 20% compared to a 2′FL production strain with an intact crl gene (FIG. 2).

### Example 4

Decreased Acetate Formation in E. coli HMO Production Strains Lacking rpoS

E. coli generates acetate when grown aerobically on glucose. Minimizing acetate formation in E. coli fermentations is therefore desirable since acetate have negative effects on growth and protein production.

In the present example it was investigated whether a difference in acetate formation could be observed in the DFL (Strain 3 and 4), 3FL (strain 5 and 6) and 6′SL (strain 9 and 10) strains when rpoS was deleted. The strains were screened using the deep well assay for acetate assessment described in the method section above. The results are shown in FIG. 4 as the relative amount of acetate in the Rpos positive strain (set to 1)

From this it can be seen that deletion of the gene encoding RpoS in E. coli, rpoS, reduced the formation of acetate in all three HMO producing strains. For instance, removal of rpoS in a DFL producing E. coli strain reduced the relative acetate formation with 30% following overnight growth in minimal media supplied with glucose (FIG. 4A). Similarly, acetate formation was reduced by 30% or 40% in E. coli strains producing either 3FL (FIG. 4B), or 6′SL (FIG. 4C), respectively, when rpoS was removed.

### Example 5

Fermentation Using an E. coli 3FL Production Strain Lacking Rpos

In the present example it was investigated whether the increased HMO formation and reduced acetate formation observed in example 4 in the deep well assays could also be observed in a fed-batch fermentation.

The 3FL strain 5 (+rpoS) and strain 6 (ΔrpoS) were fermented as described in the method section using glucose as carbon source.

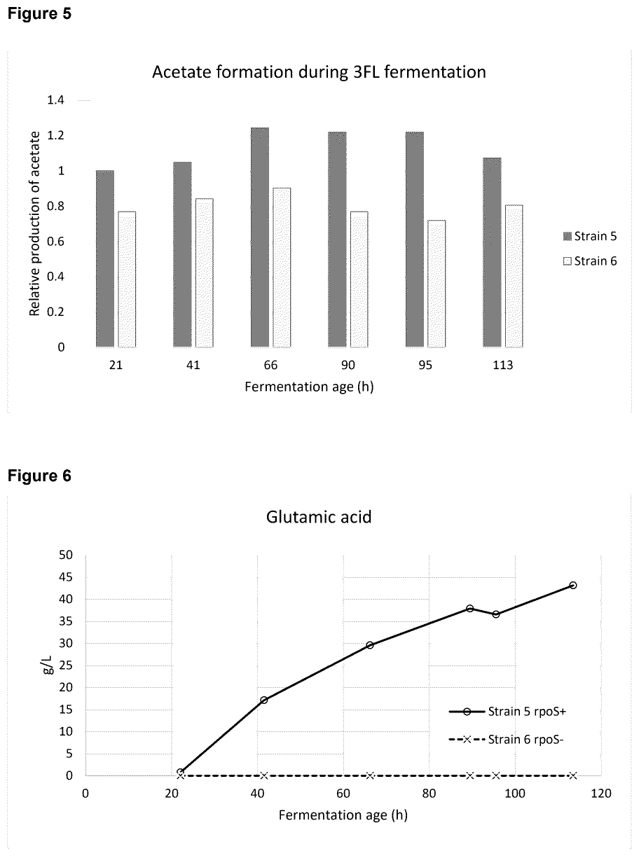

FIG. 5 shows that deletion of rpoS in a 3FL producing E. coli strain reduced the relative acetate formation between 25-45% during fermentation. Samples were collected at six different timepoints after feed start (21, 41, 66, 90, 95, 113 hours) and the amount of acetate was measured using the megazyme kit. The amount of acetate is given as relative values compared to Strain 5 at timepoint 21 hours (FIG. 5).

Likewise, the amount of glutamate was measured using HPLC. The results are shown in FIG. 6, and clearly show that deletion of rpoS in a 3FL producing E. coli strain (strain 6) results in non-quantifiable levels of glutamate formation as compared to high levels accumulating in the rpoS positive strain (strain 5).

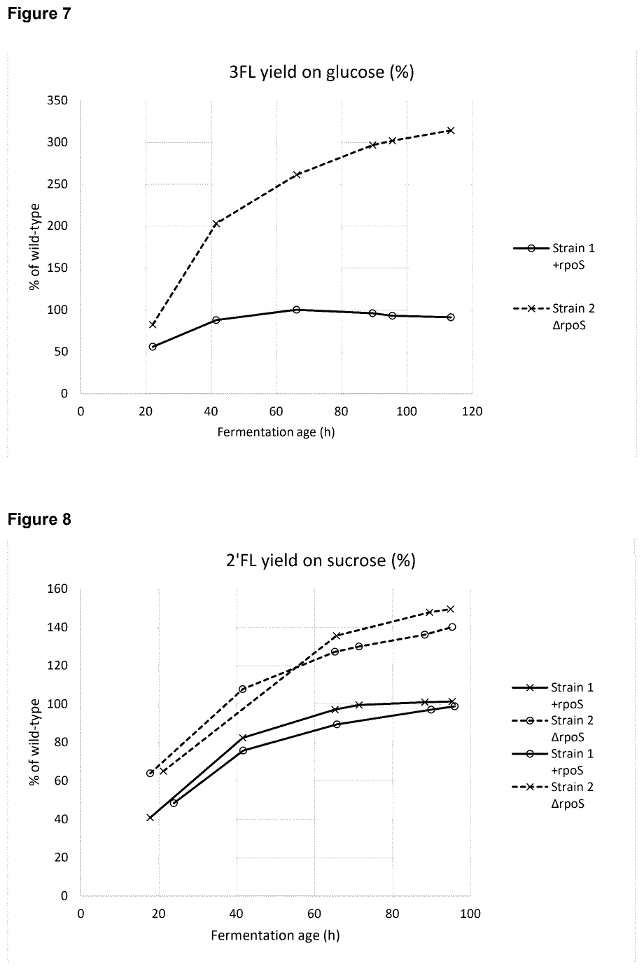

FIG. 7 likewise show that deletion of sigma factor rpoS in a E. coli 3FL production strain increased the 3FL yield to 300% at the end of fermentation. The yield was measured as the accumulated yield of product per consumed carbon source substrate (Yps) (g 3FL/g glucose) and was normalized to the Yps of strain 5 (+rpoS) at the end of fermentation (set to 100%).

### Example 6

Increased 2′FL Production in Fermentation by Deleting rpoS in E. coli

In the present example it was investigated whether the increased 2′FL formation observed in example 1 and 2 in the deep well assays could also be observed in a fed-batch fermentation.

The 2′FL strain 1 (+rpoS) and strain 2 (ΔrpoS) were fermented in duplicate as described in the method section using sucrose as carbon source.

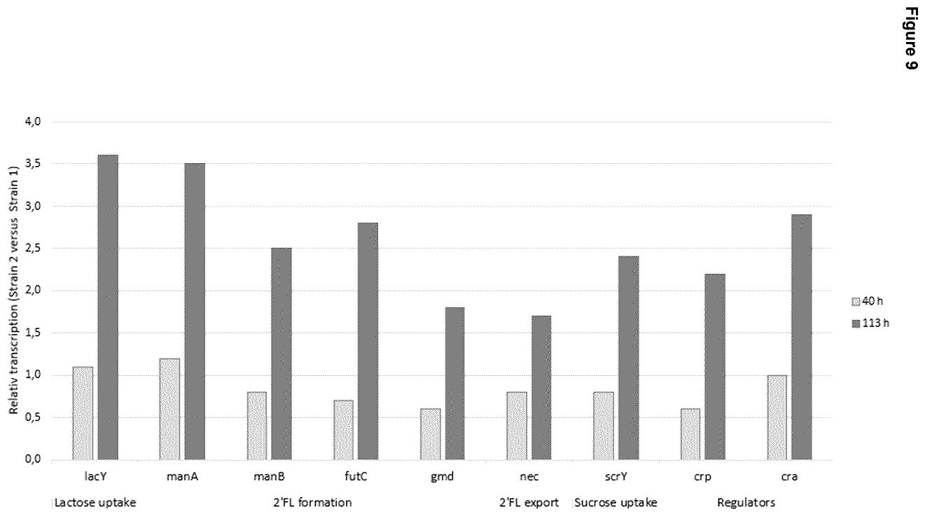

FIG. 8 shows that deletion of rpoS in a 2′FL producing E. coli strain increased the 2′FL yield in fermentation to 140%. Samples were collected at six different timepoints after feed start (21, 41, 66, 90, 95, 113 hours) and the amount of 2′FL was measured. The yield was measured as accumulated product yield per carbon source substrate (Yps) (g 2′FL/g sucrose) and was normalized to the Yps of strain 1 (+rpoS) at the end of fermentation.

### Example 7

Deletion of rpoS Increase Transcription of Genes Involved in Lactose Uptake, GDP-Fucose Formation, Fucosylation, and 2′FL Export in E. coli 2′FL Production Strain

In the present example it was investigated how the deletion of rpoS affected the expression of genes that are relevant for HMO expression.

Specifically, in the fermentation described in example 6, samples were collected at different timepoints after feed start (41 and 113 hours) and the transcription levels of 9 selected target genes (see table 13) were determined using RT-qPCR as described in the methods section above.

TABLE 13
selected target genes

| | | Relevance in |
|---|---|---|
| Gene | Function | HMO production |
| lacY | Lactose permease | Lactose uptake |
| futC | Fucosyltransferase needed for | 2′FL formation |
| | decoration of lactose to form 2′FL | |
| manA | Part of colonic acid pathway | |
| manB | responsible for GDP-fucose | |
| gmd | | |
| nec | major facilitator superfamily | Export of 2′FL out of |
| | (MFS) transporter | the cell |
| scrY | sucrose specific ScrY porin | Import of sucrose |
| crp | Cyclic AMP receptor protein | Transcriptional regulator |
| cra | catabolite repressor/activator | |

FIG. 9 shows the relative transcription level of Stain 2 (ΔrpoS) over Strain 1 (+rpoS). Specifically, values above 1 indicates that the respective gene is transcribed at a higher level in strain 2 than in strain 1.

Deletion of rpoS in the 2′FL producing E. coli strain affected the transcriptional level of several genes. The relative transcription levels between Stain 2 (ΔrpoS) and Strain 1 (rpoS+) at time 41 hours are shown in light gray bars (FIG. 9). The relative transcription levels between Stain 2 (ΔrpoS) and Strain 1 (+rpoS) at time 113 hours are shown in dark gray bars (FIG. 9). Removal of rpoS increased transcription of genes involved in lactose uptake, 2′FL formation, 2′FL export, sucrose uptake, and global regulators 2-4-fold.

### Example 8

Increased Heterologous Protein Expression in E. coli Strain Lacking rpoS

In the present example it was investigated how the deletion of rpoS affected the expression of heterologous proteins that are relevant for HMO expression. Membrane bound proteins were excluded from the analysis since they may be difficult to access accurately using the proteomic analysis described in the methods above.

Specifically, in the fermentation described in example 6, where an E. coli 2′FL production strains with either an intact rpoS gene (Strain 1, rpoS_pos) or lacking rpoS (Strain 2, rpoS_neg) was fermented in a fed-batch fermentation, the samples collected at different timepoint after feed start (21, 41, 66, 90, 95, 113 hours) were analyzed for the amount of selected proteins expressed in the cells using the proteomics analysis described in the method section above.

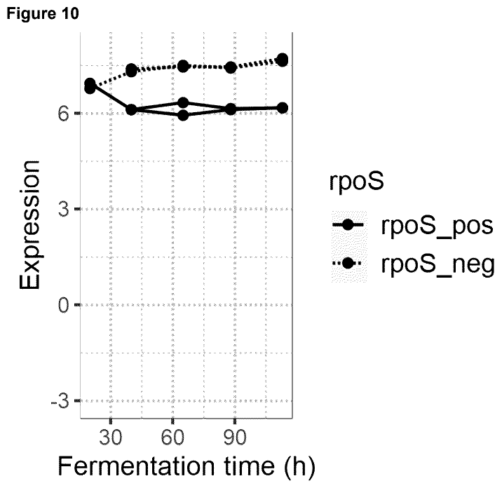

In example 7 it was shown that deletion of rpoS in the 2′FL producing E. coli strain increased the transcription of the heterologous gene futC. In the present example it can be seen that this increased transcription also resulted in increased protein level of the alpha-1,2-fucosyl-transferase, FutC, was increased in the strain where RpoS was deleted compared to the control strain (FIG. 10). Specifically, the increase is approximately 1.2 fold in log 2, which when reversed to log give 2.3 fold increase in FutC in the RpoS deleted strain.

This clearly indicates that reduction of RpoS levels in a cell expressing heterologous genes is not only useful in relation to HMO production, it can also be relevant for production of heterologous protein products using a genetically engineered host cell where the stress response sigma factor, such as RpoS or an equivalent, has been reduced or abolished.

### Example 9

Increased Endogenous Protein Expression in E. coli 2′FL Production Strain Lacking rpoS

In the present example it was investigated how the deletion of rpoS affected the expression of endogenous proteins that are relevant for HMO expression. Membrane bound proteins were excluded from the analysis since they may be difficult to access accurately using the proteomic analysis described in the methods above.

Specifically, in the fermentation described in example 6, where an E. coli 2′FL production strains with either an intact rpoS gene (Strain 1, rpoS_pos) or lacking rpoS (Strain 2, rpoS_neg) was fermented in a fed-batch fermentation, the samples collected at different timepoint after feed start (21, 41, 66, 90, 95, 113 hours) were analyzed for the amount of selected proteins expressed in the cells using the proteomics analysis described in the method section above. The proteins analyzed are listed in table 14.

TABLE 14
endogenous proteins analyzed using proteomics

| | | Relevance in |
|---|---|---|
| Protein | Function | HMO production |
| ManB | Pathway for GDP-fucose | 2′FL formation |
| ManC | production | |
| Gmd | | |
| Fcl | | |
| SucA | TCA cycle | Potential reason for reduced |
| SucB | | formation of glutamic acid |
| SucD | | |
| Protein | Function | Relevance in HMO production |
| gltA | | Potential reason for reduction |
| | | of acetate |

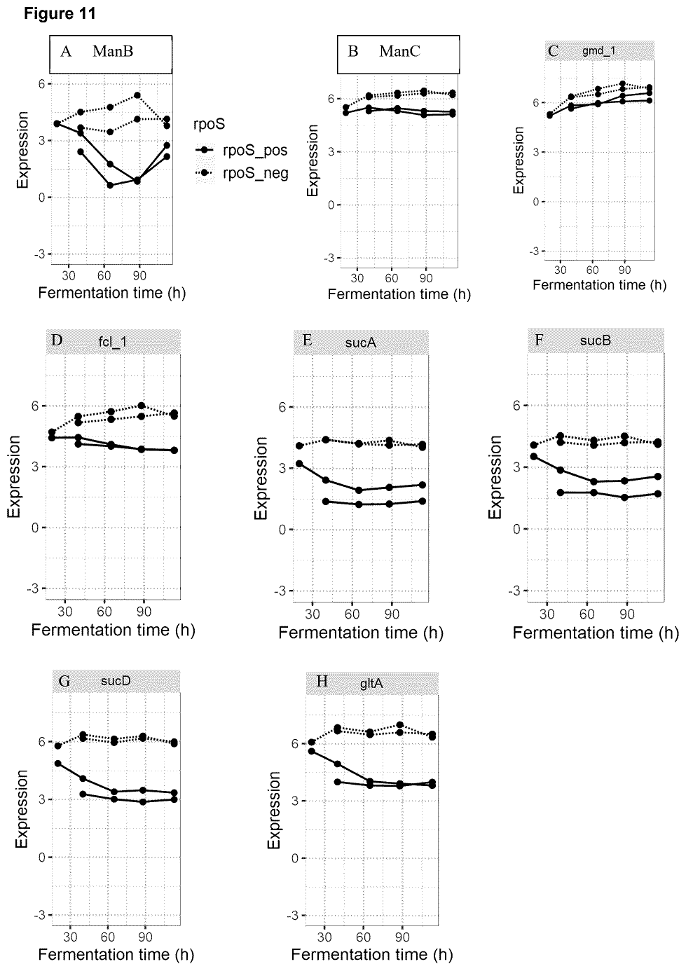

In FIG. 11 it can be seen that the levels of all the proteins in table 14 were increased when rpoS was deleted in the 2′FL producing E. coli strain as compared to the rpoS positive strain.

### Example 10

Additional Increase in Protein Expression in E. coli 2′FL Production Strain Having rpoS Deleted Combined with Overexpression of manA and manB

The formation of GDP-fucose is highly important in a cell producing fucosylated HMO's. In example 7 deletion of rpoS resulted in a significant increase in the transcription of manA and manB and in example 6 (FIG. 8) it was shown that deletion of rpoS in an E. coli 2′FL production strain increased the 2′FL yield in fermentation by 40%.

In the present example it was investigated if deletion of rpoS combined with overexpression of manA and manB could increase the 2′FL production even further. In the present example an E. coli 2′FL strain deleted of rpoS (Strain 2) and an E. coli 2′FL strain deleted of rpoS combined with overexpression of manA and manB (strain 14) were fermented in a fed-batch fermentation.

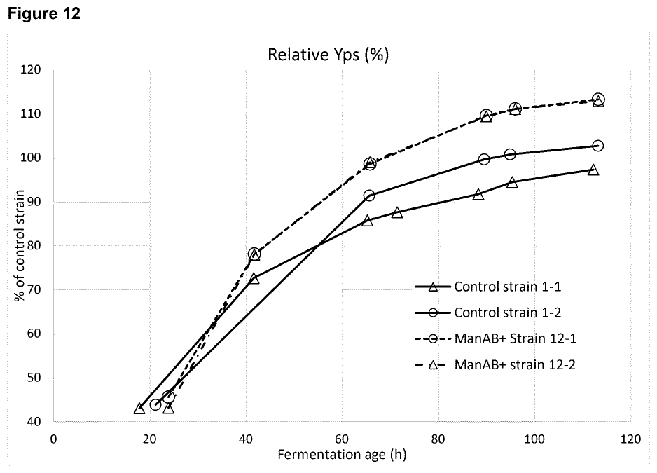

Samples were collected at different timepoints after feed start (21, 41, 66, 90, 95, 113 hours) and analyzed for the amount of 2′FL. FIG. 12 shows that deletion of rpoS combined with overexpression of manA and manB in an E. coli 2′FL production strain increased the 2′FL yield in fermentation by approximately 10% compared to a 2′FL production strain having rpoS deleted. The yield was measured as accumulated product yield per carbon source substrate

(Yps) (g 2′FL/g sucrose) and was normalized to the Yps of strain 2 (ΔrpoS) at the end of fermentation. Each strain was tested in fermentation in duplicates.

1. A genetically engineered cell comprising
a. at least one heterologous nucleic acid encoding a heterologous product or encoding a polypeptide required for the production of the heterologous product, and
a promoter, which is recognizable by a first sigma factor of the cell, regulating the transcription or expression of the heterologous nucleic acid, and
a second sigma factor with activity in the stationary-phase of the cell,
wherein the cell is genetically modified to reduce or abolish activity of the second sigma factor, and
wherein the genetically engineered cell is not a vaccine strain.

2. The genetically engineered cell according to claim 1, wherein the second sigma factor is selected from the group consisting of RpoS and SigB.

3. The genetically engineered cell according to claim 2, wherein the cell is genetically modified to reduce or abolish activity of the second sigma factor by
b. fully or partially inactivating a gene encoding RpoS or SigB, or
c. reducing or abolishing the function of one or more factors promoting RpoS or SigB function.

4. The genetically engineered cell according to claim 3, wherein the reducing or abolishing the function of one or more factors promoting RpoS function comprises fully or partially inactivating one or more genes selected from the group consisting of ArcZ, DksA, GadX, DsrA, DeaD, RprA and Crl, or a combination thereof.

5. The genetically engineered cell according to claim 2, wherein the cell is genetically modified to reduce or abolish activity of the second sigma factor by increasing the function of one or more factor(s) inhibiting RpoS or SigB function.

6. The genetically engineered cell according to claim 5, wherein the increasing the function of one or more factors inhibiting RpoS function comprises overexpressing one or more-nucleic acids selected from the group consisting of rssB, RNase III, H-NS, ArcA, CRP, Fur, MqsA, OxyS and CyaR, or a combination thereof.

7. (canceled)

8. The genetically engineered cell according claim 1, wherein the first sigma factor of the cell is selected from the group consisting of RpoD and SigA.

9. The genetically engineered cell according to claim 1, wherein the promoter comprises the consensus motif TT (G/C/T) (A/T) C (A/G) (n)₁₄₋₁₈ TA (T/A) (A/G) (A/T) T located at the 5′end region of the heterologous nucleic acid between 5 and 40 nucleotides upstream of the translation start codon AUG.

10. The genetically engineered cell according to claim 1, wherein the promoter is positively regulated by cAMP-bound CRP.

11. The genetically engineered cell according to claim 1, wherein the promoter is selected from the group consisting of SEQ ID NO: 35 (PglpF), 44 (Plac), 32 (PmglB_70UTR), 33 (PglpA_70UTR), 82 (PgatY) and 34 (PglpT_70UTR), or a functional homologue thereof with 95% identity to SEQ ID NO: 34, 44, 32, 33, 82, or 34.

12. The genetically engineered cell according to claim 1, wherein the heterologous nucleic acid is selected from the group consisting of one or more glycosyltransferase gene(s), one or more CMP-N-acetylneuraminic acid pathway gene(s), one or more transporter gene(s) and one or more nucleic acid(s) encoding the heterologous product to be produced by the genetically engineered cell.

13. The genetically engineered cell according to claim 1, wherein the heterologous product is one or more human milk oligosaccharide(s) (HMO(s)).

14. The genetically engineered cell according to claim 13, wherein the one or more HMOs are selected from the group consisting of lacto-N-triose II (LNT-II) lacto-N-tetraose (LNT), lacto-N-neotetraose (LNnT), lacto-N-neohexaose (LNnH), para-lacto-N-neohexaose (pLNnH), para-lacto-N-hexaose (pLNH), lacto-N-hexaose (LNH), 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II), fucosyl-lacto-N-neohexaose (FLNnH), 3′-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

15. The genetically engineered cell according to claim 13, wherein the genetically engineered cell comprises one or more heterologous nucleic acids encoding one or more heterologous glycosyltransferases selected from the group consisting of β-1,3-N-acetyl-glucosaminyltransferase(s), β-1,3-galactosyltransferase(s), β-1,4-galactosyltransferase(s), α-1,2-fucosyltransferase(s), α-1,3-fucosyltransferase, α-2,3-sialyltransferase(s) and α-2,6-sialyltransferase(s).

16. (canceled)

17. (canceled)

18. The genetically engineered cell according to claim 1, wherein the cell is a bacterium selected from the group consisting of Bacillus sp., Lactobacillus sp., Corynebacterium sp. Campylobacter sp., and E. coli.

19. The genetically engineered cell according to claim 18, wherein the E. coli is E. coli K-12.

20. A method for biosynthetically producing a heterologous product, comprising
a. providing a genetically engineered cell according to claim 1;
b. cultivating the genetically engineered cell in a culture medium under conditions permissive for the production of the heterologous product; and
c. recovering the heterologous product from the culture.

21. The method according to claim 20, wherein the pH during the cultivation is maintained above 6.0.

22. The method according to claim 20, wherein the cultivating comprises a fed-batch or continuous fed-batch fermentation where the carbon source is continuously feed to the fermentation broth keeping the fermentation under carbon limiting conditions.

23. The method according to claim 20, wherein the heterologous product is one or more human milk oligosaccharides (HMOs).

24. (canceled)

25. (canceled)

26. (canceled)
